(19) United States
(12) Patent Application Publication
(10) Pub. No.: US 2026/0216318 A1
(43) Pub. Date: Jul. 30, 2026

(54) GENE OF INTEREST RNA FORMULATIONS

(71) Applicant: Immorna (Hangzhou) Biotechnology Co., Ltd., Hangzhou (CN)

(72) Inventors: Zhijun Guo, Hangzhou (CN); Zihao Wang, Hangzhou (CN); Hongyue Wu, Hangzhou (CN)

(21) Appl. No.: 19/183,422

(22) Filed: Apr. 18, 2025

Related U.S. Application Data
(63) Continuation of application No. PCT/CN2023/125358, filed on Oct. 19, 2023.

(51) Int. Cl. A61K 39/215; A61K 39/00; A61P 37/04; C07K 14/005

(52) U.S. Cl. CPC A61K 39/215; A61P 37/04; C07K 14/005; A61K 2039/53; A61K 2039/55555; C12N 2770/20022; C12N 2770/20034

(57) ABSTRACT

The disclosure relates to RNA molecules with multivalent capability against multiple viral strains or antigens and methods of making the same.

### CROSS-REFERENCE

This application is a continuation of International Application Number PCT/CN2023/125358, which claims the benefit of and priority to PCT/CN2022/126258, filed on Oct. 19, 2022, which is hereby incorporated herein by reference in their entirety for all purposes.

### SEQUENCE LISTING

The instant application contains a Sequence Listing which has been submitted electronically in XML format and is hereby incorporated by reference in its entirety. Said XML copy, created on Oct. 11, 2023, is named IMO-004WO_SL.xml and is 67,507 bytes in size.

### BACKGROUND

RNA is a single-stranded polynucleotide molecule that can encode the expression of a specific protein, such as a disease-specific antigen, upon reaching the cytoplasm. The successful synthesis of RNA by in vitro transcription (IVT) initiated research on its use as a therapeutic agent. Currently, RNA vaccines against COVID-19 are widely used and have been successful against SARS-CoV-2. However, the current vaccines generally only target one variant.

New variants of viruses (e.g., SARS-CoV-2) continue to appear and vaccines are needed that can target these new variants as well as a combination of variants. To address this, multivalent vaccines are needed to deal with new mutant strains of human infection. Provided herein, in certain embodiments, are improved RNA vaccines with multivalent capability and safety against multiple viral variants.

### BRIEF SUMMARY

Described herein are RNA vaccines with multivalent capability against multiple variants of viruses, such as SARS-CoV-2 virus. Such RNA vaccines are designed to effectively integrate different sequences of genes that express the protein of different viral strains into one platform, creating a multivalent RNA vaccine. Moreover, the presence of an oligomerization domain, such as the T4 Foldon domain, facilitates stability to express multiple antigens and subsequently enhances immune potency. Further, RNA vaccines described herein avoid complicated mixing processes, over-dosing, burden of excipients, and toxicity safety concerns compared to traditional vaccines. Further provided herein are methods for preparing the RNA vaccines, for example, by providing the RNA with a LNP composition.

In one aspect, provided herein is purified ribonucleic acid (RNA) molecule comprising: a) a first nucleotide sequence encoding a first antigen, or an antigenic fragment thereof, from a first virus variant; b) a second nucleotide sequence encoding a multimerization domain; and c) a third nucleotide sequence encoding a second antigen, or an antigenic fragment thereof, from a second virus variant, wherein the first virus variant and the second virus variant are different, and wherein the first nucleotide sequence, the second nucleotide sequence, and the third nucleotide sequence are operably linked to each other in a 5′-to-3′ direction.

In some embodiments, the RNA is a messenger RNA (mRNA). In some embodiments, the RNA is a self-replicating RNA. In some embodiments, the RNA is not a self-replicating RNA.

In some embodiments, the virus is selected from the group consisting of a Influenza virus, Rabies virus, respiratory syncytial virus (RSV), and coronavirus.

In some embodiments, wherein the virus is selected from the group consisting of 229E (alpha coronavirus), NL63 (alpha coronavirus), OC43 (beta coronavirus), HKU1 (beta coronavirus), MERS-CoV (MERS), SARS-CoV (SARS), and SARS-CoV-2 (COVID-19).

In some embodiments, the virus is SARS-CoV-2.

In some embodiments, the first virus variant or the second virus variant is selected from the group consisting of Wuhan-Hu-1, Alpha, Beta, Gamma, Delta, Epsilon Eta, Iota, Kappa, 1.617.3, Mu, Zeta, and Omicron.

In some embodiments, the first virus variant or the second virus variant is selected from the group consisting of Wuhan-Hu-1 and Delta.

In some embodiments, the first antigen, or the antigenic fragment thereof, or the second antigen, or the antigenic fragment thereof, comprises a viral envelope protein, viral spike protein, viral membrane protein, or viral capsid protein, the first antigen, or the antigenic fragment thereof, or the second antigen, or the antigenic fragment thereof, comprises a viral spike protein.

In some embodiments, the first antigen, or the antigenic fragment thereof, or the second antigen, or the antigenic fragment thereof, comprises a receptor-binding domain (RBD).

In some embodiments, the RBD domain is a RBD domain from Wuhan-Hu-1 variant or Delta variant.

In some embodiments, the first antigen, or the antigenic fragment thereof, comprises the RBD domain from the Delta variant and the second antigen, or the antigenic fragment thereof, comprises the RBD domain of the Wuhan-Hu-1 variant.

In some embodiments, the virus is an influenza A virus.

In some embodiments, the influenza A virus is selected from the group consisting of H1N1 (PR8), H2N2, H1N2, H3N2 (Udorn), N2-NA, Wyo03, PC73, H5N1. H5N2, H5N8, H5N9, H7N2, H7N3, H7N7, H9N2, H10N7, and H10N3.

In some embodiments, the first virus variant or the second virus variant is selected from the group consisting of H1N1 (PR8) and H3N2 (Udorn).

In some embodiments, the first antigen, or the antigenic fragment thereof, or the second antigen, or the antigenic fragment thereof, comprises an RNA polymerase subunit, hemagglutinin (HA), nucleoprotein (NP), neuraminidase (NA), matrix protein 1 (M1), matrix protein 2 (M2), non-structural protein NS1, or non-structural protein NEP.

In some embodiments, the first antigen, or the antigenic fragment thereof, or the second antigen, or the antigenic fragment thereof, comprises a hemagglutinin (HA) protein.

In some embodiments, the HA protein is from H1N1 (PR8) or H3N2 (Udorn).

In some embodiments, wherein the first antigen, or the antigenic fragment thereof, comprises an HA protein from H1N1 (PR8) and the second antigen, or the antigenic fragment thereof, comprises an HA protein from H3N2 (Udorn).

In some embodiments, the multimerization domain is selected from the group consisting of a dimerization domain, trimerization domain, and a tetramerization domain.

In some embodiments, wherein the multimerization domain is selected from the group consisting of enterobacteria phage T4, GCN4pII, GCN4-pLI, and p53.

In some embodiments, the multimerization domain comprises a leucine zipper or a fibritin foldon domain.

In some embodiments, the multimerization domain comprises a trimerization domain.

In some embodiments, the fibritin foldon domain is the trimerization domain from enterobacteria phage T4.

In some embodiments, the trimerization domain is encoded by a nucleotide sequence having at least 90% sequence identity to the sequence of SEQ ID NO.: 1.

In some embodiments, the RNA molecules described herein further comprise a sequence encoding a first linker connecting the first antigen, or the antigenic fragment thereof, to the multimerization domain.

In some embodiments, the RNA molecules described herein further comprise a sequence encoding a second linker connecting the second antigen, or the antigenic fragment thereof, to the multimerization domain.

In some embodiments, the first linker encodes an amino acid sequence comprising at least 5 to about 50 amino acids.

In some embodiments, the second linker encodes an amino acid sequence comprising s at least 5 to about 50 amino acids.

In some embodiments, the first linker encodes the amino acid sequence selected from the group consisting of (GS)n (SEQ ID NO: 9), (G2S)n (SEQ ID NO: 10), (G3S)n (SEQ ID NO: 11), (G4S)n (SEQ ID NO: 32), and (G)n (SEQ ID NO: 33), and wherein n is an integer from 2 to 20. In some embodiments, the second linker encodes the amino acid sequence selected from the group consisting of (GS)n (SEQ ID NO: 9), (G2S)n (SEQ ID NO: 10), (G3S)n (SEQ ID NO: 11), (G4S)n (SEQ ID NO: 32), and (G)n (SEQ ID NO: 33), and wherein n is an integer from 2 to 20.

In some embodiments, the first linker encodes the amino acid sequence selected from the group consisting of (GGSGGD)n (SEQ ID NO: 34) or (GGSGGE)n (SEQ ID NO: 35), and wherein n is an integer from 2 to 6.

In some embodiments, the second linker encodes the amino acid sequence selected from the group consisting of (GGSGGD)n (SEQ ID NO: 34) or (GGSGGE)n (SEQ ID NO: 35), and wherein n is an integer from 2 to 6.

In some embodiments, the first linker encodes the amino acid sequence selected from the group consisting of (GGGSGSGGGGS)n (SEQ ID NO: 36) and (GGGGGPGGGGP) n (SEQ ID NO: 37), and wherein n is an integer from 1 to 3. In some embodiments, the second linker encodes the amino acid sequence selected from the group consisting of (GGGSGSGGGGS)n (SEQ ID NO: 36) and (GGGGGPGGGGP)n (SEQ ID NO: 37), and wherein n is an integer from 1 to 3.

In some embodiments, the first linker encodes the amino acid sequence selected from the group consisting of (GX)n, (GGX)n, (GGGX)n, (GGGGX)n, and (GzX)n, wherein z is between 1 and 20, and wherein n is at least 8.

In some embodiments, the second linker encodes the amino acid sequence selected from the group consisting of (GX)n, (GGX)n, (GGGX)n, (GGGGX)n, and (GzX)n, wherein z is between 1 and 20, and wherein n is at least 8.

In some embodiments, X is serine, aspartic acid, glutamic acid, threonine, or proline.

In some embodiments, first linker encodes GGSG (SEQ ID NO: 38). In some embodiments, second linker encodes GGSG (SEQ ID NO: 38).

In some embodiments, the first linker encodes GGSLGGGGSGS (SEQ ID NO: 39).

In some embodiments, the second linker encodes GGSLGGGGSGS (SEQ ID NO: 39).

In some embodiments, the first linker is encoded by the nucleotide sequence according to SEQ ID NO: 5 or SEQ ID NO: 6.

In some embodiments, the second linker is encoded by the nucleotide sequence according to SEQ ID NO: 5 or SEQ ID NO: 6.

In some embodiments, the first antigen, or the antigenic fragment thereof, is encoded by a nucleotide sequence having at least 90% sequence identity to the sequence of any one of SEQ ID NOs.: 3-4, 7, and 8.

In some embodiments, the second antigen, or the antigenic fragment thereof, is encoded by a nucleotide sequence having at least 90% sequence identity to the sequence of any one of SEQ ID NOs.: 3-4, 7, and 8.

In some embodiments, the first antigen, or the antigenic fragment thereof, comprises a sequence having at least 90% sequence identity to the sequence of any one of SEQ ID NOs: 19, 20, 22, and 23.

In some embodiments, the second antigen, or the antigenic fragment thereof, comprises a sequence having at least 90% sequence identity to the sequence of any one of SEQ ID NOs: 19, 20, 22, and 23.

In some embodiments, the purified RNA further comprises a m7GpppNm-, where Nm denotes any nucleotide with a 2′ O methylation (Cap 1) 5′ to the first nucleotide sequence.

In some embodiments, the purified RNA further comprises a 5′ UTR 3′ to the Cap 1 and 5′ to the first nucleotide sequence.

In some embodiments, the purified RNA further comprises a sequence encoding a signal peptide 3′ to the 5′ UTR and 5′ to the first nucleotide sequence.

In some embodiments, the purified RNA further comprises a sequence encoding a 3′ UTR 3′ to the second nucleotide sequence.

In one aspect, provided herein, is a purified RNA molecule comprising a sequence having at least 90% sequence identity to the sequence of any one of SEQ ID NOs: 26-31.

In one aspect, provided herein, is a purified RNA molecule comprising a sequence having at least 90% sequence identity to the sequence of SEQ ID NO: 26.

In one aspect, provided herein, is a purified RNA molecule comprising a sequence having at least 90% sequence identity to the sequence of SEQ ID NO: 27.

In one aspect, provided herein, is a purified RNA molecule comprising a sequence having at least 90% sequence identity to the sequence of SEQ ID NO: 28.

In one aspect, provided herein, is a purified RNA molecule comprising a sequence having at least 90% sequence identity to the sequence of SEQ ID NO: 29.

In one aspect, provided herein, is a purified RNA molecule comprising a sequence having at least 90% sequence identity to the sequence of SEQ ID NO: 30.

In one aspect, provided herein, is a purified RNA molecule comprising a sequence having at least 90% sequence identity to the sequence of SEQ ID NO: 31.

In one aspect, provided herein, is a purified RNA molecule comprising, from 5′ to 3′, a) a m7GpppNm-, where Nm denotes any nucleotide with a 2′ O methylation (Cap 1); b) a 5′ UTR; c) a first nucleotide sequence encoding a signal peptide; d) a second nucleotide sequence encoding a first antigen, or an antigenic fragment thereof, from a wild-type SARS-CoV-2 virus or a first virus variant selected from the group consisting of: a Wuhan-Hu-1 variant, an Omicron variant, and a Delta variant; e) a third nucleotide sequence encoding a first linker; f) a fourth nucleotide sequence encoding a T4 foldon domain; g) a fifth nucleotide sequence encoding a second linker; h) a sixth nucleotide sequence encoding a second antigen, or an antigenic fragment thereof, from a wild-type SARS-CoV-2 virus or a second virus variant selected from the group consisting of: a Wuhan-Hu-1 variant, an Omicron variant, and a Delta variant; i) a 3′ UTR; and j) a poly A tail.

In one aspect, provided herein, is a purified RNA molecule comprising, from 5′ to 3′, a) a m7GpppNm-, where Nm denotes any nucleotide with a 2′ O methylation (Cap 1); b) a 5′ UTR; c) a first nucleotide sequence encoding a signal peptide; d) a second nucleotide sequence encoding a first antigen, or an antigenic fragment thereof, from a wild-type SARS-CoV-2 virus or a first virus variant selected from an Omicron variant; e) a third nucleotide sequence encoding a first linker; f) a fourth nucleotide sequence encoding a T4 foldon domain; g) a fifth nucleotide sequence encoding a second linker; h) a sixth nucleotide sequence encoding a second antigen, or an antigenic fragment thereof, from a wild-type SARS-CoV-2 virus; i) a 3′ UTR; and j) a poly A tail.

In one aspect, provided herein, is a purified RNA molecule comprising, from 5′ to 3′, a) a m7GpppNm-, where Nm denotes any nucleotide with a 2′ O methylation (Cap 1); b) a 5′ UTR; c) a first nucleotide sequence encoding a signal peptide; d) a second nucleotide sequence encoding a first antigen, or an antigenic fragment thereof, from a wild-type SARS-CoV-2 virus; e) a third nucleotide sequence encoding a first linker; f) a fourth nucleotide sequence encoding a T4 foldon domain; g) a fifth nucleotide sequence encoding a second linker; h) a sixth nucleotide sequence encoding a second antigen, or an antigenic fragment thereof, from an Omicron variant; i) a 3′ UTR; and j) a poly A tail.

In one aspect, provided herein, is a purified RNA molecule comprising, from 5′ to 3′, a) a m7GpppNm-, where Nm denotes any nucleotide with a 2′ O methylation (Cap 1); b) a 5′ UTR; c) a first nucleotide sequence encoding a first antigen, or an antigenic fragment thereof, from an influenza virus selected from the group consisting of H1N1 (PR8), H2N2, H1N2, H3N2 (Udorn), N2-NA, Wyo03, PC73, H5N1. H5N2, H5N8, H5N9, H7N2, H7N3, H7N7, H9N2, H10N7, and H10N3; d) optionally, a second nucleotide sequence encoding a first linker; e) optionally, a third nucleotide sequence encoding a T4 foldon domain; f) optionally, a fourth nucleotide sequence encoding a second linker; g) optionally, a fifth nucleotide sequence encoding a second antigen, or an antigenic fragment thereof, from an influenza virus selected from the group consisting of H1N1 (PR8), H2N2, H1N2, H3N2 (Udorn), N2-NA, Wyo03, PC73, H5N1. H5N2, H5N8, H5N9, H7N2, H7N3, H7N7, H9N2, H10N7, and H10N3; h) a 3′ UTR; and i) a poly A tail.

In one aspect, provided herein, is a purified RNA molecule comprising, from 5′ to 3′, a) a m7GpppNm-, where Nm denotes any nucleotide with a 2′ O methylation (Cap 1); b) a 5′ UTR; c) a first nucleotide sequence encoding a first antigen, or an antigenic fragment thereof, from a H1N1 (PR8) influenza virus; d) a second nucleotide sequence encoding a first linker; e) a third nucleotide sequence encoding a T4 foldon domain; f) a fourth nucleotide sequence encoding a second linker; g) a fifth nucleotide sequence encoding a second antigen, or an antigenic fragment thereof, from an influenza virus selected from a H3N2 (Udorn) influenza virus; h) a 3′ UTR; and i) a poly A tail.

In one aspect, provided herein, is a purified RNA molecule comprising, from 5′ to 3′, a) a m7GpppNm-, where Nm denotes any nucleotide with a 2′ O methylation (Cap 1); b) a 5′ UTR; c) a nucleotide sequence encoding a first antigen, or an antigenic fragment thereof, from a H1N1 (PR8) influenza virus; d) a 3′ UTR; and e) a poly A tail.

In one aspect, provided herein, is a purified RNA molecule comprising, from 5′ to 3′, a) a m7GpppNm-, where Nm denotes any nucleotide with a 2′ O methylation (Cap 1); b) a 5′ UTR; d) a nucleotide sequence encoding a first antigen, or an antigenic fragment thereof, from a H3N2 (Udorn) influenza virus; d) a 3′ UTR; and e) a poly A tail.

In one aspect, provided herein, is an isolated polypeptide encoded by the purified RNA molecule of any of the preceding claims.

In one aspect, provided herein, is composition comprising any of the purified RNA molecules described herein and a delivery vehicle.

In some embodiments, the delivery vehicle comprises a lipid nanoparticle (LNP).

In some embodiments, the LNP comprises an ionizable lipid.

In some embodiments, the LNP comprises an ionizable lipid:cholesterol:DSPC:DMG-PEG2000 ratio of about 48:40:10:2, and a LNP:RNA (N:P) ratio of about 8:1.

In some embodiments, the composition comprises a particle size of no more than about 300 nanometers (nm).

In some embodiments, the composition comprises a particle size of about 50 to about to 300 nm. In some embodiments, the composition comprises a particle size of no more than about 150 nm. In some embodiments, the composition comprises a particle size no more than about 140 nm. In some embodiments, the composition comprises a particle size of about 50 nm to about 140 nm. In some embodiments, the composition comprises a particle size of about 125 nm.

In some embodiments, the RNA is lyophilized.

In some embodiments, the RNA is adsorbed to the surface of the LNP.

In some embodiments, the RNA is encapsulated by the LNP.

In some embodiments, the RNA encapsulated by the LNP is lyophilized.

In one aspect, provided herein, is a kit comprising any of the purified RNA molecules described herein, a delivery vehicle, and instructions for use.

In one aspect, provided herein, is a method of treating or preventing disease in a subject comprising administering to the subject an effective amount any of the compositions described herein.

In some embodiments, the disease is caused by a virus is selected from the group consisting of a Influenza virus, Rabies virus, respiratory syncytial virus (RSV) and coronavirus.

In some embodiments, the disease is caused by coronavirus. In some embodiments, the coronavirus is selected from the group consisting of 229E (alpha coronavirus), NL63 (alpha coronavirus), OC43 (beta coronavirus), HKU1 (beta coronavirus), MERS-CoV (MERS), SARS-CoV (SARS), and SARS-CoV-2 (COVID-19). In some embodiments, the coronavirus is SARS-CoV-2.

In some embodiments, the RNA adsorbed to the surface of the LNP is in a liquid state, and optionally at a temperature of 2-8° C.

In some embodiments, the RNA and LNP is administered to the subject intramuscularly.

In some embodiments, the lyophilized RNA encapsulated in the LNP is mixed with sterile water.

In some embodiments, the lyophilized RNA encapsulated in the LNP and sterile water are administered to the subject intramuscularly.

In some embodiments, the RNA adsorbed to the surface of the LNP in a liquid state or lyophilized RNA encapsulated in the LNP is administered to the subject at least two times.

In some embodiments, the LNP in a liquid state or lyophilized RNA encapsulated in the LNP is administered once every one, two, three, or four weeks.

In one aspect, provided herein, is a method for stimulating an immune response in a subject comprising administering to the subject an effective amount of any of the compositions described herein.

In one aspect, provided herein, is a method for preparing a RNA-LNP composition comprising: mixing an ethanol phase comprising one or more lipids and an aqueous phase comprising the any of the purified RNA molecules described herein, and purifying the RNA-LNP generated from step a).

In some embodiments, the one or more lipids comprises an ionizable lipid. In some embodiments, the one or more lipids comprises cholesterol. In some embodiments, the one or more lipids comprises a phospholipid. In some embodiments, the one or more lipids is pegylated.

In some embodiments, the N:P ratio is about 6.5 to about 9.

In some embodiments, the aqueous phase consists of tris, sodium chloride, and sucrose.

In one aspect, provided herein, is a kit comprising any of the compositions described herein, a delivery vehicle, and instructions for use.

### BRIEF DESCRIPTION OF THE DRAWINGS

FIG. 1A shows an exemplary schema of a protein encoded by an RNA structure described herein designed to simultaneously express different receptor binding domain (RBD) proteins from various coronavirus strains through a linker and T4 Foldon oligomerization domain. FIG. 1A discloses SEQ ID NOS 38-39, respectively, in order of appearance.

FIG. 1B shows a schematic drawing of an RNA structure with two antigen sequences with an oligomerization domain in between the two antigen sequences.

FIG. 2 shows a Western blot that was probed for the RBD protein of SARS-CoV-2 from a sample of BHK-21 cells transfected with 2 microgram (μg) of the lipid nanoparticle (LNP)-encapsulated RNA vaccine described herein.

FIG. 3 shows a treatment protocol of BALB/c mice vaccinated with a RNA-LNP vaccine described herein, which encodes the RBD protein of SARS-CoV-2.

FIG. 4 shows a bar graph of the geometric mean titer (GMT) of BALB/c mice vaccinated with the RNA-LNP vaccine described herein, which encodes the RBD protein of SARS-CoV-2 at the indicated days.

FIG. 5 shows a bar graph of the SARS-CoV-2 neutralizing antibody for cross-protection with different SARS-CoV-2 strains.

FIG. 6 shows an exemplary schematic of a mRNA described herein.

FIG. 7 shows a schematic illustration of immunization of mice.

FIGS. 8A-8B show bars graphs of antigen-specific binding antibody against SARS-CoV-2 RBD polypeptides of the SARS-CoV-2 ancestral strain (FIG. 8A) and that of the Omicron BA. 1 strain (FIG. 8B) as measured by ELISA at different time-point. *, below starting lowest dilution fold; **, above the highest dilution folds.

FIG. 9 shows a graph showing mouse serum neutralizing antibody titers against SARS-CoV-2 pseudoviruses (ancestral, Beta, Delta, Omicron BA.1, BA.2.12.1, and BA.4/.5 strains).

FIG. 10 shows a graph showing percentages of IFN-γ secreting splenocytes isolated from mice immunized the mRNA-LNP vaccine by ICS upon stimulation with antigen peptide pools. ***, p<0.001 compared with control group.

FIG. 11 shows a group of schematic drawing of RNA structures with two antigen sequences with an oligomerization domain in between the two antigen sequences.

FIG. 12 shows a bar graph of the geometric mean titer (GMT) of BALB/c mice vaccinated with the RNA-LNP vaccine having Structure 1, Structure 2, or Structure 3, which encode a RBD-WT and RBD-BA.1 antigen protein of SARS-CoV-2. The serum samples were collected on day 28. Each bar shown is a geometric mean (N=4) of IgG titers of individual mouse sera in each group.

FIG. 13 shows a bar graph of the hemagglutination inhibition (HAI) of BALB/c mice vaccinated with the RNA-LNP vaccine having Structure 4, Structure 5, or Structure 6, which encode influenza antigens as indicated. The serum samples were collected on day 28. Each bar shown is a geometric mean (N=5) of HAI titers of individual mouse sera in each group.

### DETAILED DESCRIPTION

Traditional vaccines generally employ one antigen sequence to provide immunity towards a particular virus. Variants of viruses can occur, and depending on the variant, may cause the immunity gained from a traditional vaccine to be moot. Therefore, it is advantageous to create a vaccine that can provide immunity of more than one virus variant. The RNA molecules described herein comprise multiple antigen sequences to provide immunity to multiple variants of virus.

### Nucleic Acids of the Disclosure

Provided herein, in some embodiments, are purified ribonucleic acid (RNA) molecules comprising: a) a first nucleotide sequence encoding a first antigen, or an antigenic fragment thereof, from a first virus variant; b) a second nucleotide sequence encoding a multimerization domain; and c) a third nucleotide sequence encoding a second antigen, or an antigenic fragment thereof, from a second virus variant, wherein the first virus variant and the second virus variant are different, and wherein the first nucleotide sequence, the second nucleotide sequence, and the third nucleotide sequence are operably linked to each other in a 5′-to-3′ direction.

The RNA molecules described herein, in some embodiments, are messenger RNA (mRNA). “Messenger RNA” (mRNA) refers to one type RNA molecule that encodes a (at least one) polypeptide (a naturally-occurring, non-naturally-occurring, or modified polymer of amino acids) and can be translated to produce the encoded polypeptide in vitro, in vivo, in situ or ex vivo. The skilled artisan will appreciate that, except where otherwise noted, RNA molecule sequences will recite “T”s in a representative DNA sequence, but where the sequence represents RNA (e.g., mRNA), the “T”s would be substituted by “U”s.

Thus, any of the RNA molecules encoded by a DNA identified by a particular sequence identification number may also comprise the corresponding RNA (e.g., mRNA or other coding RNA) sequence encoded by the DNA, where each “T” of the DNA sequence is substituted with “U.”

It should be understood that the RNA molecules as provided herein are synthetic molecules, i.e., they are not naturally-occurring molecules. That is, the RNA molecules of the present disclosure are isolated (i.e., purified) RNA molecules. As is known in the art, “isolated RNA molecules or polynucleotides” refer to polynucleotides that are substantially physically separated from other cellular material (e.g., separated from cells and/or systems that produce the polynucleotides) or from other material that hinders their use in the vaccines or therapeutics of the present disclosure. Isolated RNA molecules are substantially pure in that they have been substantially separated from the substances with which they may be associated in living or viral systems. Thus, RNA molecules are not associated with living or viral systems, such as cells or viruses. The RNA molecules do not include viral components (e.g., viral capsids, viral enzymes, or other viral proteins, for example, those needed for viral-based replication), and the RNA molecules are not packaged within, encapsulated within, linked to, or otherwise associated with a virus or viral particle.

Any sequence may be codon optimized. Codon optimization methods are known in the art. Codon optimization, in some embodiments, is used to match codon frequencies in target and host organisms to ensure proper folding; bias GC content to increase RNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g., glycosylation sites); add, remove or shuffle protein domains; insert or delete restriction sites; modify ribosome binding sites and RNA degradation sites; adjust translational rates to allow the various domains of the protein to fold properly; or reduce or eliminate problem secondary structures within the polynucleotide. In some embodiments, the open reading frame (ORF) sequence is optimized using optimization algorithms. In some embodiments, a sequence encoding an antigen, e.g., a spike protein of SARS-CoV-2, is codon optimized.

In some embodiments, the RNA includes at least one RNA molecule encoding at least one antigenic polypeptide having at least one of: a modification, at least one 5′ terminal cap, and formulation with a lipid nanoparticle. 5′-capping of polynucleotides may be completed concomitantly during the in vitro-transcription reaction using the following chemical RNA cap analogs to generate the 5′-guanosine cap structure according to manufacturer protocols: 3′-O-Me-m7G(5′)ppp(5′) G [the ARCA cap]; G(5′)ppp(5′)A; G(5′)ppp(5′)G; m7G(5′)ppp(5′)A; m7G(5′)ppp(5′)G. 5′-capping of modified RNA may be completed post-transcriptionally using a Vaccinia Virus Capping Enzyme to generate the “Cap 0” structure: m7G(5′)ppp(5′)G. Cap 1 structure may be generated using both Vaccinia Virus Capping Enzyme and a 2′-O methyl-transferase to generate: m7G(5′)ppp(5′)G-2′-O-methyl. Cap 2 structure may be generated from the Cap 1 structure followed by the 2′-O-methylation of the 5′-antepenultimate nucleotide using a 2′-O methyl-transferase. Cap 3 structure may be generated from the Cap 2 structure followed by the 2′-O-methylation of the 5′-preantepenultimate nucleotide using a 2′-O methyl-transferase. Enzymes may be derived from a recombinant source.

In some embodiments, an RNA molecule described herein comprises a Cap (e.g., m7G (Cap 0), m7GpppNm-, where Nm denotes any nucleotide with a 2′ O methylation (Cap 1), N6,2′-O-dimethyladenosine (m6AM), m7G(5′)ppp(5′)G (mCAP), or anti-reverse cap analogs (ARCA), optionally m7G or m7GpppNm—where Nm denotes any nucleotide with a 2′ O methylation). In some embodiments, an RNA molecule described herein comprises a m7G (Cap 0) cap. In some embodiments, an RNA molecule described herein comprises a m7GpppNm-, where Nm denotes any nucleotide with a 2′ O methylation (Cap 1) cap. In some embodiments, an RNA molecule described herein comprises a N6,2′-O-dimethyladenosine (m6AM) cap. In some embodiments, an RNA molecule described herein comprises a m7G(5′)ppp(5′)G (mCAP) cap. In some embodiments, an RNA molecule described herein comprises anti-reverse cap analogs (ARCA).

In some embodiments, an RNA molecule described herein comprises a 5′ UTR. In some embodiments, the 5′ UTR is 5′ of the first nucleotide sequence encoding the first antigen, or the antigenic fragment thereof, from the first virus variant. In some embodiments, an RNA molecule described herein comprises a 3′ UTR. In some embodiments, the 3′ UTR is 3′ of the second nucleotide sequence encoding the second antigen, or the antigenic fragment thereof, from the second virus variant.

In some embodiments, the 5′ UTR comprises a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 24. In some embodiments, the 5′ UTR comprises a sequence having at least about 80% identity to SEQ ID NO: 24. In some embodiments, the 5′ UTR comprises a sequence having at least about 85% identity to SEQ ID NO: 24. In some embodiments, the 5′ UTR comprises a sequence having at least about 90% identity to SEQ ID NO: 24. In some embodiments, the 5′ UTR comprises a sequence having at least about 95% identity to SEQ ID NO: 24. In some embodiments, the 5′ UTR comprises a sequence having at least about 96% identity to SEQ ID NO: 24. In some embodiments, the 5′ UTR comprises a sequence having at least about 97% identity to SEQ ID NO: 24. In some embodiments, the 5′ UTR comprises a sequence having at least about 98% identity to SEQ ID NO: 24. In some embodiments, the 5′ UTR comprises a sequence having at least about 99% identity to SEQ ID NO: 24. In some embodiments, the 5′ UTR comprises a sequence having 100% identity to SEQ ID NO: 24.

In some embodiments, the 5′ UTR is encoded by a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 14. In some embodiments, the 5′ UTR is encoded by a sequence having at least about 80% identity to SEQ ID NO: 14. In some embodiments, the 5′ UTR is encoded by a sequence having at least about 85% identity to SEQ ID NO: 14. In some embodiments, the 5′ UTR is encoded by a sequence having at least about 90% identity to SEQ ID NO: 14. In some embodiments, the 5′ UTR is encoded by a sequence having at least about 95% identity to SEQ ID NO: 14. In some embodiments, the 5′ UTR is encoded by a sequence having at least about 96% identity to SEQ ID NO: 14. In some embodiments, the 5′ UTR is encoded by a sequence having at least about 97% identity to SEQ ID NO: 14. In some embodiments, the 5′ UTR is encoded by a sequence having at least about 98% identity to SEQ ID NO: 14. In some embodiments, the 5′ UTR is encoded by a sequence having at least about 99% identity to SEQ ID NO: 14. In some embodiments, the 5′ UTR is encoded by a sequence having 100% identity to SEQ ID NO: 14.

In some embodiments, the 3′ UTR comprises a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 120% sequence identity to SEQ ID NO: 25. In some embodiments, the 3′ UTR comprises a sequence having at least about 80% identity to SEQ ID NO: 25. In some embodiments, the 3′ UTR comprises a sequence having at least about 85% identity to SEQ ID NO: 25. In some embodiments, the 3′ UTR comprises a sequence having at least about 90% identity to SEQ ID NO: 25. In some embodiments, the 3′ UTR comprises a sequence having at least about 95% identity to SEQ ID NO: 25. In some embodiments, the 3′ UTR comprises a sequence having at least about 96% identity to SEQ ID NO: 25. In some embodiments, the 3′ UTR comprises a sequence having at least about 97% identity to SEQ ID NO: 25. In some embodiments, the 3′ UTR comprises a sequence having at least about 98% identity to SEQ ID NO: 25. In some embodiments, the 3′ UTR comprises a sequence having at least about 99% identity to SEQ ID NO: 25. In some embodiments, the 3′ UTR comprises a sequence having 100% identity to SEQ ID NO: 25.

In some embodiments, the 3′ UTR is encoded by a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 120% sequence identity to SEQ ID NO: 15. In some embodiments, the 3′ UTR is encoded by a sequence having at least about 80% identity to SEQ ID NO: 15. In some embodiments, the 3′ UTR is encoded by a sequence having at least about 85% identity to SEQ ID NO: 15. In some embodiments, the 3′ UTR is encoded by a sequence having at least about 90% identity to SEQ ID NO: 15. In some embodiments, the 3′ UTR is encoded by a sequence having at least about 95% identity to SEQ ID NO: 15. In some embodiments, the 3′ UTR is encoded by a sequence having at least about 96% identity to SEQ ID NO: 15. In some embodiments, the 3′ UTR is encoded by a sequence having at least about 97% identity to SEQ ID NO: 15. In some embodiments, the 3′ UTR is encoded by a sequence having at least about 98% identity to SEQ ID NO: 15. In some embodiments, the 3′ UTR is encoded by a sequence having at least about 99% identity to SEQ ID NO: 15. In some embodiments, the 3′ UTR is encoded by a sequence having 100% identity to SEQ ID NO: 15.

In some embodiments, an RNA molecule described herein comprises a nucleotide sequence encoding a signal peptide. In some embodiments, the signal peptide is encoded by a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 2. In some embodiments, the signal peptide is encoded by a sequence having at least about 80% identity to SEQ ID NO: 2. In some embodiments, the signal peptide is encoded by a sequence having at least about 85% identity to SEQ ID NO: 2. In some embodiments, the signal peptide is encoded by a sequence having at least about 90% identity to SEQ ID NO: 2. In some embodiments, the signal peptide is encoded by a sequence having at least about 95% identity to SEQ ID NO: 2. In some embodiments, the signal peptide is encoded by a sequence having at least about 96% identity to SEQ ID NO: 2. In some embodiments, the signal peptide is encoded by a sequence having at least about 97% identity to SEQ ID NO: 2. In some embodiments, the signal peptide is encoded by a sequence having at least about 98% identity to SEQ ID NO: 2. In some embodiments, the signal peptide is encoded by a sequence having at least about 99% identity to SEQ ID NO: 2. In some embodiments, the signal peptide is encoded by a sequence having 100% identity to SEQ ID NO: 2.

In some embodiments, an RNA molecule described herein comprises a polyA tail (i.e., SEQ ID NO: 16).

Provided herein, in certain embodiments, are purified RNA molecules comprising, from 5′ to 3′: a) a m7GpppNm-, where Nm denotes any nucleotide with a 2′ O methylation (Cap 1); b) a 5′ UTR; c) a first nucleotide sequence encoding a signal peptide; d) a second nucleotide sequence encoding a first antigen, or an antigenic fragment thereof, from a wild-type SARS-CoV-2 virus or a first virus variant selected from the group consisting of: a Wuhan-Hu-1 variant, an Omicron variant, and a Delta variant; e) a third nucleotide sequence encoding a first linker; f) a fourth nucleotide sequence encoding a T4 foldon domain; g) a fifth nucleotide sequence encoding a second linker; h) a sixth nucleotide sequence encoding a second antigen, or an antigenic fragment thereof, from a wild-type SARS-CoV-2 virus or a second virus variant selected from the group consisting of: a Wuhan-Hu-1 variant, an Omicron variant, and a Delta variant; i) a 3′ UTR; and j) a poly A tail.

Provided herein, in certain embodiments, are purified RNA molecules comprising, from 5′ to 3′: a) a m7GpppNm-, where Nm denotes any nucleotide with a 2′ O methylation (Cap 1); b) a 5′ UTR; c) a first nucleotide sequence encoding a signal peptide; d) a second nucleotide sequence encoding a first antigen, or an antigenic fragment thereof, from an Omicron variant; e) a third nucleotide sequence encoding a first linker; f) a fourth nucleotide sequence encoding a T4 foldon domain; g) a fifth nucleotide sequence encoding a second linker; h) a sixth nucleotide sequence encoding a second antigen, or an antigenic fragment thereof, from a wild-type SARS-CoV-2 virus; i) a 3′ UTR; and j) a poly A tail.

Provided herein, in certain embodiments, are purified RNA molecules comprising, from 5′ to 3′: a) a m7GpppNm-, where Nm denotes any nucleotide with a 2′ O methylation (Cap 1); b) a 5′ UTR; c) a first nucleotide sequence encoding a signal peptide; d) a second nucleotide sequence encoding a first antigen, or an antigenic fragment thereof, from a wild-type SARS-CoV-2 virus; e) a third nucleotide sequence encoding a first linker; f) a fourth nucleotide sequence encoding a T4 foldon domain; g) a fifth nucleotide sequence encoding a second linker; h) a sixth nucleotide sequence encoding a second antigen, or an antigenic fragment thereof, from an Omicron variant; i) a 3′ UTR; and j) a poly A tail.

Provided herein, in certain embodiments, are purified RNA molecules comprising, from 5′ to 3′: a) a m7GpppNm-, where Nm denotes any nucleotide with a 2′ O methylation (Cap 1); b) a 5′ UTR; c) a first nucleotide sequence encoding a first antigen, or an antigenic fragment thereof, from an influenza virus selected from the group consisting of H1N1 (PR8), H2N2, H1N2, H3N2 (Udorn), N2-NA, Wyo03, PC73, H5N1. H5N2, H5N8, H5N9, H7N2, H7N3, H7N7, H9N2, H10N7, and H10N3; d) optionally, a second nucleotide sequence encoding a first linker; e) optionally, a third nucleotide sequence encoding a T4 foldon domain; f) optionally, a fourth nucleotide sequence encoding a second linker; g) optionally, a fifth nucleotide sequence encoding a second antigen, or an antigenic fragment thereof, from an influenza virus selected from the group consisting of H1N1 (PR8), H2N2, H1N2, H3N2 (Udorn), N2-NA, Wyo03, PC73, H5N1. H5N2, H5N8, H5N9, H7N2, H7N3, H7N7, H9N2, H10N7, and H10N3; h) a 3′ UTR; and i) a poly A tail.

Provided herein, in certain embodiments, are purified RNA molecules comprising, from 5′ to 3′: a) a m7GpppNm-, where Nm denotes any nucleotide with a 2′ O methylation (Cap 1); b) a 5′ UTR; c) a first nucleotide sequence encoding a first antigen, or an antigenic fragment thereof, from a H1N1 (PR8) influenza virus; d) a second nucleotide sequence encoding a first linker; e) a third nucleotide sequence encoding a T4 foldon domain; f) a fourth nucleotide sequence encoding a second linker; g) a fifth nucleotide sequence encoding a second antigen, or an antigenic fragment thereof, from a H3N2 (Udorn) influenza virus; h) a 3′ UTR; and i) a poly A tail.

Provided herein, in certain embodiments, are purified RNA molecules comprising, from 5′ to 3′: a) a m7GpppNm-, where Nm denotes any nucleotide with a 2′ O methylation (Cap 1); b) a 5′ UTR; c) a nucleotide sequence encoding a first antigen, or an antigenic fragment thereof, from a H1N1 (PR8) influenza virus; d) a 3′ UTR; and e) a poly A tail.

Provided herein, in certain embodiments, are purified RNA molecules comprising, from 5′ to 3′: a) a m7GpppNm-, where Nm denotes any nucleotide with a 2′ O methylation (Cap 1); b) a 5′ UTR; c) a nucleotide sequence encoding a first antigen, or an antigenic fragment thereof, from a H3N2 (Udorn) influenza virus; d) a 3′ UTR; and e) a poly A tail.

In some embodiments, the purified RNA molecule comprises a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 26. In some embodiments, the purified RNA molecule comprises a sequence having at least about 70% identity to SEQ ID NO: 26. In some embodiments, the purified RNA molecule comprises a sequence having at least about 75% identity to SEQ ID NO: 26. In some embodiments, the purified RNA molecule comprises a sequence having at least about 80% identity to SEQ ID NO: 26. In some embodiments, the purified RNA molecule comprises a sequence having at least about 85% identity to SEQ ID NO: 26. In some embodiments, the purified RNA molecule comprises a sequence having at least about 90% identity to SEQ ID NO: 26. In some embodiments, the purified RNA molecule comprises a sequence having at least about 95% identity to SEQ ID NO: 26. In some embodiments, the purified RNA molecule comprises a sequence having at least about 96% identity to SEQ ID NO: 26. In some embodiments, the purified RNA molecule comprises a sequence having at least about 97% identity to SEQ ID NO: 26. In some embodiments, the purified RNA molecule comprises a sequence having at least about 98% identity to SEQ ID NO: 26. In some embodiments, the purified RNA molecule comprises a sequence having at least about 99% identity to SEQ ID NO: 26. In some embodiments, the purified RNA molecule comprises a sequence having 100% identity to SEQ ID NO: 26.

In some embodiments, the purified RNA molecule comprises a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 27. In some embodiments, the purified RNA molecule comprises a sequence having at least about 70% identity to SEQ ID NO: 27. In some embodiments, the purified RNA molecule comprises a sequence having at least about 75% identity to SEQ ID NO: 27. In some embodiments, the purified RNA molecule comprises a sequence having at least about 80% identity to SEQ ID NO: 27. In some embodiments, the purified RNA molecule comprises a sequence having at least about 85% identity to SEQ ID NO: 27. In some embodiments, the purified RNA molecule comprises a sequence having at least about 90% identity to SEQ ID NO: 27. In some embodiments, the purified RNA molecule comprises a sequence having at least about 95% identity to SEQ ID NO: 27. In some embodiments, the purified RNA molecule comprises a sequence having at least about 96% identity to SEQ ID NO: 27. In some embodiments, the purified RNA molecule comprises a sequence having at least about 97% identity to SEQ ID NO: 27. In some embodiments, the purified RNA molecule comprises a sequence having at least about 98% identity to SEQ ID NO: 27. In some embodiments, the purified RNA molecule comprises a sequence having at least about 99% identity to SEQ ID NO: 27. In some embodiments, the purified RNA molecule comprises a sequence having 100% identity to SEQ ID NO: 27.

In some embodiments, the purified RNA molecule comprises a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 28. In some embodiments, the purified RNA molecule comprises a sequence having at least about 70% identity to SEQ ID NO: 28. In some embodiments, the purified RNA molecule comprises a sequence having at least about 75% identity to SEQ ID NO: 28. In some embodiments, the purified RNA molecule comprises a sequence having at least about 80% identity to SEQ ID NO: 28. In some embodiments, the purified RNA molecule comprises a sequence having at least about 85% identity to SEQ ID NO: 28. In some embodiments, the purified RNA molecule comprises a sequence having at least about 90% identity to SEQ ID NO: 28. In some embodiments, the purified RNA molecule comprises a sequence having at least about 95% identity to SEQ ID NO: 28. In some embodiments, the purified RNA molecule comprises a sequence having at least about 96% identity to SEQ ID NO: 28. In some embodiments, the purified RNA molecule comprises a sequence having at least about 97% identity to SEQ ID NO: 28. In some embodiments, the purified RNA molecule comprises a sequence having at least about 98% identity to SEQ ID NO: 28. In some embodiments, the purified RNA molecule comprises a sequence having at least about 99% identity to SEQ ID NO: 28. In some embodiments, the purified RNA molecule comprises a sequence having 100% identity to SEQ ID NO: 28.

In some embodiments, the purified RNA molecule comprises a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 29. In some embodiments, the purified RNA molecule comprises a sequence having at least about 70% identity to SEQ ID NO: 29. In some embodiments, the purified RNA molecule comprises a sequence having at least about 75% identity to SEQ ID NO: 29. In some embodiments, the purified RNA molecule comprises a sequence having at least about 80% identity to SEQ ID NO: 29. In some embodiments, the purified RNA molecule comprises a sequence having at least about 85% identity to SEQ ID NO: 29. In some embodiments, the purified RNA molecule comprises a sequence having at least about 90% identity to SEQ ID NO: 29. In some embodiments, the purified RNA molecule comprises a sequence having at least about 95% identity to SEQ ID NO: 29. In some embodiments, the purified RNA molecule comprises a sequence having at least about 96% identity to SEQ ID NO: 29. In some embodiments, the purified RNA molecule comprises a sequence having at least about 97% identity to SEQ ID NO: 29. In some embodiments, the purified RNA molecule comprises a sequence having at least about 98% identity to SEQ ID NO: 29. In some embodiments, the purified RNA molecule comprises a sequence having at least about 99% identity to SEQ ID NO: 29. In some embodiments, the purified RNA molecule comprises a sequence having 100% identity to SEQ ID NO: 29.

In some embodiments, the purified RNA molecule comprises a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 30. In some embodiments, the purified RNA molecule comprises a sequence having at least about 70% identity to SEQ ID NO: 30. In some embodiments, the purified RNA molecule comprises a sequence having at least about 75% identity to SEQ ID NO: 30. In some embodiments, the purified RNA molecule comprises a sequence having at least about 80% identity to SEQ ID NO: 30. In some embodiments, the purified RNA molecule comprises a sequence having at least about 85% identity to SEQ ID NO: 30. In some embodiments, the purified RNA molecule comprises a sequence having at least about 90% identity to SEQ ID NO: 30. In some embodiments, the purified RNA molecule comprises a sequence having at least about 95% identity to SEQ ID NO: 30. In some embodiments, the purified RNA molecule comprises a sequence having at least about 96% identity to SEQ ID NO: 30. In some embodiments, the purified RNA molecule comprises a sequence having at least about 97% identity to SEQ ID NO: 30. In some embodiments, the purified RNA molecule comprises a sequence having at least about 98% identity to SEQ ID NO: 30. In some embodiments, the purified RNA molecule comprises a sequence having at least about 99% identity to SEQ ID NO: 30. In some embodiments, the purified RNA molecule comprises a sequence having 100% identity to SEQ ID NO: 30.

In some embodiments, the purified RNA molecule comprises a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 31. In some embodiments, the purified RNA molecule comprises a sequence having at least about 70% identity to SEQ ID NO: 31. In some embodiments, the purified RNA molecule comprises a sequence having at least about 75% identity to SEQ ID NO: 31. In some embodiments, the purified RNA molecule comprises a sequence having at least about 80% identity to SEQ ID NO: 31. In some embodiments, the purified RNA molecule comprises a sequence having at least about 85% identity to SEQ ID NO: 31. In some embodiments, the purified RNA molecule comprises a sequence having at least about 90% identity to SEQ ID NO: 31. In some embodiments, the purified RNA molecule comprises a sequence having at least about 95% identity to SEQ ID NO: 31. In some embodiments, the purified RNA molecule comprises a sequence having at least about 96% identity to SEQ ID NO: 31. In some embodiments, the purified RNA molecule comprises a sequence having at least about 97% identity to SEQ ID NO: 31. In some embodiments, the purified RNA molecule comprises a sequence having at least about 98% identity to SEQ ID NO: 31. In some embodiments, the purified RNA molecule comprises a sequence having at least about 99% identity to SEQ ID NO: 31. In some embodiments, the purified RNA molecule comprises a sequence having 100% identity to SEQ ID NO: 31.

In some embodiments, the purified RNA molecule is encoded by a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 12. In some embodiments, the purified RNA molecule is encoded by a sequence having at least about 70% identity to SEQ ID NO: 12. In some embodiments, the purified RNA molecule is encoded by a sequence having at least about 75% identity to SEQ ID NO: 12. In some embodiments, the purified RNA molecule is encoded by a sequence having at least about 80% identity to SEQ ID NO: 12. In some embodiments, the purified RNA molecule is encoded by a sequence having at least about 85% identity to SEQ ID NO: 12. In some embodiments, the purified RNA molecule is encoded by a sequence having at least about 90% identity to SEQ ID NO: 12. In some embodiments, the purified RNA molecule is encoded by a sequence having at least about 95% identity to SEQ ID NO: 12. In some embodiments, the purified RNA molecule is encoded by a sequence having at least about 96% identity to SEQ ID NO: 12. In some embodiments, the purified RNA molecule is encoded by a sequence having at least about 97% identity to SEQ ID NO: 12. In some embodiments, the purified RNA molecule is encoded by a sequence having at least about 98% identity to SEQ ID NO: 12. In some embodiments, the purified RNA molecule is encoded by a sequence having at least about 99% identity to SEQ ID NO: 12. In some embodiments, the purified RNA molecule is encoded by a sequence having 100% identity to SEQ ID NO: 12.

In some embodiments, the purified RNA molecule is encoded by a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 13. In some embodiments, the purified RNA molecule is encoded by a sequence having at least about 70% identity to SEQ ID NO: 13. In some embodiments, the purified RNA molecule is encoded by a sequence having at least about 75% identity to SEQ ID NO: 13. In some embodiments, the purified RNA molecule is encoded by a sequence having at least about 80% identity to SEQ ID NO: 13. In some embodiments, the purified RNA molecule is encoded by a sequence having at least about 85% identity to SEQ ID NO: 13. In some embodiments, the purified RNA molecule is encoded by a sequence having at least about 90% identity to SEQ ID NO: 13. In some embodiments, the purified RNA molecule is encoded by a sequence having at least about 95% identity to SEQ ID NO: 13. In some embodiments, the purified RNA molecule is encoded by a sequence having at least about 96% identity to SEQ ID NO: 13. In some embodiments, the purified RNA molecule is encoded by a sequence having at least about 97% identity to SEQ ID NO: 13. In some embodiments, the purified RNA molecule is encoded by a sequence having at least about 98% identity to SEQ ID NO: 13. In some embodiments, the purified RNA molecule is encoded by a sequence having at least about 99% identity to SEQ ID NO: 13. In some embodiments, the purified RNA molecule is encoded by a sequence having 100% identity to SEQ ID NO: 13.

When transfected into mammalian cells, the modified RNAs typically have a stability of between 12-18 hours, or greater than 18 hours, e.g., 24, 36, 48, 60, 72, or greater than 72 hours.

Aside from mRNA, the RNA molecules described herein can be one of several non-coding types of RNA, such as a ribosomal RNA (rRNA) or a transfer RNA (tRNA). The terms “RNA” or “RNA molecule” further encompass other coding RNA molecules, such as viral RNA, retroviral RNA, self-replicating RNA (replicon RNA), small interfering RNA (siRNA), microRNA, small nuclear RNA (snRNA), small-hairpin (sh) RNA, riboswitches, ribozymes or aptamers.

In certain embodiments, the RNA molecule is a long RNA or a long RNA molecule. The term “long RNA” as used herein typically refers to an RNA molecule, preferably as described herein, which preferably comprises at least 30 nucleotides. Alternatively, a long RNA may comprise at least 35, 40, 45, 50, 55, 60, 65, 70, 75, 80, 85, 90, 95, 100, 150, 200, 250, 300, 350, 400, 450 or at least 500 nucleotides. A long RNA may comprise at least 1000 nucleotides or at least 2000 nucleotides. A long RNA, in the context of the present disclosure, may comprise from about 30 to about 50,000 nucleotides, from about 30 to about 20,000 nucleotides, from about 100 to about 20,000 nucleotides, from about 200 to about 20,000 nucleotides, from about 200 to about 15,000 nucleotides, from about 500 to about 20,000 nucleotides, from about 1,000 to about 15,000 nucleotides, from about 1,500 to about 10,000 nucleotides, from about 2,000 to about 5,000 nucleotides. The term “long RNA” as used herein is not limited to a certain type of RNA, but merely refers to the number of nucleotides contained in said RNA. In certain embodiments, the RNA as used herein is a long RNA.

In the present disclosure, the RNA molecule may be a coding RNA molecule encoding one or more proteins or peptides, which may be selected, without being restricted thereto, e.g., from therapeutically active proteins or peptides, selected from adjuvant proteins, from antigens, e.g., pathogenic antigens (e.g., animal antigens, from viral antigens, from protozoan antigens, from bacterial antigens), allergenic antigens, autoimmune antigens, or further antigens, preferably as defined herein, from allergens, from antibodies, from immunostimulatory proteins or peptides, from antigen-specific T-cell receptors, or from any other protein or peptide suitable for a specific (therapeutic) application, wherein the coding RNA molecule may be transported into a cell, a tissue or an organism, and the protein may be expressed subsequently in this cell, tissue or organism.

In certain embodiments, the RNA molecule of present disclosure is an immunostimulatory RNA molecule, which is capable of inducing an immune response, preferably an innate immune response. Such an immunostimulatory RNA may be any (double-stranded or single-stranded) RNA, e.g., a coding RNA, as defined herein. In certain embodiments, the immunostimulatory RNA is a non-coding RNA. The immunostimulatory RNA may be a single-stranded, a double-stranded, or a partially double-stranded RNA, optionally a single-stranded RNA or a circular or linear RNA, preferably, a linear RNA. In various embodiments, the immunostimulatory RNA may be a linear single-stranded RNA. Even more preferably, the immunostimulatory RNA may be a long, linear single-stranded RNA.

An immunostimulatory RNA may also occur as a short RNA oligonucleotide. As used herein, an immunostimulatory RNA may be selected from any class of RNA molecules, found in nature or being prepared synthetically, and which can induce an innate immune response and may support an adaptive immune response induced by an antigen.

In some embodiments, the RNA molecule is a self-replicating RNA.

A self-replicating RNA molecule (replicon) can, when delivered to a vertebrate cell even without any proteins, lead to the production of multiple daughter RNAs by transcription from itself (via an antisense copy which it generates from itself). A self-replicating RNA molecule can thus typically be a +-strand molecule which can be directly translated after delivery to a cell, and this translation provides an RNA-dependent RNA polymerase which then produces both antisense and sense transcripts from the delivered RNA. Thus the delivered RNA can lead to the production of multiple daughter RNAs. These daughter RNAs, as well as collinear subgenomic transcripts, may be translated themselves to provide in situ expression of an encoded immunogen, or may be transcribed to provide further transcripts with the same sense as the delivered RNA which are translated to provide in situ expression of the immunogen. The overall results of this sequence of transcriptions are a large amplification in the number of the introduced replicon RNAs and so the encoded immunogen becomes a major polypeptide product of the cells.

One suitable system for achieving self-replication in this manner is to use an alphavirus-based replicon. These replicons can be +-stranded RNAs which lead to translation of a replicase (or replicase-transcriptase) after delivery to a cell. The replicase can be translated as a polyprotein which auto-cleaves to provide a replication complex which creates genomic—strand copies of the +-strand delivered RNA. These—strand transcripts can themselves be transcribed to give further copies of the +-stranded parent RNA and also to give a subgenomic transcript which encodes the immunogen. Translation of the subgenomic transcript can thus lead to in situ expression of the immunogen by the infected cell. Suitable alphavirus replicons can use a replicase from a Sindbis virus, a SemLiki forest virus, an eastern equine encephalitis virus, a Venezuelan equine encephalitis virus, etc. Mutant or wild-type virus sequences can be used e.g., the attenuated TC83 mutant of VEEV has been used in replicons.

A self-replicating RNA molecule can encode (i) an RNA-dependent RNA polymerase which can transcribe RNA from the self-replicating RNA molecule and (ii) an immunogen. The polymerase can be an alphavirus replicase e.g., comprising one or more of alphavirus proteins nsp1, nsp2, nsp3 and nsp4.

Whereas natural alphavirus genomes encode structural virion proteins in addition to the non-structural replicase polyprotein, a self-replicating RNA molecule described herein can lack one or more or all alphavirus structural proteins. Thus a self-replicating RNA can lead to the production of genomic RNA copies of itself in a cell, but not to the production of RNA-containing virions. The inability to produce these virions means that, unlike a wild-type alphavirus, the self-replicating RNA molecule generally does not perpetuate itself in infectious form. The alphavirus structural proteins which are used for perpetuation in wild-type viruses are typically absent from self-replicating RNAs described herein and their place is taken by gene(s) encoding the immunogen of interest, such that the subgenomic transcript encodes the immunogen rather than the structural alphavirus virion proteins.

Thus a self-replicating RNA molecule may have two open reading frames. The first (5′) open reading frame encodes a replicase; the second (3′) open reading frame encodes an immunogen. In some embodiments the RNA has additional (e.g., downstream) open reading frames, e.g., to encode further immunogens (see below) or to encode accessory polypeptides.

Self-replicating RNA molecules can have various lengths but they are typically 5,000-25,000 nucleotides long, e.g., 8,000-15,000 nucleotides, or 9,000-12,000 nucleotides.

A self-replicating RNA molecule described herein may have a 5′ cap (e.g., a 7-methylguanosine). This cap can enhance in vivo translation of the RNA. The 5′ nucleotide of a RNA molecule useful with the present disclosure may have a 5′ triphosphate group. In a capped RNA this may be linked to a 7-methylguanosine via a 5′-to-5′ bridge. In some embodiments, the RNA cap includes m7G (Cap 0), m7GpppNm-, where Nm denotes any nucleotide with a 2′ O methylation (Cap 1), N6,2′-O-dimethyladenosine (m6AM), m7G(5′)ppp(5′) G (mCAP), or anti-reverse cap analogs (ARCA), optionally m7G or m7GpppNm—where Nm denotes any nucleotide with a 2′ O methylation.

A self-replicating RNA molecule may have a 3′ poly-A tail. It may also include a poly-A polymerase recognition sequence (e.g., AAUAAA) near its 3′ end.

A self-replicating RNA molecule will typically be single-stranded. Single-stranded RNAs can generally initiate an adjuvant effect by binding to TLR7, TLR8, RNA helicases and/or PKR. RNA delivered in double-stranded form (dsRNA) can bind to TLR3, and this receptor can also be triggered by dsRNA which is formed either during replication of a single-stranded RNA or within the secondary structure of a single-stranded RNA.

A self-replicating RNA molecule described herein can be prepared by in vitro transcription (IVT). IVT can use a cDNA template created and propagated in plasmid form in bacteria, or created synthetically (for example, by gene synthesis and/or polymerase chain-reaction (PCR) engineering methods). For instance, a DNA-dependent RNA polymerase (such as the bacteriophage T7, T3 or SP6 RNA polymerases) can be used to transcribe the RNA from a DNA template. Appropriate capping and poly-A addition reactions can be used as required (although the replicon's poly-A is usually encoded within the DNA template). These RNA polymerases can have stringent requirements for the transcribed 5′ nucleotide(s) and, in some embodiments, these requirements must be matched with the requirements of the encoded replicase, to ensure that the IVT-transcribed RNA can function efficiently as a substrate for its self-encoded replicase.

In some embodiments, the RNA molecule is not a self-replicating RNA.

In some embodiments, the RNA of the present disclosure comprises a purified RNA molecule, such as a messenger RNA (mRNA). RNA, for example, is transcribed in vitro from template DNA, referred to as an “in vitro transcription template.”

In vitro transcription of RNA is known in the art and is described, e.g., in International Publication WO2014/152027, which is incorporated by reference herein in its entirety. For example, in some embodiments, the RNA transcript is generated using a non-amplified, linearized DNA template in an in vitro transcription reaction to generate the RNA transcript. In some embodiments the RNA transcript is capped via enzymatic capping. In some embodiments, the RNA transcript is purified via chromatographic methods, e.g., use of an oligo dT substrate. Some embodiments exclude the use of DNase. In some embodiments the RNA transcript is synthesized from a non-amplified, linear DNA template coding for the gene of interest via an enzymatic in vitro transcription reaction utilizing a T7 phage RNA polymerase and nucleotide triphosphates of the desired chemistry. Any number of RNA polymerases or variants may be used in the method of the present disclosure. The polymerase may be selected from, but is not limited to, a phage RNA polymerase, e.g., a T7 RNA polymerase, a T3 RNA polymerase, a SP6 RNA polymerase, and/or mutant polymerases such as, but not limited to, polymerases able to incorporate modified nucleic acids and/or modified nucleotides, including chemically modified nucleic acids and/or nucleotides.

In some embodiments, an in vitro transcription template encodes a 5′ untranslated (UTR) region, contains an open reading frame, and encodes a 3′ UTR and a polyA tail. The particular nucleic acid sequence composition and length of an in vitro transcription template will depend on the RNA encoded by the template.

### Antigens

The RNA molecules described herein can be designed to contain or encode for a substance that produces an immune response in a subject. After administration of the RNA molecule, the RNA is translated in vivo. The RNA may elicit an immune response against an antigen, virus, and/or viral antigen. The immune response may comprise an antibody response. The RNA will typically elicit an immune response which recognizes the corresponding antigen such as a viral polypeptide. The RNA will typically comprise one or more sequences of a surface polypeptide, e.g., an adhesin, a hemagglutinin, an envelope glycoprotein, a spike glycoprotein, a receptor-binding domain (RBD). In some embodiments, the RNA elicits an immune response against receptor binding domains (RBDs) of SARS-CoV-2.

In some embodiments, the RNA molecules provided herein have a first nucleotide sequence encoding a first antigen, or an antigenic fragment thereof, from a first virus variant, a second nucleotide sequence encoding a multimerization domain, and a third nucleotide sequence encoding a second antigen, or an antigenic fragment thereof, from a second virus variant, wherein the first virus variant and the second virus variant are different, and wherein the first nucleotide sequence, the second nucleotide sequence, and the third nucleotide sequence are operably linked to each other in a 5′-to-3′ direction.

In some embodiments, the virus is selected from Orthomyxoviruses, such as influenza virus; Rhabdoviruses, such as rabies virus; Picornaviruses, such as Poliovirus; Poxviruses, such as vaccinia virus; Rotavirus; respiratory syncytial virus (RSV), and coronaviruses, such as COVID-19.

In some embodiments, the virus is selected from the group consisting of 229E (alpha coronavirus), NL63 (alpha coronavirus), OC43 (beta coronavirus), HKU1 (beta coronavirus), MERS-CoV (MERS), SARS-CoV (SARS), and SARS-CoV-2 (COVID-19). In some embodiments, the virus is SARS-CoV-2.

In some embodiments, the first virus variant or the second virus variant is selected from the group consisting of Wuhan-Hu-1, Alpha, Beta, Gamma, Delta, Epsilon Eta, Iota, Kappa, 1.617.3, Mu, Zeta, and Omicron. In some embodiments, the first virus variant or the second virus variant is selected from the group consisting of Wuhan-Hu-1 and Delta.

In some embodiments, the first antigen, or the antigenic fragment thereof, or the second antigen, or the antigenic fragment thereof, comprises a viral envelope protein, viral spike protein, viral membrane protein, or viral capsid protein. In some embodiments, the first antigen, or the antigenic fragment thereof, or the second antigen, or the antigenic fragment thereof, comprises a viral spike protein.

In some embodiments, the first antigen, or the antigenic fragment thereof, or the second antigen, or the antigenic fragment thereof, comprises a receptor-binding domain (RBD). In some embodiments, the RBD domain is a RBD domain from Wuhan-Hu-1 variant or Delta variant. In some embodiments, the first antigen, or the antigenic fragment thereof, comprises the RBD domain from the Delta variant and the second antigen, or the antigenic fragment thereof, comprises the RBD domain of the Wuhan-Hu-1 variant.

In some embodiments, the virus is an influenza A virus. In some embodiments, the influenza A virus is selected from the group consisting of H1N1 (PR8), H2N2, H1N2, H3N2 (Udorn), N2-NA, Wyo03, PC73, H5N1. H5N2, H5N8, H5N9, H7N2, H7N3, H7N7, H9N2, H10N7, and H10N3.

In some embodiments, the first virus variant or the second virus variant is selected from the group consisting of H1N1 (PR8) and H3N2 (Udorn).

In some embodiments, the first antigen, or the antigenic fragment thereof, or the second antigen, or the antigenic fragment thereof, comprises an RNA polymerase subunit, hemagglutinin (HA), nucleoprotein (NP), neuraminidase (NA), matrix protein 1 (M1), matrix protein 2 (M2), non-structural protein NS1, or non-structural protein NEP.

In some embodiments, the first antigen, or the antigenic fragment thereof, or the second antigen, or the antigenic fragment thereof, comprises a hemagglutinin (HA) protein.

In some embodiments, the HA protein is from H1N1 (PR8) or H3N2 (Udorn).

In some embodiments, the first antigen, or the antigenic fragment thereof, comprises an HA protein from H1N1 (PR8) and the second antigen, or the antigenic fragment thereof, comprises an HA protein from H3N2 (Udorn).

In some embodiments, the first antigen, or the antigenic fragment thereof, is encoded by a nucleotide sequence having at least 90% sequence identity to the sequence of any one of SEQ ID NOs: 3-4, 7, and 8. In some embodiments, the first antigen, or the antigenic fragment thereof is encoded by a nucleotide sequence having at least 70% (e.g., 75%, 80%, 90%, 95%, 97%, 98%, or 99%) sequence identity to the nucleic acid sequence of any one of SEQ ID NOs: 3-4, 7, and 8. In some embodiments, the first antigen, or the antigenic fragment thereof is encoded by a nucleotide sequence having at least 70% sequence identity to the nucleic acid sequence of any one of SEQ ID NOs: 3-4, 7, and 8. In some embodiments, the first antigen, or the antigenic fragment thereof is encoded by a nucleotide sequence having at least 80% sequence identity to the nucleic acid sequence of any one of SEQ ID NOs: 3-4, 7, and 8. In some embodiments, the first antigen, or the antigenic fragment thereof is encoded by a nucleotide sequence having at least 90% sequence identity to the nucleic acid sequence of any one of SEQ ID NOs: 3-4, 7, and 8. In some embodiments, the first antigen, or the antigenic fragment thereof is encoded by a nucleotide sequence having at least 95% sequence identity to the nucleic acid sequence of any one of SEQ ID NOs: 3-4, 7, and 8. In some embodiments, the first antigen, or the antigenic fragment thereof is encoded by a nucleotide sequence having at least 97% sequence identity to the nucleic acid sequence of any one of SEQ ID NOs: 3-4, 7, and 8. In some embodiments, the first antigen, or the antigenic fragment thereof is encoded by a nucleotide sequence having at least 98% sequence identity to the nucleic acid sequence of any one of SEQ ID NOs: 3-4, 7, and 8. In some embodiments, the first antigen, or the antigenic fragment thereof is encoded by a nucleotide sequence having at least 99% sequence identity to the nucleic acid sequence of any one of SEQ ID NOs: 3-4, 7, and 8. In some embodiments, the first antigen, or the antigenic fragment thereof is encoded by a nucleotide sequence having the nucleic acid sequence of any one of SEQ ID NOs: 3-4, 7, and 8.

In some embodiments, the first antigen, or the antigenic fragment thereof, comprises sequence having at least 90% sequence identity to the sequence of any one of SEQ ID NOs: 19, 20, 22, and 23. In some embodiments, the first antigen, or the antigenic fragment thereof comprises sequence having at least 70% (e.g., 75%, 80%, 90%, 95%, 97%, 98%, or 99%) sequence identity to the nucleic acid sequence of any one of SEQ ID NOs: 19, 20, 22, and 23. In some embodiments, the first antigen, or the antigenic fragment thereof comprises sequence having at least 70% sequence identity to the nucleic acid sequence of any one of SEQ ID NOs: 19, 20, 22, and 23. In some embodiments, the first antigen, or the antigenic fragment thereof comprises sequence having at least 80% sequence identity to the nucleic acid sequence of any one of SEQ ID NOs: 19, 20, 22, and 23. In some embodiments, the first antigen, or the antigenic fragment thereof comprises sequence having at least 90% sequence identity to the nucleic acid sequence of any one of SEQ ID NOs: 19, 20, 22, and 23. In some embodiments, the first antigen, or the antigenic fragment thereof comprises sequence having at least 95% sequence identity to the nucleic acid sequence of any one of SEQ ID NOs: 19, 20, 22, and 23. In some embodiments, the first antigen, or the antigenic fragment thereof comprises sequence having at least 97% sequence identity to the nucleic acid sequence of any one of SEQ ID NOs: 19, 20, 22, and 23. In some embodiments, the first antigen, or the antigenic fragment thereof comprises sequence having at least 98% sequence identity to the nucleic acid sequence of any one of SEQ ID NOs: 19, 20, 22, and 23. In some embodiments, the first antigen, or the antigenic fragment thereof comprises sequence having at least 99% sequence identity to the nucleic acid sequence of any one of SEQ ID NOs: 19, 20, 22, and 23. In some embodiments, the first antigen, or the antigenic fragment thereof comprises sequence having the nucleic acid sequence of any one of SEQ ID NOs: 19, 20, 22, and 23.

In some embodiments, the second antigen, or the antigenic fragment thereof is encoded by a nucleotide sequence having at least 70% (e.g., 75%, 80%, 90%, 95%, 97%, 98%, or 99%) sequence identity to the nucleic acid sequence of any one of SEQ ID NOs: 3-4, 7, and 8. In some embodiments, the second antigen, or the antigenic fragment thereof is encoded by a nucleotide sequence having at least 70% sequence identity to the nucleic acid sequence of any one of SEQ ID NOs: 3-4, 7, and 8. In some embodiments, the second antigen, or the antigenic fragment thereof is encoded by a nucleotide sequence having at least 80% sequence identity to the nucleic acid sequence of any one of SEQ ID NOs: 3-4, 7, and 8. In some embodiments, the second antigen, or the antigenic fragment thereof is encoded by a nucleotide sequence having at least 90% sequence identity to the nucleic acid sequence of any one of SEQ ID NOs: 3-4, 7, and 8. In some embodiments, the second antigen, or the antigenic fragment thereof is encoded by a nucleotide sequence having at least 95% sequence identity to the nucleic acid sequence of any one of SEQ ID NOs: 3-4, 7, and 8. In some embodiments, the second antigen, or the antigenic fragment thereof is encoded by a nucleotide sequence having at least 97% sequence identity to the nucleic acid sequence of any one of SEQ ID NOs: 3-4, 7, and 8. In some embodiments, the second antigen, or the antigenic fragment thereof is encoded by a nucleotide sequence having at least 98% sequence identity to the nucleic acid sequence of any one of SEQ ID NOs: 3-4, 7, and 8. In some embodiments, the second antigen, or the antigenic fragment thereof is encoded by a nucleotide sequence having at least 99% sequence identity to the nucleic acid sequence of any one of SEQ ID NOs: 3-4, 7, and 8. In some embodiments, the second antigen, or the antigenic fragment thereof is encoded by a nucleotide sequence having the nucleic acid sequence of any one of SEQ ID NOs: 3-4, 7, and 8.

In some embodiments, the second antigen, or the antigenic fragment thereof, comprises sequence having at least 90% sequence identity to the sequence of any one of SEQ ID NOs: 19, 20, 22, and 23. In some embodiments, the second antigen, or the antigenic fragment thereof comprises sequence having at least 70% (e.g., 75%, 80%, 90%, 95%, 97%, 98%, or 99%) sequence identity to the nucleic acid sequence of any one of SEQ ID NOs: 19, 20, 22, and 23. In some embodiments, the second antigen, or the antigenic fragment thereof comprises sequence having at least 70% sequence identity to the nucleic acid sequence of any one of SEQ ID NOs: 19, 20, 22, and 23. In some embodiments, the second antigen, or the antigenic fragment thereof comprises sequence having at least 80% sequence identity to the nucleic acid sequence of any one of SEQ ID NOs: 19, 20, 22, and 23. In some embodiments, the second antigen, or the antigenic fragment thereof comprises sequence having at least 90% sequence identity to the nucleic acid sequence of any one of SEQ ID NOs: 19, 20, 22, and 23. In some embodiments, the second antigen, or the antigenic fragment thereof comprises sequence having at least 95% sequence identity to the nucleic acid sequence of any one of SEQ ID NOs: 19, 20, 22, and 23. In some embodiments, the second antigen, or the antigenic fragment thereof comprises sequence having at least 97% sequence identity to the nucleic acid sequence of any one of SEQ ID NOs: 19, 20, 22, and 23. In some embodiments, the second antigen, or the antigenic fragment thereof comprises sequence having at least 98% sequence identity to the nucleic acid sequence of any one of SEQ ID NOs: 19, 20, 22, and 23. In some embodiments, the second antigen, or the antigenic fragment thereof comprises sequence having at least 99% sequence identity to the nucleic acid sequence of any one of SEQ ID NOs: 19, 20, 22, and 23. In some embodiments, the second antigen, or the antigenic fragment thereof comprises sequence having the nucleic acid sequence of any one of SEQ ID NOs: 19, 20, 22, and 23.

In some embodiments, the first antigen sequence is encoded by a portion of the nucleic acid sequence any one of SEQ ID NOs: 3-4, 7, and 8. In some embodiments, the first antigen sequence is encoded by at least 50, 52, 55, 57, 60, 62, 65, 67, or more than 70 nucleotides of the nucleic acid sequence any one of SEQ ID NOs: 3-4, 7, and 8. In some embodiments, the first antigen sequence is encoded by at least 50, 52, 55, 57, 60, 62, 65, 67, or more than 70 consecutive nucleotides of the nucleic acid sequence any one of SEQ ID NOs: 3-4, 7, and 8. In some embodiments, the first antigen sequence comprises about 50 nucleotides to about 72 nucleotides. In some embodiments, the first antigen sequence is encoded by at least about 50 nucleotides. In some embodiments, the first antigen sequence comprises at most about 72 nucleotides. In some embodiments, the first antigen sequence comprises about 50 nucleotides to about 70 nucleotides, about 50 nucleotides to about 67 nucleotides, about 50 nucleotides to about 65 nucleotides, about 50 nucleotides to about 62 nucleotides, about 50 nucleotides to about 60 nucleotides, about 50 nucleotides to about 57 nucleotides, about 50 nucleotides to about 55 nucleotides, or about 50 nucleotides to about 52 nucleotides of the nucleic acid sequence any one of SEQ ID NOs: 3-4, 7, and 8.

In some embodiments, the first antigen sequence comprises a portion of the nucleic acid sequence any one of SEQ ID NOs: 19, 20, 22, and 23. In some embodiments, the first antigen sequence comprises at least 50, 52, 55, 57, 60, 62, 65, 67, or more than 70 nucleotides of the nucleic acid sequence any one of SEQ ID NOs: 19, 20, 22, and 23. In some embodiments, the first antigen sequence comprises at least 50, 52, 55, 57, 60, 62, 65, 67, or more than 70 consecutive nucleotides of the nucleic acid sequence any one of SEQ ID NOS: 19, 20, 22, and 23. In some embodiments, the first antigen sequence comprises about 50 nucleotides to about 72 nucleotides. In some embodiments, the first antigen sequence comprises at least about 50 nucleotides. In some embodiments, the first antigen sequence comprises at most about 72 nucleotides. In some embodiments, the first antigen sequence comprises about 50 nucleotides to about 70 nucleotides, about 50 nucleotides to about 67 nucleotides, about 50 nucleotides to about 65 nucleotides, about 50 nucleotides to about 62 nucleotides, about 50 nucleotides to about 60 nucleotides, about 50 nucleotides to about 57 nucleotides, about 50 nucleotides to about 55 nucleotides, or about 50 nucleotides to about 52 nucleotides of the nucleic acid sequence any one of SEQ ID NOs: 19, 20, 22, and 23.

In some embodiments, the second antigen sequence is encoded by a portion of the nucleic acid sequence any one of SEQ ID NOs: 3-4, 7, and 8. In some embodiments, the second antigen sequence is encoded by at least 50, 52, 55, 57, 60, 62, 65, 67, or more than 70 nucleotides of the nucleic acid sequence any one of SEQ ID NOs: 3-4, 7, and 8. In some embodiments, the second antigen sequence is encoded by at least 50, 52, 55, 57, 60, 62, 65, 67, or more than 70 consecutive nucleotides of the nucleic acid sequence any one of SEQ ID NOs: 3-4, 7, and 8. In some embodiments, the second antigen sequence comprises about 50 nucleotides to about 72 nucleotides. In some embodiments, the second antigen sequence is encoded by at least about 50 nucleotides. In some embodiments, the second antigen sequence comprises at most about 72 nucleotides. In some embodiments, the second antigen sequence comprises about 50 nucleotides to about 70 nucleotides, about 50 nucleotides to about 67 nucleotides, about 50 nucleotides to about 65 nucleotides, about 50 nucleotides to about 62 nucleotides, about 50 nucleotides to about 60 nucleotides, about 50 nucleotides to about 57 nucleotides, about 50 nucleotides to about 55 nucleotides, or about 50 nucleotides to about 52 nucleotides of the nucleic acid sequence any one of SEQ ID NOs: 3-4, 7, and 8.

In some embodiments, the second antigen sequence comprises a portion of the nucleic acid sequence any one of SEQ ID NOs: 19, 20, 22, and 23. In some embodiments, the second antigen sequence comprises at least 50, 52, 55, 57, 60, 62, 65, 67, or more than 70 nucleotides of the nucleic acid sequence any one of SEQ ID NOs: 19, 20, 22, and 23. In some embodiments, the second antigen sequence comprises at least 50, 52, 55, 57, 60, 62, 65, 67, or more than 70 consecutive nucleotides of the nucleic acid sequence any one of SEQ ID NOs: 19, 20, 22, and 23. In some embodiments, the second antigen sequence comprises about 50 nucleotides to about 72 nucleotides. In some embodiments, the second antigen sequence comprises at least about 50 nucleotides. In some embodiments, the second antigen sequence comprises at most about 72 nucleotides. In some embodiments, the second antigen sequence comprises about 50 nucleotides to about 70 nucleotides, about 50 nucleotides to about 67 nucleotides, about 50 nucleotides to about 65 nucleotides, about 50 nucleotides to about 62 nucleotides, about 50 nucleotides to about 60 nucleotides, about 50 nucleotides to about 57 nucleotides, about 50 nucleotides to about 55 nucleotides, or about 50 nucleotides to about 52 nucleotides of the nucleic acid sequence any one of SEQ ID NOs: 19, 20, 22, and 23.

### Linker Sequences

Linker sequences can be used in a RNA molecule to separate different components of the RNA molecule described herein. The linker sequence can join two antigens, or antigenic fragments thereof or an antigen and an oligomerization domain. It is understood that the linker sequence is not a sequence that naturally separates a first and second RNA molecule, if the first and second RNA molecule happen to naturally exist in combination together.

In some embodiments, the RNA molecule described herein comprises a sequence encoding a first linker connecting the first antigen, or the antigenic fragment thereof, to a multimerization domain. In some embodiments, the RNA molecule described herein comprises a sequence encoding a second linker connecting the second antigen, or the antigenic fragment thereof, to a multimerization domain.

In some embodiments, the first linker encodes an amino acid sequence comprising at least 5 to about 50 amino acids. In some embodiments, one or both of the first linker and the second linker encodes an amino acid sequence comprising about 5 to about 50 amino acids, about 5 to about 45 amino acids, about 5 to about 40 amino acids, about 5 to about 35 amino acids, about 5 to about 30 amino acids, about 5 to about 25 amino acids, about 5 to about 20 amino acids, about 5 to about 15 amino acids, about 5 to about 10 amino acids, about 10 to about 50 amino acids, about 15 to about 50 amino acids, about 20 to about 50 amino acids, about 25 to about 50 amino acids, about 30 to about 50 amino acids, about 35 to about 50 amino acids, about 40 to about 50 amino acids, or about 45 to about 50 amino acids.

In some embodiments, the second linker encodes an amino acid sequence comprising at least 5 to about 50 amino acids. In some embodiments, one or both of the first linker and the second linker encodes an amino acid sequence comprising about 5 to about 50 amino acids, about 5 to about 45 amino acids, about 5 to about 40 amino acids, about 5 to about 35 amino acids, about 5 to about 30 amino acids, about 5 to about 25 amino acids, about 5 to about 20 amino acids, about 5 to about 15 amino acids, about 5 to about 10 amino acids, about 10 to about 50 amino acids, about 15 to about 50 amino acids, about 20 to about 50 amino acids, about 25 to about 50 amino acids, about 30 to about 50 amino acids, about 35 to about 50 amino acids, about 40 to about 50 amino acids, or about 45 to about 50 amino acids.

In some embodiments, the first linker encodes the amino acid sequence selected from the group consisting of (GS)n (SEQ ID NO: 9), (G2S)n (SEQ ID NO: 10), (G3S)n (SEQ ID NO: 11), (G4S)n (SEQ ID NO: 32), and (G)n (SEQ ID NO: 33), and wherein n is an integer from 2 to 20. In some embodiments, n is an integer from 2 to 18, from 2 to 16, from 2 to 14, from 2 to 12, from 2 to 10, from 2 to 8, from 2 to 6, from 2 to 4, from 4 to 20, from 6 to 20, from 8 to 20, from 10 to 20, from 12 to 20, from 14 to 20, from 16 to 20, or from 18 to 20.

In some embodiments, the second linker encodes the amino acid sequence selected from the group consisting of (GS)n (SEQ ID NO: 9), (G2S)n (SEQ ID NO: 10), (G3S)n (SEQ ID NO: 11), (G4S)n (SEQ ID NO: 32), and (G)n (SEQ ID NO: 33), and wherein n is an integer from 2 to 20. In some embodiments, n is an integer from 2 to 18, from 2 to 16, from 2 to 14, from 2 to 12, from 2 to 10, from 2 to 8, from 2 to 6, from 2 to 4, from 4 to 20, from 6 to 20, from 8 to 20, from 10 to 20, from 12 to 20, from 14 to 20, from 16 to 20, or from 18 to 20.

In some embodiments, the first linker encodes the amino acid sequence selected from the group consisting of (GGSGGD)n (SEQ ID NO: 34) or (GGSGGE)n (SEQ ID NO: 35), and wherein n is an integer from 2 to 6.

In some embodiments, the second linker encodes the amino acid sequence selected from the group consisting of (GGSGGD)n (SEQ ID NO: 34) or (GGSGGE)n (SEQ ID NO: 35), and wherein n is an integer from 2 to 6.

In some embodiments, the first linker encodes the amino acid sequence selected from the group consisting of (GGGSGSGGGGS)n (SEQ ID NO: 36) and (GGGGGPGGGGP) n (SEQ ID NO: 37), and wherein n is an integer from 1 to 3.

In some embodiments, one or both of the first linker and the second linker encodes the amino acid sequence selected from the group consisting of (GGGSGSGGGGS)n (SEQ ID NO: 36) and (GGGGGPGGGGP)n (SEQ ID NO: 37), and wherein n is an integer from 1 to 3.

In some embodiments, the first linker encodes the amino acid sequence selected from the group consisting of (GX)n, (GGX)n, (GGGX)n, (GGGGX)n, and (GzX)n, wherein z is between 1 and 20, and wherein n is at least 8. In some embodiments, z is between 2 and 18, 2 and 16, 2 and 14, 2 and 12, 2 and 10, 2 and 8, 2 and 6, 2 and 4, 4 and 20, 6 and 20, 8 and 20, 10 and 20, 12 and 20, 14 and 20, 16 and 20, or 18 and 20. In some embodiments, X is serine, aspartic acid, glutamic acid, threonine, or proline.

In some embodiments, the second linker encodes the amino acid sequence selected from the group consisting of (GX)n, (GGX)n, (GGGX)n, (GGGGX)n, and (GzX)n, wherein z is between 1 and 20, and wherein n is at least 8. In some embodiments, z is between 2 and 18, 2 and 16, 2 and 14, 2 and 12, 2 and 10, 2 and 8, 2 and 6, 2 and 4, 4 and 20, 6 and 20, 8 and 20, 10 and 20, 12 and 20, 14 and 20, 16 and 20, or 18 and 20. In some embodiments, X is serine, aspartic acid, glutamic acid, threonine, or proline.

In some embodiments, the first linker encodes GGSG (SEQ ID NO: 38). In some embodiments, the second linker encodes GGSG (SEQ ID NO: 38).

In some embodiments, first linker encodes GGSLGGGGSGS (SEQ ID NO: 39). In some embodiments, the second linker encodes GGSLGGGGSGS (SEQ ID NO: 39).

In some embodiments, the first linker is encoded by the nucleotide sequence according to SEQ ID NO: 5 or SEQ ID NO: 6.

In some embodiments, the second linker is encoded by the nucleotide sequence according to SEQ ID NO: 5 or SEQ ID NO: 6.

In some embodiments, the first linker is encoded by the nucleotide sequence according to SEQ ID NO: 5 and the second linker is encoded by the nucleotide sequence according to SEQ ID NO: 6. In some embodiments, the first linker is encoded by the nucleotide sequence according to SEQ ID NO: 6 and the second linker is encoded by the nucleotide sequence according to SEQ ID NO: 5. In some embodiments, the first linker is encoded by the nucleotide sequence according to SEQ ID NO: 5 and the second linker is encoded by the nucleotide sequence according to SEQ ID NO: 5. In some embodiments, the first linker is encoded by the nucleotide sequence according to SEQ ID NO: 6 and the second linker is encoded by the nucleotide sequence according to SEQ ID NO: 6.

### Fusion Proteins

The present disclosure relates generally to a strategy for multimerizing protein antigens in vaccine-related or other immunotherapeutic constructs. The strategy, in some embodiments, involves creating nucleic acid constructs with oligomerization motifs and a linker sequence separating two or more antigens such that the encoded fusion protein can form a dimeric, trimeric, tetrameric, hexameric, heptameric, or octameric complex from a single nucleic acid construct.

This strategy for multimerizing proteins can be exploited with proteins including viral, bacterial, parasitic, autoimmune, and tumor antigens. This platform can be used to create multimeric fusion proteins comprising multiple copies of a single antigen of interest. For example, a homodimer, homotrimer or tetramer can be created using two, three, or four copies of the same antigen with a dimerization, trimerization or tetramerization domain. When the oligomerization domains associate together, the construct will form a tetramer (if a dimerization domain is used) comprising four copies of the same antigen, a hexamer (if a trimerization domain is used) comprising six copies of the same antigen, or an octamer comprising eight copies of the same antigen (if a tetramerization domain is used).

Alternatively, this platform can be used to create multimeric fusion proteins comprising two or more different antigens of interest. For example, a heterodimer can be created with a first antigen linked to a second different, antigen (or a heterotrimer comprising two or three different antigens). When the oligomerization domains associate together, the construct will form a tetramer (if a dimerization domain is used) that is dimeric for both the first and second antigen, a hexamer (if a trimerization domain is used in the construct) that is dimeric for at least the first and second antigen or trimeric for the first, second, and third antigen, or an octamer (if a tetramerization domain is used). Alternatively a trimeric protein can be formed if the original protein is presented in monomeric form in association with the trimerization domain.

One aspect described herein is directed to a RNA comprising a first antigen, an oligomerization, or multimerization, domain, and a second antigen. One aspect described herein is directed to a RNA comprising a first antigen, a first linker sequence, an oligomerization domain, a second linker sequence, and a second antigen, wherein the first linker sequence joins the first antigen to the oligomerization domain, and the second linker joins the oligomerization domain to the second antigen. In one embodiment, the first and second antigens are the same. In another embodiment, the first and second antigens are different. The first and second antigens can be viral antigens, bacterial antigens, parasite antigens, autoimmune antigens, or tumor antigens. In one embodiment, the first and second antigens comprise a polypeptide and/or a polysaccharide. In one embodiment, the RNA forms a multimeric protein when expressed in a host cell. In another embodiment, the first and second antigens do not occur naturally as a multimeric protein.

In one embodiment, the RNA forms a multimeric protein when expressed in a host cell. In another embodiment, the first and second antigens do not occur naturally as a multimeric protein.

In some embodiments, the oligomerization or multimerization domain is selected from the group consisting of a dimerization domain, trimerization domain, and a tetramerization domain. In some embodiments, the oligomerization domain is a dimerization domain. In other embodiments, the oligomerization domain is a trimerization. In some embodiments, the oligomerization domain is a tetrameric domain.

In some embodiments, the multimerization domain is selected from the group consisting of enterobacteria phage T4, GCN4pII, GCN4-pLI, and p53. In some embodiments, the multimerization domain comprises a leucine zipper or a fibritin foldon domain. In some embodiments, the dimerization domain is a leucine zipper domain, including but not limited to a yeast GCN4 leucine zipper domain or a derivative thereof. In some embodiments, the trimerization domain is a T4 bacteriophage fibritin motif. In some embodiments, the trimerization domain is a eukaryotic GNC4 transcription factor motif or a derivative thereof. In some embodiments, the fibritin foldon domain is the trimerization domain from enterobacteria phage T4 or a derivative thereof. In some embodiments, the trimerization domain is encoded by a nucleotide sequence having at least 90% sequence identity to the sequence of SEQ ID NO: 1.

In some embodiments, the oligomerization domain is located between any antigens.

In one aspect, provided herein is a polypeptide encoded by any embodiment of an RNA described herein.

Given the disclosure of this application, one of skill in the art could substitute any antigen of interest into the RNA constructs described herein.

### Lipid Compositions and Methods of Preparing

Provided herein, in some embodiments, are compositions of an RNA molecule described herein and a delivery vehicle. Further provided herein are methods relating to the RNA molecule described herein and a delivery vehicle. In some embodiments, an RNA delivery vehicle is a nanoparticle (e.g., LNP).

A delivery vehicle can be non-virion particles, i.e., they are not a virion. Thus, in some embodiments, the delivery vehicle does not comprise a protein capsid. By avoiding the need to create a capsid, a delivery vehicle does not utilize a packaging cell line, thus permitting easier up-scaling for commercial production and minimizing the risk that dangerous infectious viruses will inadvertently be produced. Various materials are suitable delivery particles which can deliver RNA to a vertebrate cell in vivo. Exemplary delivery materials are (i) amphiphilic lipids which can form liposomes and (ii) non-toxic and biodegradable polymers which can form microparticles. Other delivery methods may include, but are not limited to, exosomes and cationic nano-emulsions.

Where delivery is by liposome, the RNA can be encapsulated or adsorbed; where delivery is by polymeric microparticle, the RNA can be encapsulated or adsorbed. A third delivery material is the particulate reaction product of a polymer, a crosslinker, a RNA, and a charged monomer. In certain embodiments, the delivery particle described herein comprises a liposome adsorbing RNA molecules described herein.

In some embodiments, the RNA is encapsulated within LNPs. This means that RNA inside the particles is separated from any external medium by the delivery material, and encapsulation has been found to protect RNA from RNase digestion. Encapsulation can take various forms. For example, in some embodiments, the delivery material forms an outer layer around an aqueous RNA-containing core. In some embodiments, the composition of the RNA encapsulated by the LNP is lyophilized.

In some embodiments, RNA is adsorbed to the surface of the LNPs. This means, in some embodiments, that RNA is not separated from any external medium by the delivery material, unlike the RNA genome of a natural virus.

In some embodiments, the LNP has a pH of about 5 to about 7. In some embodiments, the LNP has a pH of about 5.2 to about 6.8. In some embodiments, the LNP has a pH of about 5.4 to about 6.6. In some embodiments, the LNP has a pH of about 5.5 to about 6.5. In some embodiments, the LNP has a pH of about 5.2 to about 6.4. In some embodiments, the LNP has a pH of about 5 to about 6.2.

In some embodiments, the RNA of the present disclosure is formulated in lipid nanoparticles (LNPs) having a diameter of about 40 nanometer (nm) to about 600 nm. In some embodiments, the LNP has a diameter of about 50 to about 600 nm, about 100 to about 600 nm, about 150 to about 600 nm, about 200 to about 600 nm, about 250 to about 600 nm, or about 300 to about 600 nm. In some embodiments, the LNP has a diameter of about 50 to about 550 nm, about 50 to about 500 nm, about 50 to about 450 nm, about 50 to about 400 nm, about 50 to about 350 nm, or about 50 to about 300 nm.

In some embodiments, the LNP has a diameter no more than about 300 nm. In some embodiments, the LNP has a diameter no more than about 250 nm. In some embodiments, the LNP has a diameter no more than about 200 nm. In some embodiments, the LNP has a diameter no more than about 190 nm. In some embodiments, the LNP has a diameter no more than about 180 nm. In some embodiments, the LNP has a diameter no more than about 170 nm. In some embodiments, the LNP has a diameter no more than about 160 nm. In some embodiments, the LNP has a diameter no more than about 150 nm. In some embodiments, the LNP has a diameter no more than about 140 nm. In some embodiments, the LNP has a diameter no more than about 130 nm. In some embodiments, the LNP has a diameter no more than about 120 nm.

Further provided herein are methods of preparing a RNA-LNP composition comprising: a) mixing an ethanol phase comprising one or more lipids and an aqueous phase comprising the RNA molecules described herein; and b) purifying the RNA-LNP generated from step a).

In some embodiments, the one or more lipids comprises an ionizable lipid. In some embodiments, the one or more lipids comprises cholesterol. In some embodiments, the one or more lipids comprises a phospholipid. In some embodiments, the one or more lipids is pegylated.

In some embodiments, the LNP:RNA (N:P) ratio is about 6 to about 10. In some embodiments, the N:P ratio is about 6.5 to about 9, about 7 to about 9, about 7.5 to about 9, about 8 to about 9, about 6 to about 9.5, about 6 to about 9, about 6 to about 8.5, or about 6 to about 8.

In some embodiments, the aqueous phase comprises tris, sodium chloride, sucrose, or combinations thereof. In some embodiments, the aqueous phase comprises tris. In some embodiments, the aqueous phase comprises sodium chloride. In some embodiments, the aqueous phase comprises sucrose. In some embodiments, the aqueous phase comprises or consists of tris, sodium chloride, and sucrose.

In some embodiments, a lipid nanoparticle (LNP) formulation described herein comprises, consists essentially of, or consists of (i) a neutral phospholipid (ii) a sterol, e.g., cholesterol; (iii) a pegylated lipid optionally and (iv) a ionizable lipid with the molar ratio within ranges of neutral phospholipid: 5%-20%, sterol: 18.5%-58.5%, pegylated lipid: 1%-4%, ionizable lipid: 30%-70%, the total summation of the mole ratio of the lipids is 100%, optionally 10:40.5:1.5:48.

In some embodiments, the LNP comprises a DSPC:cholesterol:DMG-PEG 2000; and an ionizable lipid with the mole ratio within the ranges of DSPC: 5%-20%, cholesterol: 18.5%-58.5%, DMG-PEG 2000: 1%-4%, ionizable lipid: 30%-70%, and the total summation of the mole ratio of the lipids is 100%.

In some embodiments, the lipid is a cationic lipid, also called ionizable lipid. In some embodiments, useful cationic lipids generally contain a nitrogen atom that is positively charged under physiological conditions e.g., as a tertiary or quaternary amine. This nitrogen can be in the hydrophilic head group of an amphiphilic surfactant. The lipid may be selected from, but is not limited to, 1,2-dioleoyloxy-3-(trimethylammonio) propane (DOTAP), 3′-[N—(N′,N′-Dimethylaminoethane)-carbamoyl]cholesterol (DC cholesterol), dimethyldioctadecyl-ammonium (DDA e.g., the bromide), 1,2-Dimyristoyl-3-Trimethyl-AmmoniumPropane (DMTAP), dipalmitoyl(C16:0)trimethyl ammonium propane (DPTAP), distearoyltrimethylammonium propane (DSTAP). Other useful cationic lipids are: benzalkonium chloride (BAK), benzethonium chloride, cetramide (which contains tetradecyltrimethylammonium bromide and possibly small amounts of dedecyltrimethylammonium bromide and hexadecyltrimethyl ammonium bromide), cetylpyridinium chloride (CPC), cetyl trimethylammonium chloride (CTAC), N,N′,N′-polyoxyethylene (10)-N-tallow-1,3-diaminopropane, dodecyltrimethylammonium bromide, hexadecyltrimethyl-ammonium bromide, mixed alkyl-trimethyl-ammonium bromide, benzyldimethyldodecylammonium chloride, benzyldimethylhexadecyl-ammonium chloride, benzyltrimethylammonium methoxide, cetyldimethylethylammonium bromide, dimethyldioctadecyl ammonium bromide (DDAB), methylbenzethonium chloride, decamethonium chloride, methyl mixed trialkyl ammonium chloride, methyl trioctylammonium chloride), N,N-dimethyl-N-[2 (2-methyl-4-(1,1,3,3tetramethylbutyl)-phenoxy]-ethoxy)ethyl]-benzenemetha-naminium chloride (DEBDA), dialkyldimetylammonium salts, [1-(2,3-dioleyloxy)-propyl]-N,N,N,trimethylammonium chloride, 1,2-diacyl-3-(trimethylammonio) propane (acyl group=dimyristoyl, dipalmitoyl, distearoyl, dioleoyl), 1,2-diacyl-3-(dimethylammonio) propane (acyl group=dimyristoyl, dipalmitoyl, distearoyl, dioleoyl), 1,2-dioleoyl-3-(4′-trimethyl-ammonio) butanoyl-sn-glycerol, 1,2-dioleoyl 3-succinyl-sn-glycerol choline ester, cholesteryl (4′-trimethylammonio) butanoate), N-alkyl pyridinium salts (e.g., cetylpyridinium bromide and cetylpyridinium chloride), N-alkylpiperidinium salts, dicationic bolaform electrolytes (Cl2Me6; Cl2BU6), dialkylglycetylphosphorylcholine, lysolecithin, L-α dioleoylphosphatidylethanolamine, cholesterol hemisuccinate choline ester, lipopolyamines, including but not limited to dioctadecylamidoglycylspermine (DOGS), dipalmitoyl phosphatidylethanol-amidospermine (DPPES), lipopoly-L (or D)-lysine (LPLL, LPDL), poly (L (or D)-lysine conjugated to N-glutarylphosphatidylethanolamine, didodecyl glutamate ester with pendant amino group (C₁₂GluPhCₙN⁺), ditetradecyl glutamate ester with pendant amino group (Cl2GluPhCₙN^{|}), cationic derivatives of cholesterol, including but not limited to cholesteryl-3β-oxysuccinamidoethylenetrimethylammonium salt, cholesteryl-3β-oxysuccinamidoethylenedimethylamine, cholesteryl-3β-carboxyamidoethylenetrimethylammonium salt, and cholesteryl-3β-carboxyamidoethylenedimethylamine.

In some embodiments, the lipid has the following structure:

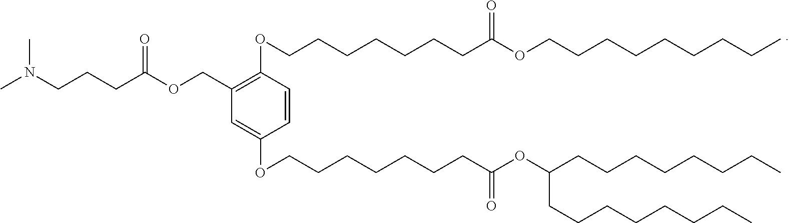 [SMILES: CCCCCCCCCOC(=O)CCCCCCCOc1ccc(OCCCCCCCC(=O)OC(CCCCCCCC)CCCCCCCC)cc1COC(=O)CCCN(C)C]

In some embodiments, the lipid has the following structure:

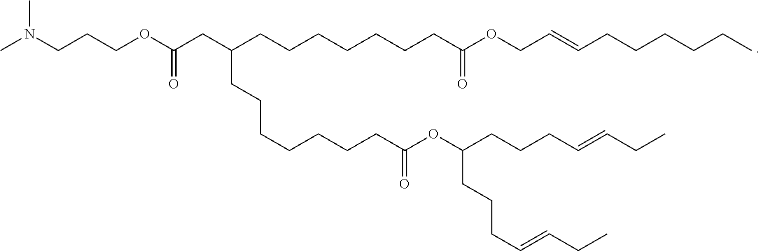 [SMILES: CC/C=C/CCCC(CCC/C=C/CC)OC(=O)CCCCCCCC(CCCCCCCC(=O)OC/C=C/CCCCCC)CC(=O)OCCCN(C)C]

In some embodiments, an RNA delivery vehicle is a nanoparticle that comprises at least one lipid. In some embodiments, the lipid may be a neutral lipid. In some embodiments, the lipid is a phospholipid. The phospholipid may be selected from, but is not limited to, DDPC, 1,2-Didecanoyl-sn-Glycero-3-phosphatidylcholine, DEPA, 1,2-Dierucoyl-sn-Glycero-3-Phosphate, DEPC, 1,2-Erucoyl-sn-Glycero-3-phosphatidylcholine, DEPE, 1,2-Dierucoyl-sn-Glycero-3-phosphatidylethanolamine, DEPG, 1,2-Dipalmitoylphosphatidylglycerol, DLOPC, 1,2-Linoleoyl-sn-Glycero-3-phosphatidylcholine, DLPA, 1,2-Dilauroyl-sn-Glycero-3-Phosphate, DLPC, 1,2-Dilauroyl-sn-Glycero-3-phosphatidylcholine, DLPE, 1,2-Dilauroyl-sn-Glycero-3-phosphatidylethanolamine, DLPG 1,2-Dilauroyl-sn-Glycero-3 (phosphorylglycerol), DLPS 1,2-Dilauroyl-sn-Glycero-3-phosphatidylserine, DMG, 1,2-Dimyristoyl-sn-glycero-3-phosphoethanolamine, DMPA, 1,2-Dimyristoyl-sn-Glycero-3-Phosphate, DMPC, 1,2-Dimyristoyl-sn-Glycero-3-phosphatidylcholine, DMPE, 1,2-Dimyristoyl-sn-Glycero-3-phosphatidylethanolamine, DMPG, 1,2-Dimyristoyl-sn-glycero-3-phosphoglycerol, DMPS, 1,2-Dimyristoyl-sn-Glycero-3-phosphatidylserine, DOPA, 1,2-Dioleoyl-sn-Glycero-3-Phosphate, DOPC, 1,2-Dioleoyl-sn-Glycero-3-phosphatidylcholine, DOPE, 1,2-Dioleoyl-sn-Glycero-3-phosphatidylethanolamine, DOPG, 1,2-Dipalmitoylphosphatidylglycerol, DOPS, 1,2-Dioleoyl-sn-Glycero-3-phosphatidylserine, DPPA, 1,2-Dipalmitoyl-sn-Glycero-3-Phosphate, DPPC, 1,2-Dipalmitoyl-sn-Glycero-3-phosphatidylcholine, DPPE, 1,2-Dipalmitoyl-sn-Glycero-3-phosphatidylethanolamine, DPPG, 1,2-Dipalmitoyl-sn-glycero-3-phosphoglycerol, DPPS, 1,2-Dipalmitoyl-sn-Glycero-3-phosphatidylserine, DPyPE, 1,2-diphytanoyl-sn-glycero-3-phosphoethanolamine, DSPA, 1,2-Distearoyl-sn-Glycero-3-Phosphate, DSPC, 1,2-Distearoyl-sn-Glycero-3-phosphatidylcholine, DSPE, 1,2-Diostearpyl-sn-Glycero-3-phosphatidylethanolamine, DSPG, 1,2-Distearoyl-sn-Glycero-3-phosphorylglycerol, DSPS, 1,2-diundecanoyl-sn-glycero-phosphocholine, DUPC, 1,2-Distearoyl-sn-Glycero-3-phosphatidylserine, EPC, Egg-PC, HEPC, Hydrogenated Egg PC, HSPC, High purity Hydrogenated Soy PC, HSPC, Hydrogenated Soy PC, Lysopc Myristic, 1-Myristoyl-sn-Glycero-3-phosphatidylcholine, LYSOPC PALMITIC, 1-Palmitoyl-sn-Glycero-3-phosphatidylcholine, LYSOPC STEARIC, 1-Stearoyl-sn-Glycero-3-phosphatidylcholine, Milk Sphingomyelin, MPPC, 1-Myristoyl,2-palmitoyl-sn-Glycero 3-phosphatidyl choline, MSPC, 1-Myristoyl,2-stearoyl-sn-Glycero-3-phosphatidylcholine, PMPC, 1-Palmitoyl,2-myristoyl-sn-Glycero-3-phosphatidylcholine, POPC, 1-Palmitoyl,2-oleoyl-sn-Glycero-3-phosphatidylcholine, POPE, 1-Palmitoyl-2-oleoyl-sn-Glycero-3-phosphatidylethanolamine, POPG, 1,2-Dioleoyl-sn-Glycero-3-Phospho-rac-(1-glycerol)], PSPC, 1-Palmitoyl,2-stearoyl-sn-Glycero-3-phosphatidylcholine, SMPC, 1-Stearoyl,2-myristoyl-sn-Glycero-3-phosphatidylcholine, SOPC, 1-Stearoyl,2-oleoyl-sn-Glycero-3-phosphatidylcholine, SPPC, 1-Stearoyl,2-palmitoyl-sn-Glycero-3-phosphatidylcholine.

In some embodiments, an RNA delivery vehicle is a nanoparticle that comprises at least one lipid. In some embodiments, the lipid may be a PEGylated lipid (PEG). In some embodiments, the PEGylated lipid comprises a polyethylene glycol moiety. In some embodiments, a PEG lipid includes but is not limited to PEG-modified phosphatidylethanolamines, PEG-modified phosphatidic acids, PEG-modified ceramides, PEG-modified dialkylamines, PEG-modified diacylglycerols, PEG-modified dialkylglycerols, or combinations thereof. In some embodiments, a PEGylated lipid is PEG-c-DOMG, PEG-DMG, PEG-DLPE, PEG-DMPE, PEG-DPPC, or a PEG-DSPE lipid. In some embodiments, the PEGylated lipid is DMG-PEG, i.e. PEG-conjugated 1,2-dimyristoyl-sn-glycero-3-phosphoethanolamine-N-[methoxy (polyethylene glycol). In some embodiments, the lipid is DMG-PEG 2000, i.e. 1,2-dimyristoyl-rac-glycero-3-methoxypolyethylene glycol-2000. In some embodiments, the pegylated lipid comprises 1,2-dimyristoyl-rac-glycero-3-methoxypolyethylene glycol-2000 (DMG-PEG 2000), DMG-PEG 3500, DMG-PEG 5000, DTDAM-PEG 2000 (ALC-0159), DTDAM-PEG 5000, DMG-C-PEG 2000, DMG-C-PEG 5000, DSG-PEG 2000, DSG-PEG 5000, DPG-PEG 2000, DPG-PEG 5000, or any combination thereof.

In some embodiments, the lipid is a structural lipid. In some embodiments, the structural lipid includes, but is not limited to, cholesterol, fecosterol, sitosterol, ergosterol, campesterol, stigmasterol, brassicasterol, tomatidine, tomatine, ursolic acid, alpha-tocopherol, or combinations thereof. In some embodiments, the structural lipid is cholesterol. In some embodiments, the structural lipid is cholesterol, a corticosteroid (such as prednisolone, dexamethasone, prednisone, and hydrocortisone), or a combination thereof.

In certain embodiments, provided herein is a pharmaceutically acceptable composition comprising the RNA-LNP described herein, and a pharmaceutically acceptable carrier.

In some embodiments, pharmaceutically acceptable salts of the lipids described herein include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, digluconate, dodecylsulfate, ethanesulfonate, formate, fumarate, glucoheptanoate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, palmoate, pectinate, persulfate, 3-phenylpropionate, phosphate, picrate, pivalate, propionate, salicylate, succinate, sulfate, tartrate, thiocyanate, tosylate, trifluoroacetate, and undecanoate.

### Methods of Treatment

Provided herein are compositions (e.g., pharmaceutical compositions), methods, kits and reagents for prevention and/or treatment of diseases or conditions in humans and other mammals.

The disclosure herein provides a method of mixing the lyophilized RNA with a liquid LNP solution to make a pharmaceutical composition. In certain embodiments, the liquid LNP solution is added to the lyophilized RNA. In certain embodiments, the lyophilized RNA and liquid LNP solution are mixed at room temperature. In certain embodiments, the lyophilized RNA and liquid LNP solution are mixed prior to clinical use. In some embodiments, the RNA adsorbed to the surface of the LNP in a liquid state is administered to the subject at least two times. In some embodiments, the RNA adsorbed to the surface of the LNP in a liquid state is administered to the subject two times, three times, or four times. In some embodiments, the RNA adsorbed to the surface of the LNP in a liquid state is administered to the subject once every two, three, or four weeks.

In some embodiments, the lyophilized RNA encapsulated in the LNP is mixed with sterile water. In some embodiments, the lyophilized RNA encapsulated in the LNP and sterile water are administered to the subject intramuscularly. In some embodiments, the lyophilized RNA encapsulated in the LNP is administered to the subject at least two times. In some embodiments, the lyophilized RNA encapsulated in the LNP is administered to the subject two times, three times, or four times. In some embodiments, the lyophilized RNA encapsulated in the LNP is administered to the subject once every two, three, or four weeks. Prophylactic protection from an antigen can be achieved following administration of a RNA vaccine or a therapeutic of the present disclosure. In certain embodiments, it is sufficient to administer the vaccine or therapeutic twice. It is possible, although less desirable, to administer the vaccine or therapeutic to an infected individual to achieve a therapeutic response.

In some embodiments, the RNA is encapsulated in an LNP in a sterile buffer. In some embodiments, the sterile buffer is water. In some embodiments, the sterile buffer comprises salts. In some embodiments, the RNA encapsulated in an LNP is administered to the subject at least two times. In some embodiments, the RNA encapsulated in an LNP is equilibrated at room temperature for about 5-20 minutes or 10-15 minutes before administration. In some embodiments, the RNA encapsulated in an LNP is administered to the subject two times, three times, or four times. In some embodiments, the RNA encapsulated in an LNP is administered to the subject once every two, three, or four weeks.

A method of eliciting an immune response in a subject against an antigen is provided in aspects of the present disclosure. The method involves administering to the subject a RNA vaccine or therapeutic comprising a RNA molecule having an open reading frame encoding at least one antigenic polypeptide and a delivery vehicle, such as an LNP, thereby inducing in the subject an immune response. An “anti-antigenic polypeptide antibody” is a serum antibody the binds specifically to the antigenic polypeptide.

A “prophylactically effective dose” as used herein is a therapeutically effective dose that prevents infection with the virus at a clinically acceptable level. In some embodiments, the therapeutically effective dose is a dose listed in a package insert for the vaccine or therapeutic. A traditional vaccine, as used herein, refers to a vaccine other than the RNA vaccine or therapeutic of the present disclosure. For instance, a traditional vaccine includes, but is not limited, to live microorganism vaccines, killed microorganism vaccines, subunit vaccines, protein antigen vaccines, DNA vaccines, VLP vaccines, etc. In exemplary embodiments, a traditional vaccine is a vaccine that has achieved regulatory approval and/or is registered by a national drug regulatory body, for example, the Food and Drug Administration (FDA) in the United States or the European Medicines Agency (EMA).

A method of treating or preventing disease in a subject comprising administering to the subject an effective amount the RNA-lipid composition described herein is provided in this disclosure.

In some embodiments, the disease is caused by a virus is selected from the group consisting of a Influenza virus, Rabies virus, respiratory syncytial virus (RSV) and coronavirus.

In some embodiments, the disease is caused by coronavirus. In some embodiments, the coronavirus is selected from the group consisting of 229E (alpha coronavirus), NL63 (alpha coronavirus), OC43 (beta coronavirus), HKU1 (beta coronavirus), MERS-CoV (MERS), SARS-CoV (SARS), and SARS-CoV-2 (COVID-19). In some embodiments, the coronavirus is SARS-CoV-2.

In some embodiments, the RNA adsorbed to the surface of the LNP is in a liquid state, and optionally at a temperature of 2-8° C.

In some embodiments, the RNA and LNP is administered to the subject intramuscularly.

In some embodiments, the lyophilized RNA encapsulated in the LNP is mixed with sterile water.

In some embodiments, the lyophilized RNA encapsulated in the LNP and sterile water are administered to the subject intramuscularly.

In some embodiments, the RNA adsorbed to the surface of the LNP in a liquid state or lyophilized RNA encapsulated in the LNP is administered to the subject at least two times.

In some embodiments, the RNA adsorbed to the surface of the LNP in a liquid state or lyophilized RNA encapsulated in the LNP is administered once every one, two, three, or four weeks.

In some embodiments, the RNA vaccine or therapeutic is administered to a subject (e.g., parenteral and transmucosal (e.g., buccal, sublingual, palatal, gingival, nasal, vaginal, rectal, or transdermal)). Parenteral administration includes, e.g., intravenous, intramuscular, intra-arterial, intradermal, subcutaneous, intraperitoneal, intraventricular, and intracranial administration. In some embodiments, the RNA vaccine or therapeutic is administered intramuscularly. In some embodiments, the RNA vaccine or therapeutic is administered intravenously. Other modes of delivery include, but are not limited to, the use of liposomal formulations, intravenous infusion, transdermal patches, etc.

In some embodiments, the vaccine or therapeutic is administered to the subject at a dose of 1 μg-100 μg, optionally 5, 30, or 50 μg. The exact amount will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

In some embodiments, the vaccine or therapeutic is administered to the subject more than once. In some embodiments, the vaccine or therapeutic is administered to the subject at least two times. In some embodiments, the second dose is administered to the subject about 8 weeks following the initial or prime dose. In some embodiments, the second dose is administered to the subject about 7 weeks following the prime dose. In some embodiments, the second dose is administered to the subject about 6 weeks following the prime dose. In some embodiments, the second dose is administered to the subject about 5 weeks following the prime dose. In some embodiments, the second dose is administered to the subject about 4 weeks following the prime dose. In some embodiments, the second dose is administered to the subject about 3 weeks following the prime dose. In some embodiments, the second dose is administered to the subject about 2 weeks following the prime dose. In some embodiments, the second dose is administered to the subject about 1 week following the prime dose.

### Kits

In some embodiments, the present disclosure also provides kits comprising: i) an RNA; ii) a delivery vehicle, such as a liquid LNP solution; iii) instructions for mixing the RNA with the delivery vehicle to prepare an immunogenic composition; and iv) instructions for administration of the immunogenic composition to stimulate an immune response against the antigen in a mammalian subject, such as a human subject in need thereof.

In some embodiments, the RNA is stored at or below about −50° C. In some embodiments, the RNA is stored at or below about −60° C. In some embodiments, the RNA is stored at or below about −70° C. In some embodiments, the RNA is stored at about −80° C. In some embodiments, the RNA-LNP vaccine is stored at or below about 10° C. In some embodiments, the RNA-LNP vaccine is stored at or below about 0° C. In some embodiments, the RNA-LNP vaccine is stored at or below about −10° C. In some embodiments, the RNA-LNP vaccine is stored at or below about −15° C. In some embodiments, the RNA-LNP vaccine is stored at or below about −20° C.

### Definitions

To facilitate an understanding of the present invention, a number of terms and phrases are defined below.

Unless defined otherwise, all technical and scientific terms used herein have the same meaning as commonly understood by one of ordinary skill in the art to which this invention belongs. The abbreviations used herein have their conventional meaning within the chemical arts.

Throughout the description, where systems are described as having, including, or comprising specific components, or where processes and methods are described as having, including, or comprising specific steps, it is contemplated that, additionally, there are systems of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of an apparatus or a method described herein can be combined in a variety of ways without departing from the spirit and scope of the present invention, whether explicit or implicit herein. For example, where reference is made to a particular component of a system, that component can be used in various embodiments of systems of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

The articles “a” and “an” are used in this disclosure to refer to one or more than one (i.e., to at least one) of the grammatical object of the article, unless the context is inappropriate. By way of example, “an element” means one element or more than one element.

The term “and/or” is used in this disclosure to mean either “and” or “or” unless indicated otherwise.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a +10%, +5%, +3% or +2% variation from the nominal value unless otherwise indicated or inferred from the context.

At various places in the present specification, variable or parameters are disclosed in groups or in ranges. It is specifically intended that the description include each and every individual sub-combination of the members of such groups and ranges. For example, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

As a general matter, formulations specifying a percentage are by weight unless otherwise specified. Further, if a variable is not accompanied by a definition, then the previous definition of the variable controls.

The term “dried RNA” or as used herein has to be understood as RNA that has been lyophilized, or spray-dried, or spray-freeze dried as defined herein to obtain a temperature stable dried RNA (powder).

“Cryoprotectants” are known in the art and include without limitation, sucrose, trehalose, and glycerol. A cryoprotectant exhibiting low toxicity in biological systems is generally used.

The terms “lyophilization” include the related terms “cryodesiccation,” “lyophilizing,” or “freeze drying,” and typically relates to a process which allows reduction of a solvent (e.g., water) content of a frozen sample (preferably a solution containing an RNA molecule and a cryoprotectant as described herein) in one or more steps via sublimation. In the context of the present disclosure, lyophilization is typically carried out by freezing a sample and subsequently drying the sample via sublimation, optionally by reducing the surrounding pressure and/or by heating the sample so that the solvent sublimes directly from the solid phase to the gas phase.

“Polynucleotide,” “nucleic acid,” or “nucleotide” are used interchangeably herein and refer to chains of nucleotides of any length, and comprise DNA and RNA. In some embodiments, the nucleotides are deoxyribonucleotides, ribonucleotides, modified nucleotides or bases, and/or their analogs, or any substrate that is incorporated into a chain by DNA or RNA polymerase. A polynucleotide may comprise modified nucleotides, such as methylated nucleotides and their analogs. If present, modification to the nucleotide structure is imparted before or after assembly of the chain. In some embodiments, the sequence of nucleotides is interrupted by non-nucleotide components. In some embodiments, a polynucleotide is further modified after polymerization, such as by conjugation with a labeling component. Other types of modifications comprise, for example, “caps,” substitution of one or more of the naturally occurring nucleotides with an analog, internucleotide modifications such as, for example, those with uncharged linkages (e.g., methyl phosphonates, phosphotriesters, phosphoamidates, carbamates) and with charged linkages (e.g., phosphorothioates, phosphorodithioates), those containing pendant moieties, such as, for example, proteins (e.g., nucleases, toxins, antibodies, signal peptides, poly-L-lysine), those with intercalators (e.g., acridine, psoralen), those containing chelators (e.g., metals, radioactive metals, boron, oxidative metals), those containing alkylators, those with modified linkages (e.g., alpha anomeric nucleic acids), as well as unmodified forms of the polynucleotide(s). In some embodiments, any of the hydroxyl groups ordinarily present in the sugars are replaced, for example, by phosphonate groups, phosphate groups, protected by standard protecting groups, or activated to prepare additional linkages to additional nucleotides, or are conjugated to solid supports. In some embodiments, the 5′ and 3′ terminal OH is phosphorylated or substituted with amines or organic capping group moieties of from 1 to 20 carbon atoms. Other hydroxyls may also be derivatized to standard protecting groups. In some embodiments, polynucleotides also contain analogous forms of ribose or deoxyribose sugars, comprising, for example, 2′-O-methyl-, 2′-O-allyl, 2′-fluoro- or 2′-azido-ribose, carbocyclic sugar analogs, alpha- or beta-anomeric sugars, epimeric sugars such as arabinose, xyloses or lyxoses, pyranose sugars, furanose sugars, sedoheptuloses, acyclic analogs and abasic nucleoside analogs such as methyl riboside. In some embodiments, one or more phosphodiester linkages are replaced by alternative linking groups. These alternative linking groups comprise, but are not limited to, embodiments wherein phosphate is replaced by P(O)S (“thioate”), P(S)S (“dithioate”), (O)NRi (“amidate”), P(O)R, P(O)OR′, CO or CH2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, comprising RNA and DNA. In a polynucleotide, when referring to a T, a T means U (Uracil) in RNA and T (Thymine) in DNA.

The term “messenger RNA” (mRNA) refers to one type of a polynucleotide that encodes a (at least one) polypeptide (a naturally-occurring, non-naturally-occurring, or modified polymer of amino acids) and can be translated to produce the encoded polypeptide in vitro, in vivo, in situ or ex vivo.

As used herein, the terms “termini” or “terminus,” when referring to polypeptides, RNA molecules, or polynucleotides, refers to an extremity of a polypeptide, RNA molecule, or polynucleotide respectively. Such extremity is not limited only to the first or final site of the polypeptide, RNA molecule, or polynucleotide but may include additional amino acids or nucleotides in the terminal regions. Polypeptide-based molecules may be characterized as having both an N-terminus (terminated by an amino acid with a free amino group (NH2)) and a C-terminus (terminated by an amino acid with a free carboxyl group (COOH)). Proteins are in some cases made up of multiple polypeptide chains brought together by disulfide bonds or by non-covalent forces (multimers, oligomers). These proteins have multiple N-termini and C-termini. Alternatively, the termini of the polypeptides may be modified such that they begin or end, as the case may be, with a non-polypeptide based moiety such as an organic conjugate.

A “5′ untranslated region” (5′ UTR) refers to a region of an RNA (e.g., mRNA or other coding RNA) that is directly upstream (i.e., 5′) from the start codon (i.e., the first codon of an RNA, such as mRNA or other coding RNA, transcript translated by a ribosome) that does not encode a polypeptide.

A “3′ untranslated region” (3′ UTR) refers to a region of an RNA (e.g., mRNA or other coding RNA) that is directly downstream (i.e., 3′) from the stop codon (i.e., the codon of an RNA, such as mRNA or other coding RNA, transcript that signals a termination of translation) that does not encode a polypeptide.

An “open reading frame” is a continuous stretch of DNA or RNA beginning with a start codon (e.g., methionine (ATG or AUG)), and ending with a stop codon (e.g., TAA, TAG or TGA, or UAA, UAG or UGA) and typically encodes a polypeptide (e.g., protein). It will be understood that the sequences may further comprise additional elements, e.g., 5′ and 3′ UTRs, but that those elements, unlike the ORF, need not necessarily be present in a vaccine or a therapeutic of the present disclosure.

A “polyA tail” is a region of RNA (e.g., mRNA or other coding RNA) that is downstream, e.g., directly downstream (i.e., 3′), from the 3′ UTR that contains multiple, consecutive adenosine monophosphates. A polyA tail may contain 10 to 300 adenosine monophosphates (SEQ ID NO: 40). For example, a polyA tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a polyA tail contains 50 to 250 adenosine monophosphates. In a relevant biological setting (e.g., in cells, in vivo) the poly(A) sitail functions to protect RNA from enzymatic degradation, e.g., in the cytoplasm, and aids in transcription termination, and/or export of the RNA from the nucleus and translation.

The term “dose” as used herein in reference to an immunogenic composition refers to a measured portion of the immunogenic composition taken by (administered to or received by) a subject at any one time.

The term “immunization” refers to a process that increases a mammalian subject's reaction to an antigen and therefore improves its ability to resist or overcome infection and/or resist disease.

The term “vaccination” as used herein refers to the introduction of a vaccine into a body of a subject, preferably, a mammalian subject such as a human.

The term “prophylactically effective dose” includes the related terms “effective dose” and “therapeutically effective dose,” and as used herein refers to a dose that prevents infection with the virus at a clinically acceptable level.

The term “alkyl” refers to a radical of a straight-chain or branched saturated hydrocarbon group having from 1 to 32 carbon atoms (“C₁-C₃₂ alkyl”). In some embodiments, an alkyl group has 1 to 12 carbon atoms (“C₁-C₁₂ alkyl”). In some embodiments, an alkyl group has 1 to 10 carbon atoms (“C₁-C₁₀ alkyl”). In some embodiments, an alkyl group has 1 to 9 carbon atoms (“C₁-C₉ alkyl”). In some embodiments, an alkyl group has 1 to 7 carbon atoms (“C₁-C₇ alkyl”). In some embodiments, an alkyl group has 1 to 5 carbon atoms (“C₁-C₅alkyl”). In some embodiments, an alkyl group has 1 to 4 carbon atoms (“C₁-C₄ alkyl”). In some embodiments, an alkyl group has 1 to 3 carbon atoms (“C₁-C₃ alkyl”). In some embodiments, an alkyl group has 1 to 2 carbon atoms (“C₁-C₂ alkyl”). In some embodiments, an alkyl group has 1 carbon atom (“C₁ alkyl”). In some embodiments, an alkyl group has 2 to 6 carbon atoms (“C₂-C₆ alkyl”). In some embodiments, an alkyl group has 1 to 30 carbon atoms (“C₁-C₃₀ alkyl”). In some embodiments, an alkyl group has 1 to 22 carbon atoms (“C₁-C₂₂ alkyl”). In some embodiments, an alkyl group has 5 to 10 carbon atoms (“C₅-C₁₀ alkyl”). In some embodiments, an alkyl group has 7 to 17 carbon atoms (“C₇-C₁₇ alkyl”). In some embodiments, an alkyl group has 10 to 32 carbon atoms (“C₁₀-C₃₂ alkyl”).

The term “alkenyl” refers to a radical of a straight-chain or branched hydrocarbon group having from 2 to 20 carbon atoms, one or more carbon-carbon double bonds, and no triple bonds (“C₂-C₂₀ alkenyl”). In some embodiments, an alkenyl group has 2 to 10 carbon atoms (“C₂-C₁₀ alkenyl”). In some embodiments, an alkenyl group has 2 to 8 carbon atoms (“C₂-C₈ alkenyl”). In some embodiments, an alkenyl group has 2 to 6 carbon atoms (“C₂-C₆ alkenyl”). In some embodiments, an alkenyl group has 2 to 5 carbon atoms (“C₂-C₅ alkenyl”).

The term “suitable protective agent” includes the related terms “cryoprotectant” and “protective agent”, and does not cause or enhance degradation of the RNA.

### EXAMPLES

### Example 1. Synthesis of a RNA Molecule for Various Antigens

This Example describes the synthesis of a purified RNA molecule (e.g., mRNA) described herein.

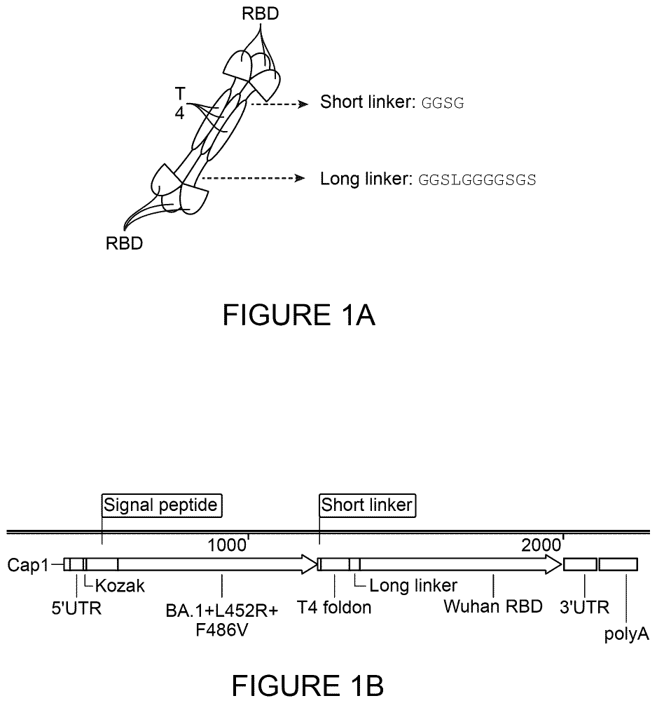

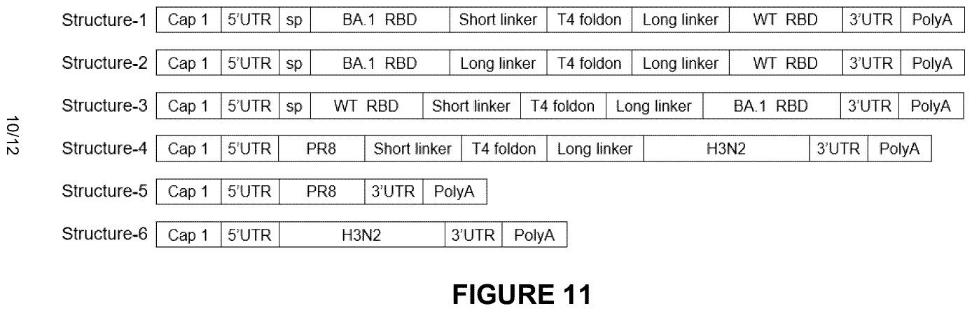

The synthesis of mRNA constructs described herein is achieved by three steps. First, the plasmid DNA encoding the antigens was amplified, extracted, and then purified. Next, the plasmid was linearized and purified by chromatographic purification and ethanol precipitation. Finally, using the linearized plasmid DNA as a template, the mRNA was enzymatically synthesized in vitro and stored at −80° C. after cleansing. The schematic diagram of an exemplary mRNA structures are shown in FIG. 1B and FIG. 11.

### Example 2. Preparation of an mRNA-LNP Vaccine

This example describes creating a composition of an RNA molecule and a delivery vehicle (e.g. a LNP).

The purified RNA molecule is formulated into lipid nanoparticles (LNP), which is prepared by rapid mixing of ethanol phase and aqueous phase using a microfluidic device. The aqueous phase comprises a 50 mM citrate buffer (pH 6.0) and a certain amount of mRNA, around 50-150 μg/mL. The ethanol phase comprises an ionizable lipid (Immorna), cholesterol, 1,2-Diastearoyl-sn-glycerol-3-phosphocholine (DSPC, and 1,2-dimyristoyl-rac-glycero-3-methoxy polyethylene glycol-2000 (DMG-PEG2000). These four lipid components were mixed at a molar ratio of 40:48:10:2.0 (ionizable lipid:cholesterol:DSPC:DMG-PEG2000). The generated LNP were purified in citrate buffer (pH 6.0) and the particle size, polydispersity index (PDI), and mRNA concentration were characterized. Finally, the pH was adjusted to 7.2. The mRNA-LNP were then diluted to the target concentration to obtain the mRNA-LNP vaccine product and stored at ≤−20° C. or ≤−60° C.

In this disclosure, the mRNA-LNP containing 30 μg/mL mRNA are prepared using the same method described in this section. Particle size and polydispersity index (PDI) of the mRNA-LNP were determined by Dynamic Light Scattering (DLS), and the mRNA purity was determined by agarose gel electrophoresis. The vaccine was used after equilibrating for 15 minutes at room temperature.

### Example 3. In Vitro Transfection of mRNA Vaccines

This Example describes the process taken to measure transfection efficiency of an RNA molecule or composition and ability of the mRNA vaccine to express the target protein.

To assess the transfection efficiency of mRNA vaccines in vitro, 3×10⁵ BHK-21 cells per well were seeded in 6-well plates. 2 μg of LNP-encapsulated mRNA vaccine was transfected into the BHK-21 cells. The expression of the RBD protein of SARS-CoV-2 in cells was detected using western blot. Briefly, at 24 hours post-transfection, cells transfected with mRNA vaccines were lysed by NP-40 Lysis buffer (50 mM Tris pH 7.4, 150 mM NaCl, 1% NP40, sodium pyrophosphate, β-glycerophosphate, sodium orthovanadate, sodium fluoride, EDTA, leupeptin). The mixtures were centrifuged at 13000 rpm for 5 minutes at 4° C. The supernatants were collected and boiled with SDS for 12 minutes at 95° C., separated in a 6% SDS-PAGE gel, and transferred to nitrocellulose filter membranes. After being blocked by 5% BSA, the membranes were first blotted with primary antibodies (1:1000) (SARS-CoV-2 (2019-nCoV) Spike Rabbit PAb) and then incubated with horseradish peroxidase (HRP) conjugated secondary antibodies (1:10000) (IgG(H+L) (HRP-labeled Goat Anti-Rabbit IgG(H+L))) and visualized with Chemiluminescent Reagent (Chemiluminescent HRP Substrate).

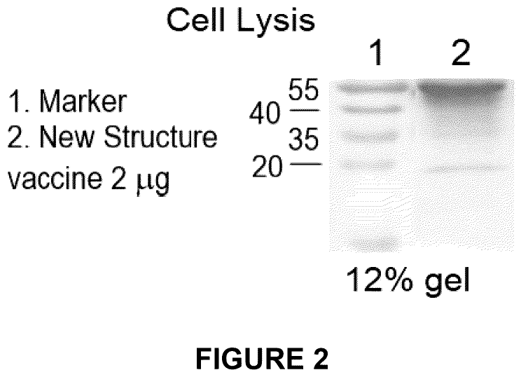

Hours post-transfection, Western blot was used to detect the target RBD protein in cell lysis. Based on the expression result of the target RBD protein shown in FIG. 2, it can be concluded that the mRNA-LNP vaccine can express the target protein.

### Example 4. In Vivo Evaluation of mRNA-LNP Vaccines

This Example describes the time line of testing an mRNA-LNP composition on mice.

Animal studies were carried out at the Yangtze Delta Region Research Institute of Tsinghua University (Zhejiang). BALB/c mice (6-8 weeks of age) were divided into groups of n=4 or n=5. On day 0 (prime injection) and day 14 or 21 (boost), three groups of mice were immunized intramuscularly with the mRNA-LNP vaccine (2 μg), and buffer vehicle, respectively. Serum was collected before the first vaccination and on days 14, 21, and 28. All collected samples were cryopreserved following standard protocols.

### Example 5. Quantification of Antibody Binding Titers Against SARS-CoV-2 Spike Protein by ELISA Assay

This Examples describes the process of measuring antibody titers from a sample.

Antibody binding titers against SARS-CoV-2 spike protein (RBD, Histag) were quantified by enzyme-linked immunosorbent assay (ELISA). Briefly, 0.5 μg/ml SARS-CoV-2 spike protein (RBD, His tag) diluted in carbonate-bicarbonate buffer was pre-coated onto 96-well transparent polystyrene microplate overnight at 4° C. After washing with PBS-T (0.05% Tween-20 in PBS) three times, the coated plates were blocked with 300 μL blocking buffer (15% normal goat serum and 2% bovine serum albumin in PBS-T) for 1 h at 37° C. Serum samples were 2-fold serially diluted in blocking buffer, transferred to the plates, and incubated for 1 h at 37° C. After washing, plates were incubated with HRP-conjugated rabbit anti-mouse IgG H+L for 1 h at 37° C. Plates were washed and incubated with TMB Single-Component Substrate solution for 7 min at 37° C., and the reaction was stopped with ELISA stop solution. Absorbance was read at 450 nm on a microplate reader, and ELISA titers were determined using non-linear 4-parameter variable slope analysis in GraphPad Prism 8 software.

### Example 6. Quantification of SARS-CoV-2 Neutralizing Antibody by a Pseudotyped Virus-Based Assay

This Example describes the process to measure the neutralizing antibody titer from a sample.

In brief, the serum samples to be tested were inactivated in the water bath at 56° C. for 30 min to inactivate the complement system found in the innate immune system. Then the serum samples were diluted 20-fold with DMEM medium and filtered with a 0.22 μm filter. Filtered serum sample was added to a 96-well plate and serially cut at 3-fold until a 4860-fold dilution was reached. The pseudovirus was diluted to 1.3×10⁴ TCID 50/mL in DMEM complete medium after rapid melting in a room temperature water bath. 50 μL of the diluted virus was added into the experiment wells. The 96-well plates were placed in an incubator at 37° C. with 5% CO₂ for 1 h. The Vero cells were lysed 5×10⁴ cells were added per well, and the samples were cultured at 37° C. in a 5% CO₂ incubator for 24 h. After 24 h, the 96-well plate was removed and equilibrated to room temperature for 30 min, 150 μL supernatant was removed from the wells, and 100 μL/well bio-Lite luciferase detection reagent was added, and the plate was shaken for 2 min. The luminescence value (RLU) was detected by a microplate reader immediately. The cell control (CC) was prepared following the same procedure without adding the pseudovirus, and the virus control (VC) was prepared following the same process without adding a diluted serum sample. The neutralizing antibody titer is expressed as the reciprocal of serum dilution at a 50% inhibition rate or the antibody concentration at a 50% inhibition rate.

The formula for calculating inhibition rate is the following:

$$Inhibition rate \% = ( 1 - \frac{Sample mean RLU - CC mean RLU}{VC mean RLU - CC mean RLU} ) \times 100 \%$$

### Example 7. Preparation and Characterization of mRNA-LNP Vaccines

This Example describes certain characteristics of a mRNA-LNP composition.

After thawing, the appearance of this mRNA-LNP vaccine is a slightly milky white dispersion, which is the expected appearance of a usual mRNA-LNP product.

In addition to appearance, the key drug characteristics of LNP-mRNA vaccines are shown in Table 1.

TABLE 1
Representative pharmaceutical characteristics of mRNA-LNP

| Formulation | Particle | Polydispersity | mRNA | Concentration |
|---|---|---|---|---|
| description | size (nm) | index | purity | (μg/mL) |
| mRNA-LNP | 124 | 0.104 | 93% | 34.1 |

### Example 8. Efficacy of mRNA-LNP Vaccines in Mice

This Example describes a study measuring antibody titers in mice.

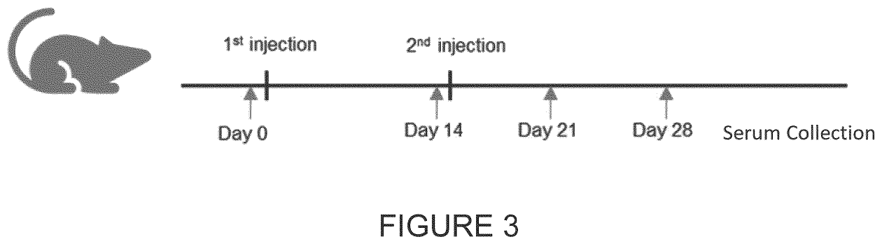

In vivo studies were carried out on the antibody response in BALB/c mice upon vaccination with mRNA-LNP Vaccine, which encodes the RBD protein of SARS-CoV-2. Mice received a prime injection and a boost after two weeks (FIG. 3).

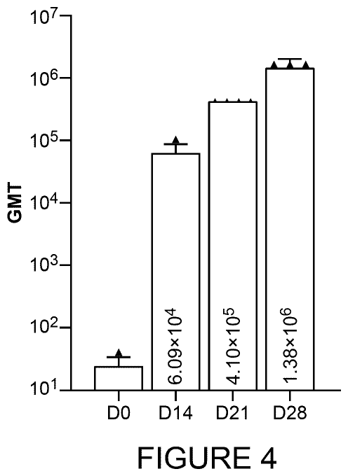

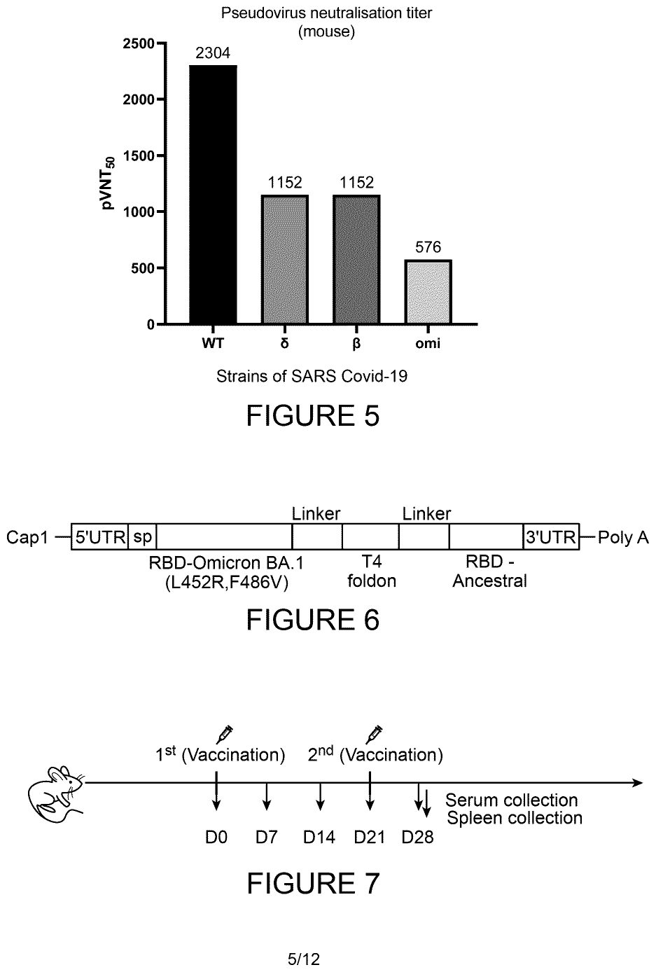

Antibody titer is highly correlated with the protective effect and durability brought by the vaccine. Therefore, it is used as the efficacy read-out in this study and presented in the form of a geometric mean titer (GMT). FIG. 4 shows that antibody titers increased substantially after two immunizations. The SARS-CoV-2 neutralizing antibody for cross-protection with different strains is shown in FIG. 5.

### Example 9. mRNA-LNP Vaccine Production

This Example describes the process of producing a mRNA-LNP vaccine.

The mRNA-LNP vaccine described in this Example has a molecular weight of 586,246 Da and is composed of Cap 1, 5′ Untranslated Region (5′ UTR), gene of interest (GOI) encoding a polypeptide containing a signal peptide (SP), the SARS-CoV-2 ancestral strain RBD, an T4 foldon domain, the RBD of Omicron BA.1 but with L452R and F486V mutations, 3′ Untranslated Region (3′ UTR), and Poly A tail. A schematic of the mRNA is provided in FIG. 6.

The final mRNA recombinant plasmid pT7 6.1 Omicron Construct C (L452R, F486V) was composed of T7 RNA polymerase promotor, 5′ Untranslated Region (5′ UTR), Kozak sequence, multiple restriction enzyme cleavage sites, gene of interest (GOI), 3′ Untranslated Region (3′ UTR), poly A, ori (plasmid replication origin), kanamycin resistance gene (KanR) and ampicillin resistance gene (AmpR) promoter. The GOI contained sequences that encoded SARS-CoV-2 ancestral strain Spike protein RBD polypeptide and Omicron BA. 1 strain Spike protein RBD polypeptide with two amino acid variants (L452R and F486V). Plasmid sequence integrity was verified by sequencing.

The recombinant plasmid pT7 6.1 Omicron Construct C (L452R, F486V) was transformed into stable competent bacteria and then inoculated on a Luria Bertani (LB) plate containing kanamycin. A single clone was selected and then inoculated into LB liquid medium and incubated overnight. After incubation, the plasmid was extracted and verified to contain the correct gene sequence by sequencing. The bacteria solution was aliquoted and stored in 10-20% glycerol and served as seed strain.

The master cell bank (MCB) was generated by inoculating one tube of the seed strain into a LB liquid medium containing 50 μg/mL kanamycin. The incubation volume was 200 μL. The medium was incubated for 8-14 hours at 30° C. and harvested as the first passage. 10-20% glycerol was added. The liquid was aliquoted into 1 mL per tube and then stored frozen at equal to or below −60° C. to serve as the master cell bank (MCB).

The working cell bank (WCB) was generated from the MCB following the same procedure as the MCB preparation and stored at equal to or below −60° C.

The manufacturing process of the mRNA Drug Substance (DS) was divided into three main units of operations: 1) manufacture of the plasmid DNA, 2) in vitro transcription of the DNA template, and 3) mRNA purification.

First, one tube of WCB was selected and inoculated into LB medium to amplify the plasmid. Cells were harvested after overnight incubation (12-20 hours at 37±1° C.).

Supercoiled plasmid DNA was isolated using P1, P2, and P3 solution. The cell lysate was filtrated and then loaded onto column packed with 6FF resin, and the first eluted fraction was collected. The supercoiled plasmid DNA was then extracted and purified using affinity chromatography and anion-exchange chromatography. Finally, the purified plasmid DNA was recovered from the anion-exchange chromatography elution fraction by adding ethanol anhydrous. The precipitated plasmid DNA was collected by centrifugation and resuspended in water for injection (WFI).

DNA plasmid linearization was performed by incubation with restriction endonuclease (BspQI). The linearized DNA was then purified by anion-exchange chromatography and ethanol precipitation. The mRNA DS was produced by in vitro enzymatic transcription using the linearized DNA plasmid as template, followed by enzymatic capping reaction and downstream purification. The process used fully linearized plasmid DNA as a template to guide T7 RNA polymerase for the in vitro transcription and synthesis of mRNA through the T7 promoter. After transcription, the template DNA was enzymatically degraded by Turbo DNase, after which the mRNA was buffer exchanged into WFI by ultrafiltration and diafiltration (UF/DF). The enzymatic capping reaction (CAP) involved using the Vaccinia virus capping enzyme, which added the 7-methylguanylate cap structure (Cap 0) to the 5′ end of the transcribed mRNA, and Cap 2′-O Methyltransferase, which converted Cap 0 to Cap 1. The Cap 1 structure stabilizes the mRNA and allows efficient translation of the mRNA in vivo.

The post-transcriptional capped mRNA was then subjected to the following steps of downstream processing: 1) Affinity chromatography purification; 2) UF/DF; 3) Cellulose chromatography purification; 4) UF/DF; 5) 0.22 μm sterile filtration, to obtain the mRNA bulk drug substance.

The final mRNA drug substance was in 18 mM citric buffer with 7% (w/v) sucrose, pH 5.8. The mRNA drug substance was stored in a sealed PETG container at or below about −60° C. The shelf life is about 3 months.

The mRNA DS control strategy included general appearance, pH, and Poly A length distribution, identity (i.e. mRNA identity, sequence of target gene), mRNA content, in vitro potency, purity (i.e. mRNA purity, capping efficiency), impurities (i.e. residual kanamycin, residual proteins, residual DNA template, residual double stranded RNA), and safety (i.e. sterility and endotoxin) tests.

The mRNA drug product (DP) was composed of a lyophilized mRNA DP (Vial 1) and an LNP dispersion (Vial 2). The LNP dispersion was synthesized using a microfluidic process, while the mRNA DS was lyophilized to a dry state to form a lyophilized mRNA DP. After reconstitution of the lyophilized mRNA by the LNP dispersion, the mRNA was preservative-free, sterile, and a white to off-white suspension at a concentration of 50 μg/mL for intramuscular injection. The reconstituted vaccine contains 19 mmol/L citric acid, and 6.5% (w/v) sucrose, pH 5.3-6.3.

Manufacture of mRNA GMP batch DP occurred. Throughout the manufacturing process, in-process control tests were implemented, and process parameters were monitored and recorded. The typical size of a GMP batch for the mRNA DP was 500 to 1,200 mL (before lyophilization). The mRNA-LNP dispersion manufacturing batch size was 1,250 to 3,000 mL. Before use, the lyophilized mRNA and LNP dispersion were equilibrated to room temperature (RT) for approximately 15 minutes. The LNP dispersion was then gently shaken for 5-10 seconds. 1.0 mL of LNP dispersion was extracted by a needle-syringe combination and added to the lyophilized cake of mRNA. After addition, the vial was inverted repeatedly for approximately 30 seconds to mix. After mixing, the reconstituted vaccine appeared as a white to off-white suspension. The vaccine should be administered within 1 hour after reconstitution.

The LNP was composed of 4 lipid components at the predefined molar ratios: 2-dimyristoyl-rac-glycero-3-methoxypolyethylene glycol-2000 (DMG-PEG2000) 1.5%, cholesterol 40.5%, 1,2-distearoyl-sn-glycero-3-phosphocholine (DSPC) 10%, and ionizable cationic lipid.

The container closure system of Vial 1 was a 2 mL borosilicate glass vial-chlorinated butyl rubber stopper for lyophilization-aluminum-plastic cap for antibiotic vials with a nominal filling volume of 0.5 mL/vial. The container closure system of Vial 2 was a 2 mL borosilicate glass vial-halogenated butyl rubber stopper for injection (brominated)-aluminum-plastic caps for antibiotic vials with a nominal filling volume of 1.0 mL/vial. The storage condition was set at 2-8° C., protected from direct sunlight.

The manufacture of mRNA DP included two segments: 1) manufacture of mRNA DP and 2) manufacture of LNP dispersion.

The mRNA DP manufacturing batch size was 500 to 1,200 mL based on the yield of the mRNA DS manufacturing process. mRNA bulk DS was first diluted with the formulation buffer (18 mmol/L citric acid, 7.0% (w/v) sucrose, pH 5.8) to a concentration of approximately 100 μg/mL. After sterile filtration, the mRNA solution was filled into 2 mL borosilicate glass vials with a nominal filling volume of 0.5 mL/vial. Lyophilization was then performed to produce the mRNA DP.

The mRNA-LNP dispersion manufacturing batch size was 1,250 to 3,000 mL. The rapid mixing of aqueous buffer with lipids dissolved in ethanol by a microfluidic mixer induced the lipid self-assembling into nanoparticles. The produced LNP was then concentrated and buffer-exchanged into citrate buffer (10 mmol/L citric acid, pH 5.5). Sucrose stock solution was added to a final concentration of 3% sucrose (w/v). After lipid concentration adjustment and subsequent sterile filtration, the LNP was filled into 2 mL borosilicate glass vials with a nominal filling volume of 1 mL/vial.

Asepsis of the lyophilization and fill/finish process was verified by mock medium filling. Throughout the manufacturing, process parameters were monitored and recorded. Process control was implemented by in-process tests.

The mRNA DP control strategy included general appearance, visible particles, pH, osmolarity, uniformity of dosage units, moisture content, particle size, polydispersity index, identity, mRNA content, % complexation efficiency, in vitro potency, purity (i.e. mRNA purity and lipid purity), impurities (i.e. residual ethanol), and safety (e.g. endotoxin and sterility) tests. Among these, uniformity of dosage units and moisture content were tested on lyophilized mRNA cake; appearance was tested for both lyophilized mRNA cake and after the reconstitution of the lyophilized mRNA with mRNA-LNP dispersion; the rest of the quality attributes were tested after reconstitution. The mRNA-LNP dispersion control strategy included general appearance, visible particles, pH, extractable volume, particle size, polydispersity index, zeta potential, lipid identities, lipid contents and molar ratio, and safety (e.g. endotoxin and sterility) tests.

The mRNA DP and LNP dispersion were stored at 2-8° C. in glass vials and protected from direct sunlight. The projected shelf life was 18 months.

### Example 10. In Vivo Immunogenicity Evaluation of mRNA-LNP Vaccine in Mice

This Example evaluates the in vivo effects of a mRNA vaccine described in Example 10 that encodes the receptor binding domain (RBD) sequences of the SARS-CoV-2 ancestral strain and variant strain (Omicron BA.1) Spike proteins. The encoded RBD sequence of Omicron variant BA.1 contains 2 Omicron BA.4/.5 strain-specific amino acid substitutes L452R and F486V. The objective of this Example is to evaluate the immunogenicity of the mRNA-LNP vaccine in mice. The immunogenicity of the vaccine was evaluated by measuring antigen-specific binding antibody titers by ELISA, pseudovirus neutralizing antibody titers, and the cellular immune response after vaccination.

### Experimental Methods

### Animal Experiment

The immunization scheme and sampling/testing plans are listed in Table 2 and Table 3.

TABLE 2
Dosing groups and immunization scheme

| | Group | |
|---|---|---|
| Treatment | Day 0 | Day 21 |

| | |
|---|---|
| Control | Dose: 0.9% NaCl; 4 female BALB/c mice; Dosing route: i.m. |
| Low dose | Dose: mRNA-LNP vaccine 4 μg; 6 female mice; |
| | Dosing route: i.m. |
| High dose | Dose: mRNA-LNP vaccine 8 μg; 6 female mice; |
| | Dosing route: i.m. |

TABLE 3
Sampling and testing plans.

| | Time | | | | |
|---|---|---|---|---|---|
| | Day | Day | Day | Day | Day |
| Samples/testing | 0 | 7 | 14 | 21 | 28 |
| Serum collection for antigen-specific | Yes | Yes | Yes | Yes | Yes |
| binding antibody measurement | | | | | |
| Serum collection for pseudovirus | — | — | — | — | Yes |
| neutralizing antibody measurement | | | | | |
| Splenocytes intracellular cytokine | — | — | — | — | Yes |
| staining and flow cytometry | | | | | |

### Test Article Preparation for Administration:

Before administration, the mRNA component and LNP dispersion were equilibrated to room temperature for about 15 minutes. The LNP dispersion was gently shaken for 5~10 seconds. 0.6 mL LNP dispersion was drawn by a needle syringe and added into the lyophilized cake of the mRNA. After addition, the vial was inverted upside down for approximately 30 seconds for thorough mixing. The reconstituted vaccine appeared as a white to off-white suspension.

### Immunization of the Mice:

A schematic of the immunization is seen in FIG. 7. Briefly, sixteen female mice of SPF grade were randomly assigned into three groups, including a control group (4 mice), a low dose group (6 mice), and a high dose group (6 mice per group). The mice were administered twice with an interval of 21 days with the date of the first administration was defined as the first day of the experiment (DO). The administration was performed by intramuscular injection (i.m.) on the right calf hindlimbs of the mice. The administration volume of the negative control group and the low-dose group was 0.08 mL/animal; the administration volume of the high-dose group was 0.16 mL/animal. The calf hindlimb was injected intramuscularly at multiple points with each point not exceeding 0.1 mL. Blood samples (50-100 μL per mouse) were drawn and spleens were harvested at the time points shown in FIG. 7.

The animals were checked daily for appearance and signs, behavioral activities, animal posture, diet, fur, irritative reaction, glandular secretions, excretions, respiratory status, and deaths. The mice were also weighed regularly.

### Serum Preparation:

Blood samples were collected into Eppendorf tubes and maintained on ice. After centrifugation at 1,500 g for 10 min at 4° C., the supernatant was immediately transferred to new tubes and stored at temperature below −70° C.

### Splenocytes Isolation:

The spleens were isolated from the mice, placed in PBS, and gently homogenized. Then the suspension was passed through a 70 μm cell strainer. Red blood cells were lysed using red blood cell lysate buffer following the manufacturer's instructions. For immediate use, after washing twice with PBS, the cells were suspended in RPMI-1640 complete medium containing 10% fetal bovine serum, 100 U/mL penicillin, and 100 μg/mL streptomycin. To save the cells for later use, a cell-freezing medium containing 90% FBS (9 mL) and 10% DMSO (1 mL) was first prepared and kept at 4° C. Then cells were taken out from the centrifuged tube and resuspended with freezing medium by pipetting up and down 20 times to break cell-cell aggregation. The cell density was adjusted to 2×10⁶/mL, after that, the cell suspension was sub-packed with cryotubes and transferred to-70° C. in the Cryobox overnight. Finally, the tubes were transferred to a liquid nitrogen tank.

### Measurement of Antigen-Specific Antibody Titers:

Binding antibody titers against SARS-CoV-2 Spike protein RBD-Omicron strain (BA.1) His-tagged (RBD-O) or SARS-CoV-2 Spike protein RBD-ancestral strain His-tagged (RBD-WT) were quantified by enzyme-linked immunosorbent assay (ELISA). Briefly, 100 μL of 1 μg/mL RBD-O or 100 μL of 0.5 μg/mL RBD-WT diluted in a coating buffer (0.05 M carbonate buffer) was pre-coated onto 96-well transparent polystyrene microplate overnight at 4° C. After washing with PBS-T (0.05% Tween-20 in PBS) three times, the coated plates were blocked with 300 μL blocking buffer (15% normal goat serum and 2% bovine serum albumin in PBS-T) for 1 h at 37° C. Serum samples were 2-fold serially diluted in blocking buffer, transferred to the plates, and incubated for 1 h at 37° C. After washing, plates were incubated with HRP-conjugated rabbit anti-mouse IgG H+L for 1 h at 37° C. Plates were washed and incubated with TMB Single-Component Substrate solution for 7 min at 37° C., and the reaction was stopped with ELISA stop solution. Absorbance was read at 450 nm on a microplate reader, and ELISA titers were determined on 2.1-fold the value of the blank and analyzed using the GraphPad Prism 8 software.

### Measurement of Neutralizing Antibody Titers:

Briefly, serum samples to be tested were inactivated in the water bath at 56° C. for 30 min to inactivate the complement system. After that, the serum samples were diluted 20 times with DMEM medium and filtered with a 0.22 m filter. The filtered serum sample was added to 96-well plate and serially diluted in 3-fold ladder until desired concentration. The pseudovirus of different SARS-CoV-2 variants of concern was diluted to 1.3×10⁴ TCID50/mL in DMEM complete medium after rapid melting in a room temperature water bath. Then 50 μL of the diluted virus was added to the experiment wells. The 96-well plates were placed in an incubator at 37° C. with 5% CO₂ for 1 h. Vero cells were digested and added into a well with 5×10⁴ cells/well, then cultured at 37° C. in a 5% CO₂ incubator for 24 h. After 24 h, the 96-well plate was equilibrated to room temperature for 30 min, 150 μL supernatant was removed from the wells, 100 μL/well bio-Lite luciferase detection reagent was added, and the plate was shaken for 2 min. The luminescence value (RLU) was detected by a microplate reader immediately. For the cell control (CC), the same procedure was repeated without adding the pseudovirus, as well as for the Virus control (VC) without adding a diluted serum sample. The neutralizing antibody titer was expressed as the reciprocal of serum dilution at a 50% inhibition rate or the antibody concentration at a 50% inhibition rate. Mathematical equation for inhibition rate calculating:

$$Inhibition rate \% = ( 1 - \frac{Sample mean RLU - CC mean RLU}{VC mean RLU - CC mean RLU} ) \times 100 \%$$

### Intracellular Cytokine Staining and Flow Cytometry

### Cell Preparation and Peptide Stimulation:

The cells were resuspended in RPMI 1640 medium containing 10% FBS and 1% PS and then 2×10⁶/200 μL cells were added to a 96 well plates, after which 25 μL peptide solution (80 μg/mL dissolved in DMSO) was added to each well. Lastly, 0.4 μL protein transport inhibitor cocktail (500× stock) was added and incubated overnight with 5% CO₂ at 37° C.

### Surface Marker Staining:

The cell suspension was centrifugated at 500 g for 5 min. The pellet was resuspended in 200 μL FACS Buffer (PBS with 0.5% BSA), the suspension was re-centrifuged at 500 g and 4° C. for another 5 min, and this step was re-conducted. Antibodies, including anti-CD3, anti-CD44, and anti-CD8, were diluted in 200 μL FACS buffer by 200-fold and used to resuspend the cells, followed by incubation on ice located in dark conditions for 30 min. Next, the cells were centrifuged at 500 g and 4° C. for 5 min. Then, the pellet was resuspended in 200 μL FACS buffer and recentrifuged in the same conditions, and this step was repeated. Finally, the cells were resuspended in 500 μL Foxp3 Fixation/Permeabilization (⅓ dilution) working solution and allowed to incubate for 20 minutes at 4° C. in the dark.

### Intracellular Cytokine Staining:

Cell suspension was centrifugated at 600 g for 5 min. The pellet was resuspended in 1 mL of 1× permeabilization buffer and then recentrifuged. This step was then repeated twice. Antibodies, including anti-IFN-γ, were diluted in 200 μL of 1× permeabilization buffer by 200 folds and used to resuspend the cells, followed by incubation for 1 hr on ice in the dark. After incubation, the cells were centrifuged at 600 g and 4° C. for 5 min. The pellet was then resuspended in 1 mL FACS buffer and recentrifuged. This step was repeated once. Finally, the stained cells were resuspended in 220 μL of FACS buffer and ready for analysis on a flow cytometer.

### Statistical Analysis:

All data are presented as mean (SD) or means±SEM. Differences between the mean values from the two groups were assessed using two-tailed Student's t-tests. Differences in mean values among more than two groups were determined using ANOVA. P<0.05 (*), P<0.01 (**) and P<0.001 (***) indicated statistically significant differences. When the sample size was ≤2, individual data were listed and no statistical analysis was performed.

### Results

### General Conditions

Generally, no obvious abnormality was observed in the appearance and signs, body weight, behavioral activities, animal posture, diet, fur, irritation reaction, glandular secretions, and respiratory status of all animals during the study period.

### Antibody Binding Titers Against SARS-CoV-2 Antigen:

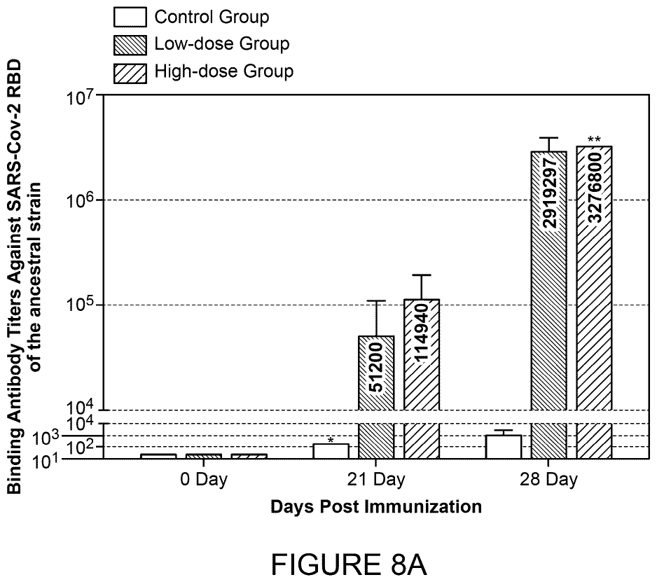

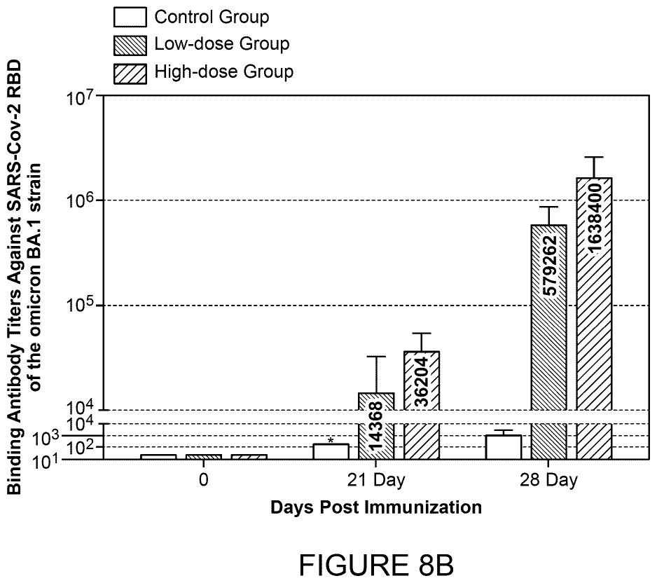

To evaluate the humoral immunogenicity of the mRNA-LNP vaccine, the antigen-specific binding antibody titers against SARS-CoV-2 RBD polypeptide were measured by ELISA in serum samples collected from mice immunized with the mRNA-LNP vaccine. As shown in FIG. 8A and FIG. 8B, the mRNA-LNP vaccine was able to stimulate high titers of antigen-specific binding antibodies against RBD polypeptides of the SARS-CoV-2 ancestral strain and Omicron BA.1 strain after just one injection. After 21 days, in the low-dose group, the mean titers of binding antibody against RBD of the original strain and the Omicron BA.1 strain reached 51,200 and 14,368, respectively. The high-dose group displayed higher titers (at 114, 940 and 36,204, respectively) against both strains than the low-dose group, showing a clear dose dependent immune effect. After boost immunization by the second injection, the antibody titers in the serum further increased. Specifically, on Day 28, in the low-dose group, the mean titers of binding antibody against RBD of the ancestral and the Omicron BA. 1 strains reached 2,919,297 and 579,262, respectively. Finally, on Day 28 the high-dose group showed higher titer values (at >3,276,800 and 1,638,400, respectively) against both strains than the low-dose group, maintaining a good dose-dependent immune effect.

The above results demonstrate the mRNA-LNP vaccine stimulates high titers of antigen-specific binding antibodies indicating strong humoral immunogenicity of the vaccine; antibody titers elicited by the mRNA-LNP vaccine demonstrated dose-dependency and after the second injection, the titers of the binding antibody were further increased.

### SARS-CoV-2 Pseudovirus Neutralizing Antibodies:

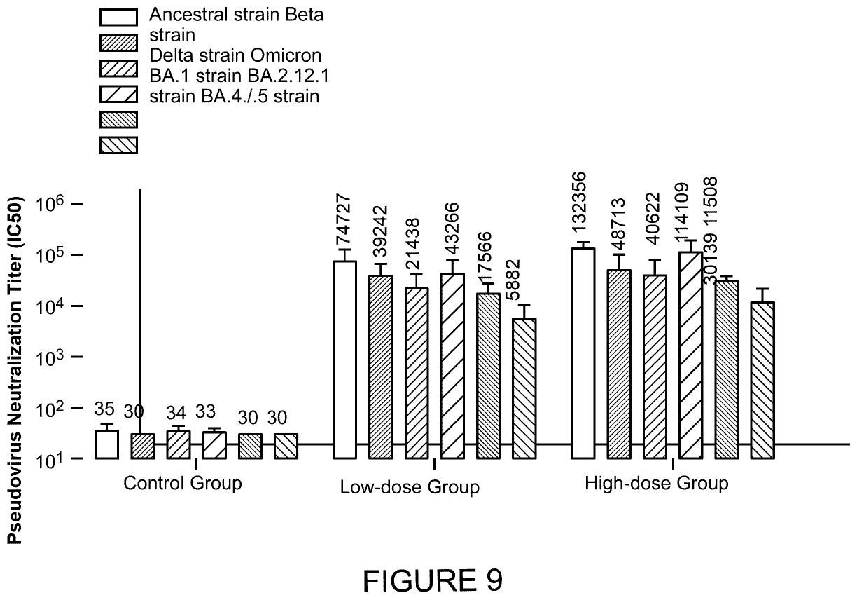

At the end point of the study (D28), which was 7 days after the boost immunization, serum was collected from the mice for neutralizing antibody testing. The neutralizing antibody titers against 6 SARS-CoV-2 pseudoviruses (the ancestral, Beta, Delta, Omicron BA.1, Omicron BA.2.12.1, and Omicron BA.4/.5) were determined. The results of neutralizing antibody titers (IC50, titers at which 50% of the pseudovirus is neutralized) are summarized in Table 4 and FIG. 9. Under this study conditions, strong neutralizing antibody titers against all six pseudoviruses were detected in all treated mice on Day 28.

TABLE 4
Serum neutralizing antibody titers (IC50)

| | | | | | Omicron | Omicron | Omicron |
|---|---|---|---|---|---|---|---|
| Groups | Animal ID | Ancestral | Beta | Delta | BA.1 | BA.2.12.1 | BA.4/5 |
| Control | 0514 | <30 | <30 | <30 | <30 | <30 | <30 |
| Group | 0515 | <30 | <30 | <30 | <30 | <30 | <30 |
| | 0538 | 56 | <30 | 50 | 42 | <30 | <30 |
| | 0539 | <30 | <30 | <30 | <30 | <30 | <30 |
| Low-dose | 0501 | 48,908 | 17,078 | 17,347 | 18,477 | 8,545 | 7,421 |
| Group | 0503 | 56,451 | 35,934 | 19,330 | 37,880 | 24,942 | 9,570 |
| | 0504 | 44,091 | 30,007 | 15,550 | 65,762 | 17,146 | 4,005 |
| | 0505 | 125,416 | 51,944 | 37,691 | 34,379 | 18,585 | 10,409 |
| | 0506 | 130,248 | 72,732 | 51,727 | 51,736 | 14,716 | 4,523 |
| | 0544 | 87,564 | 52,489 | 9,548 | 80,128 | 29,396 | 2,524 |
| High-dose | 0508 | 91,927 | 13,200 | 13,416 | 171,199 | 29,337 | 17,038 |
| Group | 0511 | 106,710 | 50,769 | 69,832 | 52,623 | 20,411 | 13,957 |
| | 0512 | 150,071 | 107,539 | 38,011 | 137,333 | 38,476 | 18,184 |
| | 0535 | 208,216 | 36,295 | 45,943 | 154,075 | 37,345 | 15,926 |
| | 0536 | 123,379 | 58,633 | 36,938 | 178,756 | 26,028 | 8,065 |
| | 0537 | 142,155 | 87.121 | 74,348 | 64,784 | 33,466 | 4,183 |

The results show that the mRNA-LNP vaccine elicited significant neutralizing antibodies against 6 SARS-CoV-2 pseudoviruses including the ancestral, Beta, Delta, Omicron BA.1, Omicron BA.2.12.1, and Omicron BA.4/5 strain with a clear trend of dose-dependency.

Together, the antigen-specific antibody titers and neutralizing activities indicate the mRNA-LNP vaccine was capable of stimulating broadly protective humoral immunity against SARS-CoV-2 different variants of concern.

### T-Cell Response:

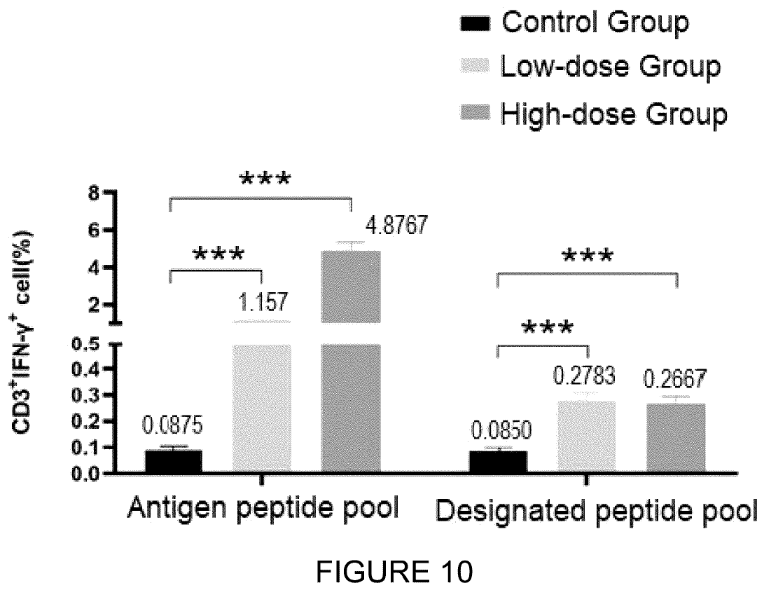

To evaluate the ability of the mRNA-LNP vaccine to stimulate cellular immunity, the percentages of IFN-γ producing T cells under antigen peptide pool (15mer peptides with 11 amino acid overlap covering the full sequence of the antigen) overstimulation were measured by ICS in splenocytes isolated from mice immunized with the mRNA-LNP vaccine. As shown in FIG. 10, on Day 28 in the low-dose group, the proportion of IFN-γ producing T-cells in total splenocytes was 1.152% upon stimulation by the antigen peptide pool and 0.278% upon stimulation by the designated peptide pool (15mer peptides with 11 amino acid overlap covering the L452R and F486V mutations, 7 peptides in total) just containing the peptides with L452R and F486V mutations, respectively. The high-dose group showed higher or equivalent percentages of IFN-γ producing T-cells than the low-dose group at 4.877% and 0.267%, respectively, demonstrating a trend of dose-dependency.

The results demonstrate: the mRNA-LNP vaccine could elicit significant cellular immune response in mice as demonstrated by significantly increased INF-γ producing T lymphocytes upon antigen peptide pool stimulation; the stimulated cellular immune response showed a trend of dose-dependency and the mRNA-LNP was able to elicit cellular immune response specific to Omicron BA.4/5 strains.

### Conclusion

The results of these examples demonstrate the following: immunization with the mRNA-LNP vaccine elicits a high titer of antigen specific binding antibodies against the SARS-CoV-2 ancestral strain and Omicron BA.1 strain; immunization with mRNA-LNP vaccine elicits strong pseudovirus neutralizing antibody titers against the ancestral strain, Beta strain, Delta strain, Omicron BA.1 strain, Omicron BA.2.12.1 strain, and Omicron BA.4/.5 strain, showing a significant cross-strain neutralization activities against the SARS-CoV-2 coronavirus variants of concern. Immunization with mRNA-LNP vaccine induces strong T cell responses as indicated by significantly elevated frequencies of T cells secreting IFN-γ, indicting enhanced cellular immune function of splenic T-lymphocytes of the immunized mice. Overall, mRNA-LNP not only elicits a strong cross strain humoral immune response against different SARS-CoV-2 variants of concern, but also generates a strong cellular immunity, demonstrating the mRNA-LNP vaccine as a broadly protective vaccine against SARS-CoV-2 primary infection and/or diseases.

### Example 11. In Vivo Immunogenicity Evaluation of Various mRNA-LNP Vaccine in Mice

This Example evaluates the in vivo effects of a mRNA vaccine that encodes virus antigens.

### Antigen-Specific Antibody Responses

Blood was collected from the retro-orbital sinus of immunized mice, and serum prepared. Antibody binding titers against SARS-CoV-2 spike protein RBD-His tag (RBD-BA.1) or SARS-CoV-2 Spike protein RBD-His & Avi Tag (RBD-WT) were quantified by enzyme-linked immunosorbent assay (ELISA). Briefly, 100 μL of 0.5 μg/mL RBD-BA.1 or RBD-WT antigen diluted in carbonate-bicarbonate buffer was pre-coated onto 96-well clear polystyrene microplate overnight at 4° C. After washing with PBS-T (0.05% Tween-20 in PBS) three times, the coated plates were blocked with 300 μL blocking buffer (15% normal goat serum and 2% bovine serum albumin in PBS-T) for 1 h at 37° C. Serum samples were 2-fold serially diluted in blocking buffer, transferred to the plates, and incubated for 1 h at 37° C. After washing, plates were incubated with HRP-conjugated rabbit anti-mouse IgG H+L for 1 h at 37° C. Plates were washed and incubated with TMB Single-Component Substrate solution for 7 min at 37° C., and the reaction was stopped with ELISA Stop Solution. Absorbance was read at 450 nm on a microplate reader and ELISA titers were determined using non-linear 4-parameter variable slope analysis in GraphPad Prism 8 software.

### Measurements of HAI Titers

The HAI procedure was derived from the WHO Manual for the laboratory diagnosis and virological surveillance of influenza. One volume of the aliquoted sera was incubated with 3 volumes of the Receptor Destroying Enzyme II (RDE) reconstituted in 0.85% NaCl overnight at 37° C., and heat inactivated the next day by incubating for 30 minutes at 56° C. RDE treated sera were stored at 4° C. Red blood cells (RBCs) were washed and brought to a final working concentration of 0.8%. Prior to the HAI assay, HAU testing (titration and back titration) was performed for each influenza virus used, with the serially (two-fold) diluted virus and 50 μL of RBCs in 96 well plate format. After mixing and 30 minutes incubation at room temperature, plates were tilted about 90 degrees. The agglutination and titer results were recorded and confirmed in back titration assay. For the HAI test, RDE treated sera were serially (two-fold) diluted using 96 well plates and mixed with the 8 HAU of virus and 50 μL of RBCs. After 30 minutes of incubation, plates were tilted, and results were recorded.

### Results

Preparation and Characterization of mRNA-LNP Vaccine

After thawing, the appearance of this mRNA-LNP vaccine was a slightly milky white dispersion, which is the typical appearance of an LNP-mRNA product.

In addition to appearance, the key drug characteristics of LNP-mRNA vaccines (different structures) were measured and are shown in Table 5. The range of particle sizes with four different structures was 106 nm to 124 nm and the purity was approximately 95%=2%.

TABLE 5
Representative pharmaceutical characteristics of mRNA-LNPs

| | Particle | Poly- | | Concentra- |
|---|---|---|---|---|
| Formulation | size | dispersity | mRNA | tion |
| description | (nm) | index | purity | (μg/mL) |
| mRNA-LNP (structure-1) | 124 | 0.104 | 93% | 34.1 |
| mRNA-LNP (structure-2) | 106 | 0.140 | 96.4% | 45.9 |
| mRNA-LNP (structure-3) | 116 | 0.092 | 94.4% | 38.3 |
| mRNA-LNP (structure-4) | 108 | 0.163 | 95.8% | 41.2 |

Efficacy of mRNA-LNP Vaccine (Different Structures) in Mice

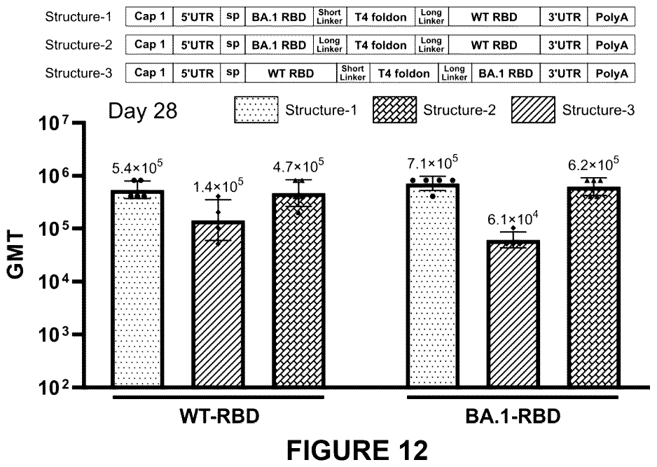

The efficacy of three different structures (Structure 1-3) for mRNA-LNP vaccines were compared in vivo. Compared with the original rigid short linker (SEQ ID NO. 5) of Structure-1, a flexible long linker (SEQ ID NO. 6) in Structure-2 connects BA.1 RBD (first antigen) (SEQ ID NO. 3) with WT RBD (second antigen) (SEQ ID NO. 4). Furthermore, in Structure-3, the positions of WT RBD (first antigen) and BA.1 RBD (second antigen) sequences are switched (FIG. 12).

For COVID-19 vaccines, in vivo studies were carried out by measuring the antibody response in BALB/c mice upon vaccination with an mRNA-LNP Vaccine (Structure-1, Structure-2, or Structure-3), which encode the WT RBD and BA.1 RBD antigens. Mice received a prime injection (Day 0) and a boost after three weeks (Day 21), the serum was collected in one week after the boost (Day 28).

Antibody titer is highly correlated with the protective effect and durability induced by the vaccine. Therefore, it is used as the efficacy read-out in this study and presented in the form of a geometric mean titer (GMT). FIG. 12 shows the GMT results of long or short linkers and the efficacy of different arrangements of GOI antigens. Compared with Structure-1, Structure-2 with a longer linker between first antigen and T4 foldon can also produce substantial antibodies against both antigens, which means that the efficacy of the structure is not subject to the length of linkers. Structure-2 can accommodate the longer, flexible sequence without reducing the expression of antigen and the titer of the elicited antibodies. Moreover, the GMT of Structure-3 for both WT-RBD and BA.1-RBD has a similar antibody levels to that of Structure-1. This suggests that the change in GOI antigen location arrangements within the design does not influence the final protein expression and the elicited immune protection of antibodies. Thus, the designs of Structure-2 and Structure-3 have a wide compatibility for internal components, such as different linkers and GOI location arrangements for different virus variants.

### Strain Specific Mouse Serum HAI Titers

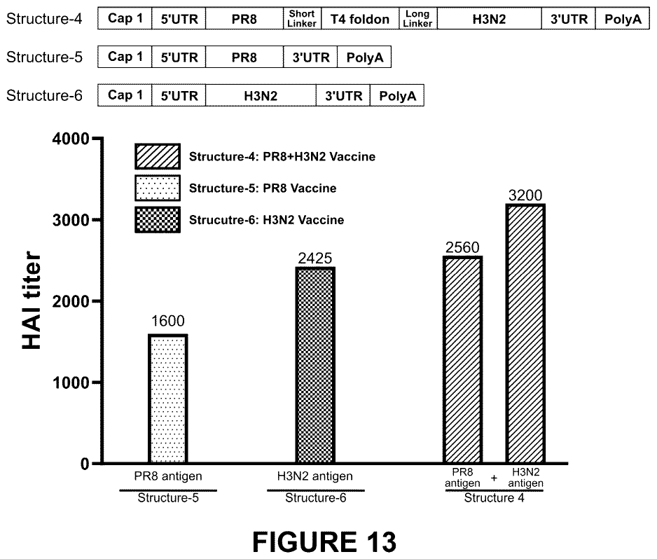

To prove the compatibility of this new structure platform, Structure-4 was designed, which encodes antigens of the PR8 and H3N2 virus for Flu vaccine instead of coronavirus. Three constructs of Flu vaccines were designed to evaluate the HAI titer levels including a conventional structure for PR8 vaccine (Structure-5), H3N2 vaccine (Structure-6), and Structure-4 which combine PR8 antigen (SEQ ID NO. 7) and H3N2 antigen (SEQ ID NO. 8) (FIG. 13). Different from the schematics of coronavirus sequence, signal peptides (sp) are not shown in the schematics of the constructs because they are within the PR8 or H3N2 antigen sequences.

After vaccinations on day 0 and day 21, mouse sera was collected on Day 28 and were treated with RDE and incubated with the respective viral antigens and RBCs to determine the level of hemagglutination inhibition (HAI), the result is shown in FIG. 13. The PR8 vaccine (Structure-5) and H3N2 vaccine (Structure-6) have a conventional mRNA structure (FIG. 13), which are the positive controls for PR8 antigen and H3N2 antigen at the relatively lower HAI titers of 1600 and 2425, respectively. The third design, Structure-4, encodes both PR8 and H3N2 antigens in the same vector, and had an HAI titer of 2560 and 3200 against PR8 and H3N2 antigen, respectively (FIG. 13). This result indicates that Structure-4 has a good compatibility for different antigen GOIs without affecting individual antigen expression. In fact, the HAI titers for both PR8 and H3N2 are higher than the ones observed for the conventional single GOI mRNA vector. Thus, Structure-4, a multivariance RNA vector, can be used for the expression of different virus variant antigens, which would not reduce the expression of individual antigens within this new structure.

SEQUENCE LISTING

| | SEQ ID | |
|---|---|---|
| Name | NO | Sequence |
| Trimerization | SEQ ID | GCCATCGGCGGCTACATCCCCGAGGCCCCCAGAGACGGC |
| sequence T4 | NO: 1 | CAGGCCTACGTGAGAAAGGACGGCGAGTGGGTGCTGCTG |
| Foldon (DNA) | | AGCACCTTCCT |
| Signal peptide | SEQ ID | ATGCCTATGGGCAGCCTGCAGCCCCTGGCCACCCTGTACC |
| CD5 (DNA) | NO: 2 | TGCTGGGCATGCTGGTGGCCAGCGTGCTGGCC |
| BA.1 strain RBD | SEQ ID | AACATCACCAACCTCTGCCCCTTCGACGAGGTGTTCAACG |
| (DNA) | NO: 3 | CCACCAGATTCGCCAGCGTCTACGCCTGGAACCGGAAGCG |
| | | GATCAGCAATTGCGTGGCCGACTACAGCGTGCTGTACAAC |
| | | CTCGCCCCTTTCTTCACCTTCAAGTGCTACGGCGTGTCCCC |
| | | TACCAAGCTGAACGATCTGTGCTTCACCAACGTGTACGCC |
| | | GACAGCTTCGTGATCAGGGGCGACGAAGTGCGACAGATC |
| | | GCCCCAGGACAGACAGGAAACATCGCCGATTACAACTAC |
| | | AAGCTGCCCGACGACTTCACCGGTTGCGTGATCGCTTGGA |
| | | ACAGCAACAAGCTGGACAGCAAGGTCTCCGGCAACTACA |
| | | ACTACCGGTACCGGCTGTTCCGGAAGTCCAACCTGAAGCC |
| | | CTTCGAGCGGGACATCAGCACAGAGATCTACCAGGCCGG |
| | | CAACAAACCTTGTAACGGCGTGGCGGGCgTCAACTGCTAC |
| | | TTCCCTCTGCGGAGCTACAGCTTCCGGCCTACATACGGCG |
| | | TGGGACACCAGCCTTACAGGGTGGTGGTGCTGAGCTTCGA |
| | | ACTGCTGCACGCTCCAGCCACCGTCTGCGGACCTAAAAAG |
| | | AGCACCAACCTGGTGAAGAACAAGTGCGTGAATTTC |
| WT strain RBD | SEQ ID | AACATCACCAACCTGTGCCCCTTCGGCGAGGTGTTCAACG |
| (DNA) | NO: 4 | CCACCAGATTCGCCAGCGTGTACGCCTGGAACAGAAAGA |
| | | GAATCAGCAACTGCGTGGCCGACTACAGCGTGCTGTACAA |
| | | CAGCGCCAGCTTCAGCACCTTCAAGTGCTACGGCGTGAGC |
| | | CCCACCAAGCTGAACGACCTGTGCTTCACCAACGTGTACG |
| | | CCGACAGCTTCGTGATCAGAGGCGACGAGGTGAGACAGA |
| | | TCGCCCCCGGCCAGACCGGCAAGATCGCCGACTACAACTA |
| | | CAAGCTGCCCGACGACTTCACCGGCTGCGTGATCGCCTGG |
| | | AACAGCAACAACCTGGACAGCAAGGTGGGCGGCAACTAC |
| | | AACTACCTGTACAGACTGTTCAGAAAGAGCAACCTGAAG |
| | | CCCTTCGAGAGAGACATCAGCACCGAGATCTACCAGGCC |
| | | GGCAGCACCCCCTGCAACGGCGTGGAGGGCTTCAACTGCT |
| | | ACTTCCCCCTGCAGAGCTACGGCTTCCAGCCCACCAACGG |
| | | CGTGGGCTACCAGCCCTACAGAGTGGTGGTGCTGAGCTTC |
| | | GAGCTGCTGCACGCCCCCGCCACCGTGTGCGGCCCCAAGA |
| | | AGTCCACCAACCTGGTGAAGAACAAGTGCGTGAACTTC |
| Short Linker | SEQ ID | GGCGGCAGCGGC |
| (DNA sequence) | NO.: 5 | |
| Long Linker | SEQ ID | GGCGGCAGCCTGGGCGGCGGCGGCAGCGGCAGC |
| (DNA sequence)r | NO.: 6 | |
| PR8 antigen | SEQ ID | ATGAAGGCCAACCTGCTGGTCCTGCTGTGCGCCCTGGCCG |
| (DNA sequence) | NO.: 7 | CTGCCGACGCCGACACCATCTGCATCGGCTACCACGCCAA |
| | | CAACAGCACCGACACCGTGGACACCGTGCTGGAGAAGAA |
| | | CGTGACCGTGACCCACAGCGTGAACCTGCTGGAGGACAG |
| | | CCACAACGGCAAGCTGTGCAGACTGAAGGGCATCGCCCCC |
| | | CTGCAGCTGGGCAAGTGCAACATCGCCGGCTGGCTGCTGG |
| | | GCAACCCCGAGTGCGACCCCCTGCTGCCCGTGAGAAGCTG |
| | | GAGCTACATCGTGGAGACCCCCAACAGCGAGAACGGCATC |
| | | TGCTACCCCGGCGACTTCATCGACTACGAGGAGCTGAGAG |
| | | AGCAGCTGAGCAGCGTGAGCAGCTTCGAGAGATTCGAGAT |
| | | CTTCCCCAAGGAGAGCAGCTGGCCCAACCACAACACCAA |
| | | CGGCGTGACCGCCGCCTGCAGCCACGAGGGCAAGAGCAG |
| | | CTTCTACAGAAACCTGCTGTGGCTGACCGAGAAGGAGGGC |
| | | AGCTACCCCAAGCTGAAGAACTCCTATGTGAACAAGAAGG |
| | | GCAAAGAGGTGCTGGTGCTGTGGGGCATCCACCACCCCCC |
| | | CAACAGCAAGGAGCAGCAGAACCTGTATCAGAACGAGAA |
| | | CGCCTACGTGAGCGTGGTGACAAGCAACTACAACAGAAG |
| | | ATTCACCCCCGAGATCGCCGAGAGACCCAAGGTGAGAGAC |
| | | CAAGCCGGCAGAATGAACTACTACTGGACCCTGCTGAAGC |
| | | CCGGCGACACCATCATCTTCGAGGCCAACGGCAACCTGAT |
| | | CGCCCCTATGTACGCCTTCGCCCTGAGCAGAGGCTTCGGC |
| | | AGCGGCATCATCACAAGCAACGCTAGCATGCACGAGTGCA |
| | | ACACCAAGTGTCAGACCCCCCTGGGCGCCATCAACAGCAG |
| | | CCTGCCCTATCAGAACATCCACCCCGTGACCATCGGCGAGT |
| | | GCCCCAAGTACGTGAGAAGCGCCAAGCTGAGAATGGTGA |
| | | CCGGCCTGAGAAACATCCCTAGCATTCAGAGCAGAGGCCT |
| | | GTTCGGCGCCATCGCCGGCTTCATCGAGGGCGGCTGGACC |
| | | GGCATGATCGACGGCTGGTACGGCTACCACCATCAGAACG |
| | | AGCAAGGCAGCGGCTACGCCGCCGATCAAAAGAGCACAC |
| | | AGAACGCCATCAACGGCATCACCAACAAGGTGAACACCGT |
| | | GATCGAGAAGATGAACATTCAGTTCACCGCCGTGGGCAAG |
| | | GAGTTCAACAAGCTGGAGAAGAGAATGGAGAACCTCAAC |
| | | AAGAAGGTGGACGACGGCTTCCTGGACATCTGGACCTACA |
| | | ACGCCGAGCTGCTGGTGCTGCTGGAGAACGAGAGAACCC |
| | | TGGACTTCCACGACAGCAACGTGAAGAACCTGTACGAGA |
| | | AGGTCAAGAGCCAACTGAAGAACAATGCCAAGGAGATCG |
| | | GCAACGGCTGCTTCGAGTTCTACCACAAGTGCGACAACGA |
| | | GTGCATGGAGAGCGTGAGAAACGGCACCTACGACTACCCC |
| | | AAGTACAGCGAGGAGAGCAAGCTGAACAGAGAGAAGGTC |
| | | GACGGCGTGAAGCTGGAGAGCATGGGCATCTATCAGATC |
| H3N2 antigen | NO.: 8 | AAGACAATCATCGCCCTGAGCCACATCCTGTGCCTGGTGTT |
| (DNA sequence) | SEQ ID | CGCTCAGAAGCTGCCCGGCAACGACAACAGCACCGCCAC |
| | | CCTGTGCCTGGGCCACCACGCCGTGCCCAACGGCACCATC |
| | | GTGAAAACAATCACCAACGATCAGATCGAGGTGACCAACG |
| | | CCACCGAGCTGGTGCAGAACAGCAGCATCGGCGAGATCTG |
| | | CGACAGCCCCCATCAGATCCTGGACGGCGAGAACTGCACC |
| | | CTGATCGACGCTCTGCTGGGCGATCCTCAGTGCGACGGCT |
| | | TTCAGAACAAGAAGTGGGACCTGTTCGTGGAGAGAAGCA |
| | | AGGCCTACAGCAACTGCTACCCCTACGATGTCCCCGACTAC |
| | | GCTAGCCTGAGAAGCCTGGTGGCTAGCAGCGGCACCCTGG |
| | | AGTTCAACAACGAGAGCTTCAACTGGACCGGCGTGACAC |
| | | AGAACGGCACAAGCAGCGCCTGCATCAGAAGAAGCAACA |
| | | ACAGCTTCTTCAGCAGACTGAACTGGCTGACACAGCTGAA |
| | | CTTCAAGTACCCCGCCCTGAACGTGACCATGCCCAACAAC |
| | | GAGCAGTTCGACAAGCTGTACATCTGGGGCGTGCACCACC |
| | | CCGTGACCGACAAGGATCAGATCTTCCTGTACGCTCAGAG |
| | | CAGCGGCAGAATCACCGTGAGCACCAAGAGAAGCCAACA |
| | | AGCCGTGATCCCCAACATCGGCTACAGACCTAGAATCAGA |
| | | AACATCCCTAGCAGAATCAGCATCTACTGGACCATCGTGAA |
| | | GCCCGGCGACATCCTGCTGATCAACAGCACCGGCAACCTG |
| | | ATCGCCCCTAGAGGCTACTTCAAGATCAGAAGCGGCAAGA |
| | | GCAGCATCATGAGAAGCGACGCCCCCATCGGCAAGTGCAA |
| | | CAGCGAGTGCATCACCCCCAACGGCAGCATCCCCAATGAT |
| | | AAGCCCTTTCAGAACGTGAACAGAATCACCTACGGCGCCT |
| | | GCCCTAGATACGTGAAGCAGAGCACCCTGAAGCTGGCCAC |
| | | CGGCATGAGAAACGTGCCCGAGAAGCAGACAAGAGGCAT |
| | | CTTCGGCGCCATCGCCGGCTTCATCGAGAACGGCTGGGAG |
| | | GGCATGGTGGACGGCTGGTACGGCTTCAGACATCAGAACA |
| | | GCGAGGGCAGAGGCCAAGCCGCCGACCTCAAGAGCACCC |
| | | AAGCCGCCATCGATCAGATCAACGGCAAGCTGAACAGACT |
| | | GATCGGCAAGACCAACGAGAAGTTCCATCAGATCGAGAAG |
| | | GAGTTCAGCGAGGTGGAGGGCAGAATCCAAGACCTGGAG |
| | | AAGTACGTGGAGGACACCAAGATCGACCTGTGGAGCTACA |
| | | ACGCCGAGCTGCTGGTGGCCCTGGAGAATCAGCACACCAT |
| | | CGACCTGACCGACAGCGAGATGAACAAGCTGTTCGAGAA |
| | | GACCAAGAAGCAGCTGAGAGAGAACGCCGAGGACATGGG |
| | | CAACGGCTGCTTCAAGATCTACCACAAGTGCGACAACGCC |
| | | TGCATCGGCAGCATCAGAAACGGCACCTACGACCACGACG |
| | | TGTACAGAGACGAGGCCCTGAACAACAGATTTCAGATCAA |
| | | GGGCGTGTGA |
| Flu A-strain full | SEQ ID | ATGAAGGCCAACCTGCTGGTCCTGCTGTGCGCCCTGGCCG |
| construct | NO: 12 | CTGCCGACGCCGACACCATCTGCATCGGCTACCACGCCAA |
| | | CAACAGCACCGACACCGTGGACACCGTGCTGGAGAAGAA |
| | | CGTGACCGTGACCCACAGCGTGAACCTGCTGGAGGACAG |
| | | CCACAACGGCAAGCTGTGCAGACTGAAGGGCATCGCCCC |
| | | CCTGCAGCTGGGCAAGTGCAACATCGCCGGCTGGCTGCTG |
| | | GGCAACCCCGAGTGCGACCCCCTGCTGCCCGTGAGAAGCT |
| | | GGAGCTACATCGTGGAGACCCCCAACAGCGAGAACGGCA |
| | | TCTGCTACCCCGGCGACTTCATCGACTACGAGGAGCTGAG |
| | | AGAGCAGCTGAGCAGCGTGAGCAGCTTCGAGAGATTCGA |
| | | GATCTTCCCCAAGGAGAGCAGCTGGCCCAACCACAACAC |
| | | CAACGGCGTGACCGCCGCCTGCAGCCACGAGGGCAAGAG |
| | | CAGCTTCTACAGAAACCTGCTGTGGCTGACCGAGAAGGA |
| | | GGGCAGCTACCCCAAGCTGAAGAACTCCTATGTGAACAA |
| | | GAAGGGCAAAGAGGTGCTGGTGCTGTGGGGCATCCACCA |
| | | CCCCCCCAACAGCAAGGAGCAGCAGAACCTGTATCAGAA |
| | | CGAGAACGCCTACGTGAGCGTGGTGACAAGCAACTACAA |
| | | CAGAAGATTCACCCCCGAGATCGCCGAGAGACCCAAGGT |
| | | GAGAGACCAAGCCGGCAGAATGAACTACTACTGGACCCT |
| | | GCTGAAGCCCGGCGACACCATCATCTTCGAGGCCAACGGC |
| | | AACCTGATCGCCCCTATGTACGCCTTCGCCCTGAGCAGAG |
| | | GCTTCGGCAGCGGCATCATCACAAGCAACGCTAGCATGCA |
| | | CGAGTGCAACACCAAGTGTCAGACCCCCCTGGGCGCCATC |
| | | AACAGCAGCCTGCCCTATCAGAACATCCACCCCGTGACCA |
| | | TCGGCGAGTGCCCCAAGTACGTGAGAAGCGCCAAGCTGA |
| | | GAATGGTGACCGGCCTGAGAAACATCCCTAGCATTCAGA |
| | | GCAGAGGCCTGTTCGGCGCCATCGCCGGCTTCATCGAGGG |
| | | CGGCTGGACCGGCATGATCGACGGCTGGTACGGCTACCAC |
| | | CATCAGAACGAGCAAGGCAGCGGCTACGCCGCCGATCAA |
| | | AAGAGCACACAGAACGCCATCAACGGCATCACCAACAAG |
| | | GTGAACACCGTGATCGAGAAGATGAACATTCAGTTCACCG |
| | | CCGTGGGCAAGGAGTTCAACAAGCTGGAGAAGAGAATGG |
| | | AGAACCTCAACAAGAAGGTGGACGACGGCTTCCTGGACA |
| | | TCTGGACCTACAACGCCGAGCTGCTGGTGCTGCTGGAGAA |
| | | CGAGAGAACCCTGGACTTCCACGACAGCAACGTGAAGAA |
| | | CCTGTACGAGAAGGTCAAGAGCCAACTGAAGAACAATGC |
| | | CAAGGAGATCGGCAACGGCTGCTTCGAGTTCTACCACAAG |
| | | TGCGACAACGAGTGCATGGAGAGCGTGAGAAACGGCACC |
| | | TACGACTACCCCAAGTACAGCGAGGAGAGCAAGCTGAAC |
| | | AGAGAGAAGGTCGACGGCGTGAAGCTGGAGAGCATGGGC |
| | | ATCTATCAGATCGGCGGCAGCGGCGCCATCGGCGGCTACA |
| | | TCCCCGAGGCCCCCAGAGACGGCCAGGCCTACGTGAGAA |
| | | AGGACGGCGAGTGGGTGCTGCTGAGCACCTTCCTGGGCG |
| | | GCAGCCTGGGCGGCGGCGGCAGCGGCAGCAAGACAATCA |
| | | TCGCCCTGAGCCACATCCTGTGCCTGGTGTTCGCTCAGAA |
| | | GCTGCCCGGCAACGACAACAGCACCGCCACCCTGTGCCTG |
| | | GGCCACCACGCCGTGCCCAACGGCACCATCGTGAAAACA |
| | | ATCACCAACGATCAGATCGAGGTGACCAACGCCACCGAG |
| | | CTGGTGCAGAACAGCAGCATCGGCGAGATCTGCGACAGC |
| | | CCCCATCAGATCCTGGACGGCGAGAACTGCACCCTGATCG |
| | | ACGCTCTGCTGGGCGATCCTCAGTGCGACGGCTTTCAGAA |
| | | CAAGAAGTGGGACCTGTTCGTGGAGAGAAGCAAGGCCTA |
| | | CAGCAACTGCTACCCCTACGATGTCCCCGACTACGCTAGC |
| | | CTGAGAAGCCTGGTGGCTAGCAGCGGCACCCTGGAGTTCA |
| | | ACAACGAGAGCTTCAACTGGACCGGCGTGACACAGAACG |
| | | GCACAAGCAGCGCCTGCATCAGAAGAAGCAACAACAGCT |
| | | TCTTCAGCAGACTGAACTGGCTGACACAGCTGAACTTCAA |
| | | GTACCCCGCCCTGAACGTGACCATGCCCAACAACGAGCA |
| | | GTTCGACAAGCTGTACATCTGGGGCGTGCACCACCCCGTG |
| | | ACCGACAAGGATCAGATCTTCCTGTACGCTCAGAGCAGCG |
| | | GCAGAATCACCGTGAGCACCAAGAGAAGCCAACAAGCCG |
| | | TGATCCCCAACATCGGCTACAGACCTAGAATCAGAAACAT |
| | | CCCTAGCAGAATCAGCATCTACTGGACCATCGTGAAGCCC |
| | | GGCGACATCCTGCTGATCAACAGCACCGGCAACCTGATCG |
| | | CCCCTAGAGGCTACTTCAAGATCAGAAGCGGCAAGAGCA |
| | | GCATCATGAGAAGCGACGCCCCCATCGGCAAGTGCAACA |
| | | GCGAGTGCATCACCCCCAACGGCAGCATCCCCAATGATAA |
| | | GCCCTTTCAGAACGTGAACAGAATCACCTACGGCGCCTGC |
| | | CCTAGATACGTGAAGCAGAGCACCCTGAAGCTGGCCACC |
| | | GGCATGAGAAACGTGCCCGAGAAGCAGACAAGAGGCATC |
| | | TTCGGCGCCATCGCCGGCTTCATCGAGAACGGCTGGGAGG |
| | | GCATGGTGGACGGCTGGTACGGCTTCAGACATCAGAACA |
| | | GCGAGGGCAGAGGCCAAGCCGCCGACCTCAAGAGCACCC |
| | | AAGCCGCCATCGATCAGATCAACGGCAAGCTGAACAGAC |
| | | TGATCGGCAAGACCAACGAGAAGTTCCATCAGATCGAGA |
| | | AGGAGTTCAGCGAGGTGGAGGGCAGAATCCAAGACCTGG |
| | | AGAAGTACGTGGAGGACACCAAGATCGACCTGTGGAGCT |
| | | ACAACGCCGAGCTGCTGGTGGCCCTGGAGAATCAGCACA |
| | | CCATCGACCTGACCGACAGCGAGATGAACAAGCTGTTCG |
| | | AGAAGACCAAGAAGCAGCTGAGAGAGAACGCCGAGGAC |
| | | ATGGGCAACGGCTGCTTCAAGATCTACCACAAGTGCGACA |
| | | ACGCCTGCATCGGCAGCATCAGAAACGGCACCTACGACC |
| | | ACGACGTGTACAGAGACGAGGCCCTGAACAACAGATTTC |
| | | AGATCAAGGGCGTGTGA |
| COVID-19 RBD | SEQ ID | ATGCCTATGGGCAGCCTGCAGCCCCTGGCCACCCTGTACC |
| full construct | NO: 13 | TGCTGGGCATGCTGGTGGCCAGCGTGCTGGCCGGCGGCAG |
| | | CGGCGGCGGCGGCAGCGGATCCAACATCACCAACCTCTG |
| | | CCCCTTCGACGAGGTGTTCAACGCCACCAGATTCGCCAGC |
| | | GTCTACGCCTGGAACCGGAAGCGGATCAGCAATTGCGTG |
| | | GCCGACTACAGCGTGCTGTACAACCTCGCCCCTTTCTTCA |
| | | CCTTCAAGTGCTACGGCGTGTCCCCTACCAAGCTGAACGA |
| | | TCTGTGCTTCACCAACGTGTACGCCGACAGCTTCGTGATC |
| | | AGGGGCGACGAAGTGCGACAGATCGCCCCAGGACAGACA |
| | | GGAAACATCGCCGATTACAACTACAAGCTGCCCGACGACT |
| | | TCACCGGTTGCGTGATCGCTTGGAACAGCAACAAGCTGGA |
| | | CAGCAAGGTCTCCGGCAACTACAACTACCGGTACCGGCTG |
| | | TTCCGGAAGTCCAACCTGAAGCCCTTCGAGCGGGACATCA |
| | | GCACAGAGATCTACCAGGCCGGCAACAAACCTTGTAACG |
| | | GCGTGGCGGGCgTCAACTGCTACTTCCCTCTGCGGAGCTA |
| | | CAGCTTCCGGCCTACATACGGCGTGGGACACCAGCCTTAC |
| | | AGGGTGGTGGTGCTGAGCTTCGAACTGCTGCACGCTCCAG |
| | | CCACCGTCTGCGGACCTAAAAAGAGCACCAACCTGGTGA |
| | | AGAACAAGTGCGTGAATTTCGGCGGCAGCGGCGCCATCG |
| | | GCGGCTACATCCCCGAGGCCCCCAGAGACGGCCAGGCCT |
| | | ACGTGAGAAAGGACGGCGAGTGGGTGCTGCTGAGCACCT |
| | | TCCTGGGCGGCAGCCTGGGCGGCGGCGGCAGCGGCAGCA |
| | | ACATCACCAACCTGTGCCCCTTCGGCGAGGTGTTCAACGC |
| | | CACCAGATTCGCCAGCGTGTACGCCTGGAACAGAAAGAG |
| | | AATCAGCAACTGCGTGGCCGACTACAGCGTGCTGTACAAC |
| | | AGCGCCAGCTTCAGCACCTTCAAGTGCTACGGCGTGAGCC |
| | | CCACCAAGCTGAACGACCTGTGCTTCACCAACGTGTACGC |
| | | CGACAGCTTCGTGATCAGAGGCGACGAGGTGAGACAGAT |
| | | CGCCCCCGGCCAGACCGGCAAGATCGCCGACTACAACTA |
| | | CAAGCTGCCCGACGACTTCACCGGCTGCGTGATCGCCTGG |
| | | AACAGCAACAACCTGGACAGCAAGGTGGGCGGCAACTAC |
| | | AACTACCTGTACAGACTGTTCAGAAAGAGCAACCTGAAG |
| | | CCCTTCGAGAGAGACATCAGCACCGAGATCTACCAGGCC |
| | | GGCAGCACCCCCTGCAACGGCGTGGAGGGCTTCAACTGCT |
| | | ACTTCCCCCTGCAGAGCTACGGCTTCCAGCCCACCAACGG |
| | | CGTGGGCTACCAGCCCTACAGAGTGGTGGTGCTGAGCTTC |
| | | GAGCTGCTGCACGCCCCCGCCACCGTGTGCGGCCCCAAGA |
| | | AGTCCACCAACCTGGTGAAGAACAAGTGCGTGAACTTC |
| 5′ UTR (DNA | SEQ ID | GGATAGGCGGCGCATGAGAGAAGCCCAGACCAATTACCT |
| sequence) | NO: 14 | ACCCAAAGCCACC |
| 3′ UTR (DNA | SEQ ID | CGGCCGCATACAGCAGCAATTGGCAAGCTGCTTACATAGA |
| sequence) | NO: 15 | ACTCGCGGCGATTGGCATGCCGCCTTAAAATTTTTCTTTTC |
| | | TTTTCCGAATCGGATTTTGTTTTTAATATTTAAGCTT |
| PolyA | SEQ ID | AAAAAAAAAAAAAAAAAAAAAAAAAAAAAAAAAAAAAA |
| | NO.: 16 | AAAAAAAAAAAAAAAAAAAAAAAAAAAAAAAAAAAAAA |
| | | AAAAAAAAAAAAAAAAAAAAAAAAAAAAAAAAAAAAAA |
| | | AAAAAAAAAAA |
| Trimerization | SEQ ID | GCCAUCGGCGGCUACAUCCCCGAGGCCCCCAGAGACGGC |
| sequence T4 | NO.: 17 | CAGGCCUACGUGAGAAAGGACGGCGAGUGGGUGCUGCU |
| Foldon (RNA | | GAGCACCUUCCU |
| sequence) | | |
| Signal peptide | SEQ ID | AUGCCUAUGGGCAGCCUGCAGCCCCUGGCCACCCUGUAC |
| CD5 (RNA | NO.: 18 | CUGCUGGGCAUGCUGGUGGCCAGCGUGCUGGCC |
| sequence) | | |
| BA.1 strain RBD | SEQ ID | AACAUCACCAACCUCUGCCCCUUCGACGAGGUGUUCAAC |
| (RNA sequence) | NO.: 19 | GCCACCAGAUUCGCCAGCGUCUACGCCUGGAACCGGAAG |
| | | CGGAUCAGCAAUUGCGUGGCCGACUACAGCGUGCUGUA |
| | | CAACCUCGCCCCUUUCUUCACCUUCAAGUGCUACGGCGU |
| | | GUCCCCUACCAAGCUGAACGAUCUGUGCUUCACCAACGU |
| | | GUACGCCGACAGCUUCGUGAUCAGGGGCGACGAAGUGC |
| | | GACAGAUCGCCCCAGGACAGACAGGAAACAUCGCCGAUU |
| | | ACAACUACAAGCUGCCCGACGACUUCACCGGUUGCGUGA |
| | | UCGCUUGGAACAGCAACAAGCUGGACAGCAAGGUCUCC |
| | | GGCAACUACAACUACCGGUACCGGCUGUUCCGGAAGUCC |
| | | AACCUGAAGCCCUUCGAGCGGGACAUCAGCACAGAGAUC |
| | | UACCAGGCCGGCAACAAACCUUGUAACGGCGUGGCGGGC |
| | | gUCAACUGCUACUUCCCUCUGCGGAGCUACAGCUUCCGG |
| | | CCUACAUACGGCGUGGGACACCAGCCUUACAGGGUGGU |
| | | GGUGCUGAGCUUCGAACUGCUGCACGCUCCAGCCACCGU |
| | | CUGCGGACCUAAAAAGAGCACCAACCUGGUGAAGAACA |
| | | AGUGCGUGAAUUUC |
| WT strain RBD | SEQ ID | AACAUCACCAACCUGUGCCCCUUCGGCGAGGUGUUCAAC |
| (RNA sequence) | NO.: 20 | GCCACCAGAUUCGCCAGCGUGUACGCCUGGAACAGAAAG |
| | | AGAAUCAGCAACUGCGUGGCCGACUACAGCGUGCUGUA |
| | | CAACAGCGCCAGCUUCAGCACCUUCAAGUGCUACGGCGU |
| | | GAGCCCCACCAAGCUGAACGACCUGUGCUUCACCAACGU |
| | | GUACGCCGACAGCUUCGUGAUCAGAGGCGACGAGGUGA |
| | | GACAGAUCGCCCCCGGCCAGACCGGCAAGAUCGCCGACU |
| | | ACAACUACAAGCUGCCCGACGACUUCACCGGCUGCGUGA |
| | | UCGCCUGGAACAGCAACAACCUGGACAGCAAGGUGGGC |
| | | GGCAACUACAACUACCUGUACAGACUGUUCAGAAAGAG |
| | | CAACCUGAAGCCCUUCGAGAGAGACAUCAGCACCGAGAU |
| | | CUACCAGGCCGGCAGCACCCCCUGCAACGGCGUGGAGGG |
| | | CUUCAACUGCUACUUCCCCCUGCAGAGCUACGGCUUCCA |
| | | GCCCACCAACGGCGUGGGCUACCAGCCCUACAGAGUGGU |
| | | GGUGCUGAGCUUCGAGCUGCUGCACGCCCCCGCCACCGU |
| | | GUGCGGCCCCAAGAAGUCCACCAACCUGGUGAAGAACAA |
| | | GUGCGUGAACUUC |
| Long Linker | SEQ ID | GGCGGCAGCCUGGGCGGCGGCGGCAGCGGCAGC |
| (RNA sequence) | NO.: 21 | |
| PR8 antigen | SEQ ID | AUGAAGGCCAACCUGCUGGUCCUGCUGUGCGCCCUGGCC |
| (RNA sequence) | NO.: 22 | GCUGCCGACGCCGACACCAUCUGCAUCGGCUACCACGCC |
| | | AACAACAGCACCGACACCGUGGACACCGUGCUGGAGAAG |
| | | AACGUGACCGUGACCCACAGCGUGAACCUGCUGGAGGAC |
| | | AGCCACAACGGCAAGCUGUGCAGACUGAAGGGCAUCGCC |
| | | CCCCUGCAGCUGGGCAAGUGCAACAUCGCCGGCUGGCUG |
| | | CUGGGCAACCCCGAGUGCGACCCCCUGCUGCCCGUGAGA |
| | | AGCUGGAGCUACAUCGUGGAGACCCCCAACAGCGAGAAC |
| | | GGCAUCUGCUACCCCGGCGACUUCAUCGACUACGAGGAG |
| | | CUGAGAGAGCAGCUGAGCAGCGUGAGCAGCUUCGAGAG |
| | | AUUCGAGAUCUUCCCCAAGGAGAGCAGCUGGCCCAACCA |
| | | CAACACCAACGGCGUGACCGCCGCCUGCAGCCACGAGGG |
| | | CAAGAGCAGCUUCUACAGAAACCUGCUGUGGCUGACCG |
| | | AGAAGGAGGGCAGCUACCCCAAGCUGAAGAACUCCUAU |
| | | GUGAACAAGAAGGGCAAAGAGGUGCUGGUGCUGUGGGG |
| | | CAUCCACCACCCCCCCAACAGCAAGGAGCAGCAGAACCU |
| | | GUAUCAGAACGAGAACGCCUACGUGAGCGUGGUGACAA |
| | | GCAACUACAACAGAAGAUUCACCCCCGAGAUCGCCGAGA |
| | | GACCCAAGGUGAGAGACCAAGCCGGCAGAAUGAACUAC |
| | | UACUGGACCCUGCUGAAGCCCGGCGACACCAUCAUCUUC |
| | | GAGGCCAACGGCAACCUGAUCGCCCCUAUGUACGCCUUC |
| | | GCCCUGAGCAGAGGCUUCGGCAGCGGCAUCAUCACAAGC |
| | | AACGCUAGCAUGCACGAGUGCAACACCAAGUGUCAGACC |
| | | CCCCUGGGCGCCAUCAACAGCAGCCUGCCCUAUCAGAAC |
| | | AUCCACCCCGUGACCAUCGGCGAGUGCCCCAAGUACGUG |
| | | AGAAGCGCCAAGCUGAGAAUGGUGACCGGCCUGAGAAA |
| | | CAUCCCUAGCAUUCAGAGCAGAGGCCUGUUCGGCGCCAU |
| | | CGCCGGCUUCAUCGAGGGCGGCUGGACCGGCAUGAUCGA |
| | | CGGCUGGUACGGCUACCACCAUCAGAACGAGCAAGGCAG |
| | | CGGCUACGCCGCCGAUCAAAAGAGCACACAGAACGCCAU |
| | | CAACGGCAUCACCAACAAGGUGAACACCGUGAUCGAGA |
| | | AGAUGAACAUUCAGUUCACCGCCGUGGGCAAGGAGUUC |
| | | AACAAGCUGGAGAAGAGAAUGGAGAACCUCAACAAGAA |
| | | GGUGGACGACGGCUUCCUGGACAUCUGGACCUACAACGC |
| | | CGAGCUGCUGGUGCUGCUGGAGAACGAGAGAACCCUGG |
| | | ACUUCCACGACAGCAACGUGAAGAACCUGUACGAGAAG |
| | | GUCAAGAGCCAACUGAAGAACAAUGCCAAGGAGAUCGG |
| | | CAACGGCUGCUUCGAGUUCUACCACAAGUGCGACAACGA |
| | | GUGCAUGGAGAGCGUGAGAAACGGCACCUACGACUACC |
| | | CCAAGUACAGCGAGGAGAGCAAGCUGAACAGAGAGAAG |
| | | GUCGACGGCGUGAAGCUGGAGAGCAUGGGCAUCUAUCA |
| | | GAUC |
| H3N2 antigen | SEQ ID | AAGACAAUCAUCGCCCUGAGCCACAUCCUGUGCCUGGUG |
| (RNA sequence) | NO.: 23 | UUCGCUCAGAAGCUGCCCGGCAACGACAACAGCACCGCC |
| | | ACCCUGUGCCUGGGCCACCACGCCGUGCCCAACGGCACC |
| | | AUCGUGAAAACAAUCACCAACGAUCAGAUCGAGGUGAC |
| | | CAACGCCACCGAGCUGGUGCAGAACAGCAGCAUCGGCGA |
| | | GAUCUGCGACAGCCCCCAUCAGAUCCUGGACGGCGAGAA |
| | | CUGCACCCUGAUCGACGCUCUGCUGGGCGAUCCUCAGUG |
| | | CGACGGCUUUCAGAACAAGAAGUGGGACCUGUUCGUGG |
| | | AGAGAAGCAAGGCCUACAGCAACUGCUACCCCUACGAUG |
| | | UCCCCGACUACGCUAGCCUGAGAAGCCUGGUGGCUAGCA |
| | | GCGGCACCCUGGAGUUCAACAACGAGAGCUUCAACUGG |
| | | ACCGGCGUGACACAGAACGGCACAAGCAGCGCCUGCAUC |
| | | AGAAGAAGCAACAACAGCUUCUUCAGCAGACUGAACUG |
| | | GCUGACACAGCUGAACUUCAAGUACCCCGCCCUGAACGU |
| | | GACCAUGCCCAACAACGAGCAGUUCGACAAGCUGUACAU |
| | | CUGGGGCGUGCACCACCCCGUGACCGACAAGGAUCAGAU |
| | | CUUCCUGUACGCUCAGAGCAGCGGCAGAAUCACCGUGAG |
| | | CACCAAGAGAAGCCAACAAGCCGUGAUCCCCAACAUCGG |
| | | CUACAGACCUAGAAUCAGAAACAUCCCUAGCAGAAUCA |
| | | GCAUCUACUGGACCAUCGUGAAGCCCGGCGACAUCCUGC |
| | | UGAUCAACAGCACCGGCAACCUGAUCGCCCCUAGAGGCU |
| | | ACUUCAAGAUCAGAAGCGGCAAGAGCAGCAUCAUGAGA |
| | | AGCGACGCCCCCAUCGGCAAGUGCAACAGCGAGUGCAUC |
| | | ACCCCCAACGGCAGCAUCCCCAAUGAUAAGCCCUUUCAG |
| | | AACGUGAACAGAAUCACCUACGGCGCCUGCCCUAGAUAC |
| | | GUGAAGCAGAGCACCCUGAAGCUGGCCACCGGCAUGAG |
| | | AAACGUGCCCGAGAAGCAGACAAGAGGCAUCUUCGGCG |
| | | CCAUCGCCGGCUUCAUCGAGAACGGCUGGGAGGGCAUG |
| | | GUGGACGGCUGGUACGGCUUCAGACAUCAGAACAGCGA |
| | | GGGCAGAGGCCAAGCCGCCGACCUCAAGAGCACCCAAGC |
| | | CGCCAUCGAUCAGAUCAACGGCAAGCUGAACAGACUGA |
| | | UCGGCAAGACCAACGAGAAGUUCCAUCAGAUCGAGAAG |
| | | GAGUUCAGCGAGGUGGAGGGCAGAAUCCAAGACCUGGA |
| | | GAAGUACGUGGAGGACACCAAGAUCGACCUGUGGAGCU |
| | | ACAACGCCGAGCUGCUGGUGGCCCUGGAGAAUCAGCACA |
| | | CCAUCGACCUGACCGACAGCGAGAUGAACAAGCUGUUCG |
| | | AGAAGACCAAGAAGCAGCUGAGAGAGAACGCCGAGGAC |
| | | AUGGGCAACGGCUGCUUCAAGAUCUACCACAAGUGCGA |
| | | CAACGCCUGCAUCGGCAGCAUCAGAAACGGCACCUACGA |
| | | CCACGACGUGUACAGAGACGAGGCCCUGAACAACAGAU |
| | | UUCAGAUCAAGGGCGUGUGA |
| 5′ UTR (RNA | SEQ ID | GGAUAGGCGGCGCAUGAGAGAAGCCCAGACCAAUUACC |
| sequence) | NO.: 24 | UACCCAAAGCCACC |
| 3′ UTR (RNA | SEQ ID | CGGCCGCAUACAGCAGCAAUUGGCAAGCUGCUUACAUA |
| sequence) | NO.: 25 | GAACUCGCGGCGAUUGGCAUGCCGCCUUAAAAUUUUUC |
| | | UUUUCUUUUCCGAAUCGGAUUUUGUUUUUAAUAUUUAA |
| | | GCUU |
| Structure 1 (RNA | SEQ ID | GGAUAGGCGGCGCAUGAGAGAAGCCCAGACCAAUUACC |
| sequence) | NO.: 26 | UACCCAAAGCCACCAUGCCUAUGGGCAGCCUGCAGCCCC |
| | | UGGCCACCCUGUACCUGCUGGGCAUGCUGGUGGCCAGCG |
| | | UGCUGGCCGGCGGCAGCGGCGGCGGCGGCAGCGGAUCCA |
| | | ACAUCACCAACCUCUGCCCCUUCGACGAGGUGUUCAACG |
| | | CCACCAGAUUCGCCAGCGUCUACGCCUGGAACCGGAAGC |
| | | GGAUCAGCAAUUGCGUGGCCGACUACAGCGUGCUGUAC |
| | | AACCUCGCCCCUUUCUUCACCUUCAAGUGCUACGGCGUG |
| | | UCCCCUACCAAGCUGAACGAUCUGUGCUUCACCAACGUG |
| | | UACGCCGACAGCUUCGUGAUCAGGGGCGACGAAGUGCG |
| | | ACAGAUCGCCCCAGGACAGACAGGAAACAUCGCCGAUUA |
| | | CAACUACAAGCUGCCCGACGACUUCACCGGUUGCGUGAU |
| | | CGCUUGGAACAGCAACAAGCUGGACAGCAAGGUCUCCG |
| | | GCAACUACAACUACCGGUACCGGCUGUUCCGGAAGUCCA |
| | | ACCUGAAGCCCUUCGAGCGGGACAUCAGCACAGAGAUCU |
| | | ACCAGGCCGGCAACAAACCUUGUAACGGCGUGGCGGGCG |
| | | UCAACUGCUACUUCCCUCUGCGGAGCUACAGCUUCCGGC |
| | | CUACAUACGGCGUGGGACACCAGCCUUACAGGGUGGUG |
| | | GUGCUGAGCUUCGAACUGCUGCACGCUCCAGCCACCGUC |
| | | UGCGGACCUAAAAAGAGCACCAACCUGGUGAAGAACAA |
| | | GUGCGUGAAUUUCGGCGGCAGCGGCGCCAUCGGCGGCU |
| | | ACAUCCCCGAGGCCCCCAGAGACGGCCAGGCCUACGUGA |
| | | GAAAGGACGGCGAGUGGGUGCUGCUGAGCACCUUCCUG |
| | | GGCGGCAGCCUGGGCGGCGGCGGCAGCGGCAGCAACAUC |
| | | ACCAACCUGUGCCCCUUCGGCGAGGUGUUCAACGCCACC |
| | | AGAUUCGCCAGCGUGUACGCCUGGAACAGAAAGAGAAU |
| | | CAGCAACUGCGUGGCCGACUACAGCGUGCUGUACAACAG |
| | | CGCCAGCUUCAGCACCUUCAAGUGCUACGGCGUGAGCCC |
| | | CACCAAGCUGAACGACCUGUGCUUCACCAACGUGUACGC |
| | | CGACAGCUUCGUGAUCAGAGGCGACGAGGUGAGACAGA |
| | | UCGCCCCCGGCCAGACCGGCAAGAUCGCCGACUACAACU |
| | | ACAAGCUGCCCGACGACUUCACCGGCUGCGUGAUCGCCU |
| | | GGAACAGCAACAACCUGGACAGCAAGGUGGGCGGCAAC |
| | | UACAACUACCUGUACAGACUGUUCAGAAAGAGCAACCU |
| | | GAAGCCCUUCGAGAGAGACAUCAGCACCGAGAUCUACCA |
| | | GGCCGGCAGCACCCCCUGCAACGGCGUGGAGGGCUUCAA |
| | | CUGCUACUUCCCCCUGCAGAGCUACGGCUUCCAGCCCAC |
| | | CAACGGCGUGGGCUACCAGCCCUACAGAGUGGUGGUGC |
| | | UGAGCUUCGAGCUGCUGCACGCCCCCGCCACCGUGUGCG |
| | | GCCCCAAGAAGUCCACCAACCUGGUGAAGAACAAGUGCG |
| | | UGAACUUCUGAUGACGGCCGCAUACAGCAGCAAUUGGC |
| | | AAGCUGCUUACAUAGAACUCGCGGCGAUUGGCAUGCCG |
| | | CCUUAAAAUUUUUCUUUUCUUUUCCGAAUCGGAUUUUG |
| | | UUUUUAAUAUUUAAGCUUAAAAAAAAAAAAAAAAAAAA |
| | | AAAAAAAAAAAAAAAAAAAAAAAAAAAAAAAAAAAAAA |
| | | AAAAAAAAAAAAAAAAAAAAAAAAAAAAAAAAAAAAAA |
| | | AAAAAAAAAAAAAAAAAAAAAAAAAAAAA |
| Structure 2 (RNA | SEQ ID | GGAUAGGCGGCGCAUGAGAGAAGCCCAGACCAAUUACC |
| sequence) | NO.: 27 | UACCCAAAGCCACCAUGCCUAUGGGCAGCCUGCAGCCCC |
| | | UGGCCACCCUGUACCUGCUGGGCAUGCUGGUGGCCAGCG |
| | | UGCUGGCCGGCGGCAGCGGCGGCGGCGGCAGCGGAUCCA |
| | | ACAUCACCAACCUCUGCCCCUUCGACGAGGUGUUCAACG |
| | | CCACCAGAUUCGCCAGCGUCUACGCCUGGAACCGGAAGC |
| | | GGAUCAGCAAUUGCGUGGCCGACUACAGCGUGCUGUAC |
| | | AACCUCGCCCCUUUCUUCACCUUCAAGUGCUACGGCGUG |
| | | UCCCCUACCAAGCUGAACGAUCUGUGCUUCACCAACGUG |
| | | UACGCCGACAGCUUCGUGAUCAGGGGCGACGAAGUGCG |
| | | ACAGAUCGCCCCAGGACAGACAGGAAACAUCGCCGAUUA |
| | | CAACUACAAGCUGCCCGACGACUUCACCGGUUGCGUGAU |
| | | CGCUUGGAACAGCAACAAGCUGGACAGCAAGGUCUCCG |
| | | GCAACUACAACUACCGGUACCGGCUGUUCCGGAAGUCCA |
| | | ACCUGAAGCCCUUCGAGCGGGACAUCAGCACAGAGAUCU |
| | | ACCAGGCCGGCAACAAACCUUGUAACGGCGUGGCGGGCG |
| | | UCAACUGCUACUUCCCUCUGCGGAGCUACAGCUUCCGGC |
| | | CUACAUACGGCGUGGGACACCAGCCUUACAGGGUGGUG |
| | | GUGCUGAGCUUCGAACUGCUGCACGCUCCAGCCACCGUC |
| | | UGCGGACCUAAAAAGAGCACCAACCUGGUGAAGAACAA |
| | | GUGCGUGAAUUUCGGCGGCAGCCUGGGCGGCGGCGGCA |
| | | GCGGCAGCGCCAUCGGCGGCUACAUCCCCGAGGCCCCCA |
| | | GAGACGGCCAGGCCUACGUGAGAAAGGACGGCGAGUGG |
| | | GUGCUGCUGAGCACCUUCCUGGGCGGCAGCCUGGGCGGC |
| | | GGCGGCAGCGGCAGCAACAUCACCAACCUGUGCCCCUUC |
| | | GGCGAGGUGUUCAACGCCACCAGAUUCGCCAGCGUGUAC |
| | | GCCUGGAACAGAAAGAGAAUCAGCAACUGCGUGGCCGA |
| | | CUACAGCGUGCUGUACAACAGCGCCAGCUUCAGCACCUU |
| | | CAAGUGCUACGGCGUGAGCCCCACCAAGCUGAACGACCU |
| | | GUGCUUCACCAACGUGUACGCCGACAGCUUCGUGAUCAG |
| | | AGGCGACGAGGUGAGACAGAUCGCCCCCGGCCAGACCGG |
| | | CAAGAUCGCCGACUACAACUACAAGCUGCCCGACGACUU |
| | | CACCGGCUGCGUGAUCGCCUGGAACAGCAACAACCUGGA |
| | | CAGCAAGGUGGGCGGCAACUACAACUACCUGUACAGAC |
| | | UGUUCAGAAAGAGCAACCUGAAGCCCUUCGAGAGAGAC |
| | | AUCAGCACCGAGAUCUACCAGGCCGGCAGCACCCCCUGC |
| | | AACGGCGUGGAGGGCUUCAACUGCUACUUCCCCCUGCAG |
| | | AGCUACGGCUUCCAGCCCACCAACGGCGUGGGCUACCAG |
| | | CCCUACAGAGUGGUGGUGCUGAGCUUCGAGCUGCUGCA |
| | | CGCCCCCGCCACCGUGUGCGGCCCCAAGAAGUCCACCAA |
| | | CCUGGUGAAGAACAAGUGCGUGAACUUCUGAUGACGGC |
| | | CGCAUACAGCAGCAAUUGGCAAGCUGCUUACAUAGAAC |
| | | UCGCGGCGAUUGGCAUGCCGCCUUAAAAUUUUUCUUUU |
| | | CUUUUCCGAAUCGGAUUUUGUUUUUAAUAUUUAAGCUU |
| | | AAAAAAAAAAAAAAAAAAAAAAAAAAAAAAAAAAAAAA |
| | | AAAAAAAAAAAAAAAAAAAAAAAAAAAAAAAAAAAAAA |
| | | AAAAAAAAAAAAAAAAAAAAAAAAAAAAAAAAAAAAAA |
| | | AAAAAAAAAAA |
| Structure 3 (RNA | SEQ ID | GGAUAGGCGGCGCAUGAGAGAAGCCCAGACCAAUUACC |
| sequence) | NO.: 28 | UACCCAAAGCCACCAUGCCUAUGGGCAGCCUGCAGCCCC |
| | | UGGCCACCCUGUACCUGCUGGGCAUGCUGGUGGCCAGCG |
| | | UGCUGGCCGGCGGCAGCGGCGGCGGCGGCAGCGGAUCCA |
| | | ACAUCACCAACCUGUGCCCCUUCGGCGAGGUGUUCAACG |
| | | CCACCAGAUUCGCCAGCGUGUACGCCUGGAACAGAAAGA |
| | | GAAUCAGCAACUGCGUGGCCGACUACAGCGUGCUGUAC |
| | | AACAGCGCCAGCUUCAGCACCUUCAAGUGCUACGGCGUG |
| | | AGCCCCACCAAGCUGAACGACCUGUGCUUCACCAACGUG |
| | | UACGCCGACAGCUUCGUGAUCAGAGGCGACGAGGUGAG |
| | | ACAGAUCGCCCCCGGCCAGACCGGCAAGAUCGCCGACUA |
| | | CAACUACAAGCUGCCCGACGACUUCACCGGCUGCGUGAU |
| | | CGCCUGGAACAGCAACAACCUGGACAGCAAGGUGGGCG |
| | | GCAACUACAACUACCUGUACAGACUGUUCAGAAAGAGC |
| | | AACCUGAAGCCCUUCGAGAGAGACAUCAGCACCGAGAUC |
| | | UACCAGGCCGGCAGCACCCCCUGCAACGGCGUGGAGGGC |
| | | UUCAACUGCUACUUCCCCCUGCAGAGCUACGGCUUCCAG |
| | | CCCACCAACGGCGUGGGCUACCAGCCCUACAGAGUGGUG |
| | | GUGCUGAGCUUCGAGCUGCUGCACGCCCCCGCCACCGUG |
| | | UGCGGCCCCAAGAAGUCCACCAACCUGGUGAAGAACAAG |
| | | UGCGUGAACUUCGGCGGCAGCGGCGCCAUCGGCGGCUAC |
| | | AUCCCCGAGGCCCCCAGAGACGGCCAGGCCUACGUGAGA |
| | | AAGGACGGCGAGUGGGUGCUGCUGAGCACCUUCCUGGG |
| | | CGGCAGCCUGGGCGGCGGCGGCAGCGGCAGCAACAUCAC |
| | | CAACCUCUGCCCCUUCGACGAGGUGUUCAACGCCACCAG |
| | | AUUCGCCAGCGUCUACGCCUGGAACCGGAAGCGGAUCAG |
| | | CAAUUGCGUGGCCGACUACAGCGUGCUGUACAACCUCGC |
| | | CCCUUUCUUCACCUUCAAGUGCUACGGCGUGUCCCCUAC |
| | | CAAGCUGAACGAUCUGUGCUUCACCAACGUGUACGCCGA |
| | | CAGCUUCGUGAUCAGGGGCGACGAAGUGCGACAGAUCG |
| | | CCCCAGGACAGACAGGAAACAUCGCCGAUUACAACUACA |
| | | AGCUGCCCGACGACUUCACCGGUUGCGUGAUCGCUUGGA |
| | | ACAGCAACAAGCUGGACAGCAAGGUCUCCGGCAACUACA |
| | | ACUACCGGUACCGGCUGUUCCGGAAGUCCAACCUGAAGC |
| | | CCUUCGAGCGGGACAUCAGCACAGAGAUCUACCAGGCCG |
| | | GCAACAAACCUUGUAACGGCGUGGCGGGCGUCAACUGC |
| | | UACUUCCCUCUGCGGAGCUACAGCUUCCGGCCUACAUAC |
| | | GGCGUGGGACACCAGCCUUACAGGGUGGUGGUGCUGAG |
| | | CUUCGAACUGCUGCACGCUCCAGCCACCGUCUGCGGACC |
| | | UAAAAAGAGCACCAACCUGGUGAAGAACAAGUGCGUGA |
| | | AUUUCUGAUGACGGCCGCAUACAGCAGCAAUUGGCAAG |
| | | CUGCUUACAUAGAACUCGCGGCGAUUGGCAUGCCGCCUU |
| | | AAAAUUUUUCUUUUCUUUUCCGAAUCGGAUUUUGUUUU |
| | | UAAUAUUUAAGCUUAAAAAAAAAAAAAAAAAAAAAAAA |
| | | AAAAAAAAAAAAAAAAAAAAAAAAAAAAAAAAAAAAAA |
| | | AAAAAAAAAAAAAAAAAAAAAAAAAAAAAAAAAAAAAA |
| | | AAAAAAAAAAAAAAAAAAAAAAAAA |
| Structure 4 (RNA | SEQ ID | GGAUAGGCGGCGCAUGAGAGAAGCCCAGACCAAUUACC |
| sequence) | NO.: 29 | UACCCAAAGCCACCAUGAAGGCCAACCUGCUGGUCCUGC |
| | | UGUGCGCCCUGGCCGCUGCCGACGCCGACACCAUCUGCA |
| | | UCGGCUACCACGCCAACAACAGCACCGACACCGUGGACA |
| | | CCGUGCUGGAGAAGAACGUGACCGUGACCCACAGCGUG |
| | | AACCUGCUGGAGGACAGCCACAACGGCAAGCUGUGCAG |
| | | ACUGAAGGGCAUCGCCCCCCUGCAGCUGGGCAAGUGCAA |
| | | CAUCGCCGGCUGGCUGCUGGGCAACCCCGAGUGCGACCC |
| | | CCUGCUGCCCGUGAGAAGCUGGAGCUACAUCGUGGAGA |
| | | CCCCCAACAGCGAGAACGGCAUCUGCUACCCCGGCGACU |
| | | UCAUCGACUACGAGGAGCUGAGAGAGCAGCUGAGCAGC |
| | | GUGAGCAGCUUCGAGAGAUUCGAGAUCUUCCCCAAGGA |
| | | GAGCAGCUGGCCCAACCACAACACCAACGGCGUGACCGC |
| | | CGCCUGCAGCCACGAGGGCAAGAGCAGCUUCUACAGAAA |
| | | CCUGCUGUGGCUGACCGAGAAGGAGGGCAGCUACCCCAA |
| | | GCUGAAGAACUCCUAUGUGAACAAGAAGGGCAAAGAGG |
| | | UGCUGGUGCUGUGGGGCAUCCACCACCCCCCCAACAGCA |
| | | AGGAGCAGCAGAACCUGUAUCAGAACGAGAACGCCUAC |
| | | GUGAGCGUGGUGACAAGCAACUACAACAGAAGAUUCAC |
| | | CCCCGAGAUCGCCGAGAGACCCAAGGUGAGAGACCAAGC |
| | | CGGCAGAAUGAACUACUACUGGACCCUGCUGAAGCCCGG |
| | | CGACACCAUCAUCUUCGAGGCCAACGGCAACCUGAUCGC |
| | | CCCUAUGUACGCCUUCGCCCUGAGCAGAGGCUUCGGCAG |
| | | CGGCAUCAUCACAAGCAACGCUAGCAUGCACGAGUGCAA |
| | | CACCAAGUGUCAGACCCCCCUGGGCGCCAUCAACAGCAG |
| | | CCUGCCCUAUCAGAACAUCCACCCCGUGACCAUCGGCGA |
| | | GUGCCCCAAGUACGUGAGAAGCGCCAAGCUGAGAAUGG |
| | | UGACCGGCCUGAGAAACAUCCCUAGCAUUCAGAGCAGA |
| | | GGCCUGUUCGGCGCCAUCGCCGGCUUCAUCGAGGGCGGC |
| | | UGGACCGGCAUGAUCGACGGCUGGUACGGCUACCACCAU |
| | | CAGAACGAGCAAGGCAGCGGCUACGCCGCCGAUCAAAAG |
| | | AGCACACAGAACGCCAUCAACGGCAUCACCAACAAGGUG |
| | | AACACCGUGAUCGAGAAGAUGAACAUUCAGUUCACCGC |
| | | CGUGGGCAAGGAGUUCAACAAGCUGGAGAAGAGAAUGG |
| | | AGAACCUCAACAAGAAGGUGGACGACGGCUUCCUGGAC |
| | | AUCUGGACCUACAACGCCGAGCUGCUGGUGCUGCUGGA |
| | | GAACGAGAGAACCCUGGACUUCCACGACAGCAACGUGA |
| | | AGAACCUGUACGAGAAGGUCAAGAGCCAACUGAAGAAC |
| | | AAUGCCAAGGAGAUCGGCAACGGCUGCUUCGAGUUCUA |
| | | CCACAAGUGCGACAACGAGUGCAUGGAGAGCGUGAGAA |
| | | ACGGCACCUACGACUACCCCAAGUACAGCGAGGAGAGCA |
| | | AGCUGAACAGAGAGAAGGUCGACGGCGUGAAGCUGGAG |
| | | AGCAUGGGCAUCUAUCAGAUCGGCGGCAGCGGCGCCAUC |
| | | GGCGGCUACAUCCCCGAGGCCCCCAGAGACGGCCAGGCC |
| | | UACGUGAGAAAGGACGGCGAGUGGGUGCUGCUGAGCAC |
| | | CUUCCUGGGCGGCAGCCUGGGCGGCGGCGGCAGCGGCAG |
| | | CAAGACAAUCAUCGCCCUGAGCCACAUCCUGUGCCUGGU |
| | | GUUCGCUCAGAAGCUGCCCGGCAACGACAACAGCACCGC |
| | | CACCCUGUGCCUGGGCCACCACGCCGUGCCCAACGGCAC |
| | | CAUCGUGAAAACAAUCACCAACGAUCAGAUCGAGGUGA |
| | | CCAACGCCACCGAGCUGGUGCAGAACAGCAGCAUCGGCG |
| | | AGAUCUGCGACAGCCCCCAUCAGAUCCUGGACGGCGAGA |
| | | ACUGCACCCUGAUCGACGCUCUGCUGGGCGAUCCUCAGU |
| | | GCGACGGCUUUCAGAACAAGAAGUGGGACCUGUUCGUG |
| | | GAGAGAAGCAAGGCCUACAGCAACUGCUACCCCUACGAU |
| | | GUCCCCGACUACGCUAGCCUGAGAAGCCUGGUGGCUAGC |
| | | AGCGGCACCCUGGAGUUCAACAACGAGAGCUUCAACUG |
| | | GACCGGCGUGACACAGAACGGCACAAGCAGCGCCUGCAU |
| | | CAGAAGAAGCAACAACAGCUUCUUCAGCAGACUGAACU |
| | | GGCUGACACAGCUGAACUUCAAGUACCCCGCCCUGAACG |
| | | UGACCAUGCCCAACAACGAGCAGUUCGACAAGCUGUACA |
| | | UCUGGGGCGUGCACCACCCCGUGACCGACAAGGAUCAGA |
| | | UCUUCCUGUACGCUCAGAGCAGCGGCAGAAUCACCGUGA |
| | | GCACCAAGAGAAGCCAACAAGCCGUGAUCCCCAACAUCG |
| | | GCUACAGACCUAGAAUCAGAAACAUCCCUAGCAGAAUC |
| | | AGCAUCUACUGGACCAUCGUGAAGCCCGGCGACAUCCUG |
| | | CUGAUCAACAGCACCGGCAACCUGAUCGCCCCUAGAGGC |
| | | UACUUCAAGAUCAGAAGCGGCAAGAGCAGCAUCAUGAG |
| | | AAGCGACGCCCCCAUCGGCAAGUGCAACAGCGAGUGCAU |
| | | CACCCCCAACGGCAGCAUCCCCAAUGAUAAGCCCUUUCA |
| | | GAACGUGAACAGAAUCACCUACGGCGCCUGCCCUAGAUA |
| | | CGUGAAGCAGAGCACCCUGAAGCUGGCCACCGGCAUGAG |
| | | AAACGUGCCCGAGAAGCAGACAAGAGGCAUCUUCGGCG |
| | | CCAUCGCCGGCUUCAUCGAGAACGGCUGGGAGGGCAUG |
| | | GUGGACGGCUGGUACGGCUUCAGACAUCAGAACAGCGA |
| | | GGGCAGAGGCCAAGCCGCCGACCUCAAGAGCACCCAAGC |
| | | CGCCAUCGAUCAGAUCAACGGCAAGCUGAACAGACUGA |
| | | UCGGCAAGACCAACGAGAAGUUCCAUCAGAUCGAGAAG |
| | | GAGUUCAGCGAGGUGGAGGGCAGAAUCCAAGACCUGGA |
| | | GAAGUACGUGGAGGACACCAAGAUCGACCUGUGGAGCU |
| | | ACAACGCCGAGCUGCUGGUGGCCCUGGAGAAUCAGCACA |
| | | CCAUCGACCUGACCGACAGCGAGAUGAACAAGCUGUUCG |
| | | AGAAGACCAAGAAGCAGCUGAGAGAGAACGCCGAGGAC |
| | | AUGGGCAACGGCUGCUUCAAGAUCUACCACAAGUGCGA |
| | | CAACGCCUGCAUCGGCAGCAUCAGAAACGGCACCUACGA |
| | | CCACGACGUGUACAGAGACGAGGCCCUGAACAACAGAU |
| | | UUCAGAUCAAGGGCGUGUGAUGAUGACGGCCGCAUACA |
| | | GCAGCAAUUGGCAAGCUGCUUACAUAGAACUCGCGGCG |
| | | AUUGGCAUGCCGCCUUAAAAUUUUUCUUUUCUUUUCCG |
| | | AAUCGGAUUUUGUUUUUAAUAUUUAAGCUUAAAAAAAA |
| | | AAAAAAAAAAAAAAAAAAAAAAAAAAAAAAAAAAAAAA |
| | | AAAAAAAAAAAAAAAAAAAAAAAAAAAAAAAAAAAAAA |
| | | AAAAAAAAAAAAAAAAAAAAAAAAAAAAAAAAAAAAAA |
| | | AAA |
| Structure 5 (RNA | SEQ ID | GGAUAGGCGGCGCAUGAGAGAAGCCCAGACCAAUUACC |
| sequence) | NO.: 30 | UACCCAAAGCCACCAUGAAGGCCAACCUGCUGGUCCUGC |
| | | UGUGCGCCCUGGCCGCUGCCGACGCCGACACCAUCUGCA |
| | | UCGGCUACCACGCCAACAACAGCACCGACACCGUGGACA |
| | | CCGUGCUGGAGAAGAACGUGACCGUGACCCACAGCGUG |
| | | AACCUGCUGGAGGACAGCCACAACGGCAAGCUGUGCAG |
| | | ACUGAAGGGCAUCGCCCCCCUGCAGCUGGGCAAGUGCAA |
| | | CAUCGCCGGCUGGCUGCUGGGCAACCCCGAGUGCGACCC |
| | | CCUGCUGCCCGUGAGAAGCUGGAGCUACAUCGUGGAGA |
| | | CCCCCAACAGCGAGAACGGCAUCUGCUACCCCGGCGACU |
| | | UCAUCGACUACGAGGAGCUGAGAGAGCAGCUGAGCAGC |
| | | GUGAGCAGCUUCGAGAGAUUCGAGAUCUUCCCCAAGGA |
| | | GAGCAGCUGGCCCAACCACAACACCAACGGCGUGACCGC |
| | | CGCCUGCAGCCACGAGGGCAAGAGCAGCUUCUACAGAAA |
| | | CCUGCUGUGGCUGACCGAGAAGGAGGGCAGCUACCCCAA |
| | | GCUGAAGAACUCCUAUGUGAACAAGAAGGGCAAAGAGG |
| | | UGCUGGUGCUGUGGGGCAUCCACCACCCCCCCAACAGCA |
| | | AGGAGCAGCAGAACCUGUAUCAGAACGAGAACGCCUAC |
| | | GUGAGCGUGGUGACAAGCAACUACAACAGAAGAUUCAC |
| | | CCCCGAGAUCGCCGAGAGACCCAAGGUGAGAGACCAAGC |
| | | CGGCAGAAUGAACUACUACUGGACCCUGCUGAAGCCCGG |
| | | CGACACCAUCAUCUUCGAGGCCAACGGCAACCUGAUCGC |
| | | CCCUAUGUACGCCUUCGCCCUGAGCAGAGGCUUCGGCAG |
| | | CGGCAUCAUCACAAGCAACGCUAGCAUGCACGAGUGCAA |
| | | CACCAAGUGUCAGACCCCCCUGGGCGCCAUCAACAGCAG |
| | | CCUGCCCUAUCAGAACAUCCACCCCGUGACCAUCGGCGA |
| | | GUGCCCCAAGUACGUGAGAAGCGCCAAGCUGAGAAUGG |
| | | UGACCGGCCUGAGAAACAUCCCUAGCAUUCAGAGCAGA |
| | | GGCCUGUUCGGCGCCAUCGCCGGCUUCAUCGAGGGCGGC |
| | | UGGACCGGCAUGAUCGACGGCUGGUACGGCUACCACCAU |
| | | CAGAACGAGCAAGGCAGCGGCUACGCCGCCGAUCAAAAG |
| | | AGCACACAGAACGCCAUCAACGGCAUCACCAACAAGGUG |
| | | AACACCGUGAUCGAGAAGAUGAACAUUCAGUUCACCGC |
| | | CGUGGGCAAGGAGUUCAACAAGCUGGAGAAGAGAAUGG |
| | | AGAACCUCAACAAGAAGGUGGACGACGGCUUCCUGGAC |
| | | AUCUGGACCUACAACGCCGAGCUGCUGGUGCUGCUGGA |
| | | GAACGAGAGAACCCUGGACUUCCACGACAGCAACGUGA |
| | | AGAACCUGUACGAGAAGGUCAAGAGCCAACUGAAGAAC |
| | | AAUGCCAAGGAGAUCGGCAACGGCUGCUUCGAGUUCUA |
| | | CCACAAGUGCGACAACGAGUGCAUGGAGAGCGUGAGAA |
| | | ACGGCACCUACGACUACCCCAAGUACAGCGAGGAGAGCA |
| | | AGCUGAACAGAGAGAAGGUCGACGGCGUGAAGCUGGAG |
| | | AGCAUGGGCAUCUAUCAGAUCUGAUGACGGCCGCAUAC |
| | | AGCAGCAAUUGGCAAGCUGCUUACAUAGAACUCGCGGC |
| | | GAUUGGCAUGCCGCCUUAAAAUUUUUCUUUUCUUUUCC |
| | | GAAUCGGAUUUUGUUUUUAAUAUUUAAGCUUAAAAAAA |
| | | AAAAAAAAAAAAAAAAAAAAAAAAAAAAAAAAAAAAAA |
| | | AAAAAAAAAAAAAAAAAAAAAAAAAAAAAAAAAAAAAA |
| | | AAAAAAAAAAAAAAAAAAAAAAAAAAAAAAAAAAAAAA |
| | | AAAA |
| Structure 6 (RNA | SEQ ID | GGAUAGGCGGCGCAUGAGAGAAGCCCAGACCAAUUACC |
| sequence) | NO.: 31 | UACCCAAAGCCACCAUGAAGACAAUCAUCGCCCUGAGCC |
| | | ACAUCCUGUGCCUGGUGUUCGCUCAGAAGCUGCCCGGCA |
| | | ACGACAACAGCACCGCCACCCUGUGCCUGGGCCACCACG |
| | | CCGUGCCCAACGGCACCAUCGUGAAAACAAUCACCAACG |
| | | AUCAGAUCGAGGUGACCAACGCCACCGAGCUGGUGCAG |
| | | AACAGCAGCAUCGGCGAGAUCUGCGACAGCCCCCAUCAG |
| | | AUCCUGGACGGCGAGAACUGCACCCUGAUCGACGCUCUG |
| | | CUGGGCGAUCCUCAGUGCGACGGCUUUCAGAACAAGAA |
| | | GUGGGACCUGUUCGUGGAGAGAAGCAAGGCCUACAGCA |
| | | ACUGCUACCCCUACGAUGUCCCCGACUACGCUAGCCUGA |
| | | GAAGCCUGGUGGCUAGCAGCGGCACCCUGGAGUUCAAC |
| | | AACGAGAGCUUCAACUGGACCGGCGUGACACAGAACGG |
| | | CACAAGCAGCGCCUGCAUCAGAAGAAGCAACAACAGCUU |
| | | CUUCAGCAGACUGAACUGGCUGACACAGCUGAACUUCA |
| | | AGUACCCCGCCCUGAACGUGACCAUGCCCAACAACGAGC |
| | | AGUUCGACAAGCUGUACAUCUGGGGCGUGCACCACCCCG |
| | | UGACCGACAAGGAUCAGAUCUUCCUGUACGCUCAGAGC |
| | | AGCGGCAGAAUCACCGUGAGCACCAAGAGAAGCCAACA |
| | | AGCCGUGAUCCCCAACAUCGGCUACAGACCUAGAAUCAG |
| | | AAACAUCCCUAGCAGAAUCAGCAUCUACUGGACCAUCGU |
| | | GAAGCCCGGCGACAUCCUGCUGAUCAACAGCACCGGCAA |
| | | CCUGAUCGCCCCUAGAGGCUACUUCAAGAUCAGAAGCGG |
| | | CAAGAGCAGCAUCAUGAGAAGCGACGCCCCCAUCGGCAA |
| | | GUGCAACAGCGAGUGCAUCACCCCCAACGGCAGCAUCCC |
| | | CAAUGAUAAGCCCUUUCAGAACGUGAACAGAAUCACCU |
| | | ACGGCGCCUGCCCUAGAUACGUGAAGCAGAGCACCCUGA |
| | | AGCUGGCCACCGGCAUGAGAAACGUGCCCGAGAAGCAG |
| | | ACAAGAGGCAUCUUCGGCGCCAUCGCCGGCUUCAUCGAG |
| | | AACGGCUGGGAGGGCAUGGUGGACGGCUGGUACGGCUU |
| | | CAGACAUCAGAACAGCGAGGGCAGAGGCCAAGCCGCCGA |
| | | CCUCAAGAGCACCCAAGCCGCCAUCGAUCAGAUCAACGG |
| | | CAAGCUGAACAGACUGAUCGGCAAGACCAACGAGAAGU |
| | | UCCAUCAGAUCGAGAAGGAGUUCAGCGAGGUGGAGGGC |
| | | AGAAUCCAAGACCUGGAGAAGUACGUGGAGGACACCAA |
| | | GAUCGACCUGUGGAGCUACAACGCCGAGCUGCUGGUGG |
| | | CCCUGGAGAAUCAGCACACCAUCGACCUGACCGACAGCG |
| | | AGAUGAACAAGCUGUUCGAGAAGACCAAGAAGCAGCUG |
| | | AGAGAGAACGCCGAGGACAUGGGCAACGGCUGCUUCAA |
| | | GAUCUACCACAAGUGCGACAACGCCUGCAUCGGCAGCAU |
| | | CAGAAACGGCACCUACGACCACGACGUGUACAGAGACGA |
| | | GGCCCUGAACAACAGAUUUCAGAUCAAGGGCGUGUGAU |
| | | GACGGCCGCAUACAGCAGCAAUUGGCAAGCUGCUUACA |
| | | UAGAACUCGCGGCGAUUGGCAUGCCGCCUUAAAAUUUU |
| | | UCUUUUCUUUUCCGAAUCGGAUUUUGUUUUUAAUAUUU |
| | | AAGCUUAAAAAAAAAAAAAAAAAAAAAAAAAAAAAAAA |
| | | AAAAAAAAAAAAAAAAAAAAAAAAAAAAAAAAAAAAAA |
| | | AAAAAAAAAAAAAAAAAAAAAAAAAAAAAAAAAAAAAA |
| | | AAAAAAAAAAAAAAAAA |

### INCORPORATION BY REFERENCE

The entire disclosure of each of the patent documents and scientific articles cited herein are incorporated by reference for all purposes.

### EQUIVALENTS

The disclosure may be embodied in other specific forms without departing from the spirit or essential characteristics thereof. The foregoing embodiments are therefore to be considered in all respects illustrative rather than limiting the disclosure described herein. Scope of the disclosure is thus indicated by the appended claims rather than by the foregoing description, and all changes that come within the meaning and range of equivalency of the claims are intended to be embraced therein.

1. A purified ribonucleic acid (RNA) molecule comprising:
a) a first nucleotide sequence encoding a first antigen, or an antigenic fragment thereof, from a first virus variant;
b) a second nucleotide sequence encoding a multimerization domain; and
c) a third nucleotide sequence encoding a second antigen, or an antigenic fragment thereof, from a second virus variant,
wherein the first virus variant and the second virus variant are different, and
wherein the first nucleotide sequence, the second nucleotide sequence, and the third nucleotide sequence are operably linked to each other in a 5′-to-3′ direction.

2. The purified RNA of claim 1, wherein the RNA is a messenger RNA (mRNA), a self-replicating RNA, or is not a self-replicating RNA.

3.-4. (canceled)

5. The purified RNA of claim 1, wherein the virus is selected from the group consisting of:
(i) a Influenza virus, Rabies virus, respiratory syncytial virus (RSV), and coronavirus; or
(ii) 229E (alpha coronavirus), NL63 (alpha coronavirus), OC43 (beta coronavirus), HKU1 (beta coronavirus), MERS-CoV (MERS), SARS-CoV (SARS), and SARS-CoV-2 (COVID-19.

6.-7. (canceled)

8. The purified RNA of claim 5, wherein the first virus variant or the second virus variant is selected from the group consisting of:
(i) Wuhan-Hu-1, Alpha, Beta, Gamma, Delta, Epsilon Eta, Iota, Kappa, 1.617.3, Mu, Zeta, and Omicron;
(ii) Wuhan-Hu-1 and Delta;
(ii) the influenza A virus is selected from the group consisting of H1N1 (PR8), H2N2, H1N2, H3N2 (Udorn), N2-NA, Wyo03, PC73, H5N1, H5N2, H5N8, H5N9, H7N2, H7N3, H7N7, H9N2, H10N7, and H10N3; or
(iv) H1N1 (PR8) and H3N2 (Udorn).

9. (canceled)

10. The purified RNA of claim 8, wherein the first antigen, or the antigenic fragment thereof, and/or the second antigen, or the antigenic fragment thereof, comprises:
i) a viral envelope protein, viral spike protein, viral membrane protein, or viral capsid protein;
ii) a receptor-binding domain (RBD);
iii) an RNA polymerase subunit, hemagglutinin (HA), nucleoprotein (NP), neuraminidase (NA), matrix protein 1 (M1), matrix protein 2 (M2), non-structural protein NS1, or non-structural protein NEP; or
iv) comprises a hemagglutinin (HA) protein.

11.-21. (canceled)

22. The purified RNA of claim 1, wherein the multimerization domain:
i) is selected from the group consisting of a dimerization domain, trimerization domain, and a tetramerization domain;
ii) is selected from the group consisting of enterobacteria phage T4, GCN4pII, GCN4-pLI, and p53;
(iii) comprises a leucine zipper or a fibritin foldon domain; or
(iv) comprises a trimerization domain.

23-27. (canceled)

28. The purified RNA molecule of claim 1, further comprising a sequence encoding a first linker connecting the first antigen, or the antigenic fragment thereof, to the multimerization domain; and/or further comprising a sequence encoding a second linker connecting the second antigen, or the antigenic fragment thereof, to the multimerization domain.

29.-31. (canceled)

32. The purified RNA molecule of claim 28, wherein the first linker and/or the second linker encodes the amino acid sequence:
i) selected from the group consisting of (GS)n (SEQ ID NO: 9), (G2S)n (SEQ ID NO: 10), (G3S)n (SEQ ID NO: 11), (G4S)n (SEQ ID NO: 32), and (G)n (SEQ ID NO: 33), and wherein n is an integer from 2 to 20;
ii) (GGSGGD)n (SEQ ID NO: 34) or (GGSGGE)n (SEQ ID NO: 35), and wherein n is an integer from 2 to 6;
iii) selected from the group consisting of (GGGSGSGGGGS)n (SEQ ID NO: 36) and (GGGGGPGGGGP)n (SEQ ID NO: 37), and wherein n is an integer from 1 to 3;
iv) selected from the group consisting of (GX)n, (GGX)n, (GGGX)n, (GGGGX)n, and (GzX)n, wherein z is between 1 and 20, and wherein n is at least 8;
wherein X is serine, aspartic acid, glutamic acid, threonine, or proline; or
v) GGSG (SEQ ID NO: 38) or GGSLGGGGSGS (SEQ ID NO: 39).

33.-46. (canceled)

47. The purified RNA of claim 1, wherein the first antigen, or the antigenic fragment thereof, and/or the second antigen, or the antigenic fragment thereof, is:
i) encoded by the nucleotide sequence having at least 90% sequence identity to the sequence of any one of SEQ ID NOs: 3-4, 7, and 8; or
ii) comprises a sequence having at least 90% sequence identity to the sequence of any one of SEQ ID NOs: 19, 20, 22, and 23.

48.-50. (canceled)

51. The purified RNA of claim 1, wherein the purified RNA further comprises:
i) a m7GpppNm-, where Nm denotes any nucleotide with a 2′ O methylation (Cap 1) 5′ to the first nucleotide sequence;
ii) a 5′ UTR 3′ to a Cap 1 and 5′ to the first nucleotide sequence;
iii) a sequence encoding a signal peptide 3′ to the 5′ UTR and 5′ to the first nucleotide sequence; and/or
iv) a sequence encoding a 3′ UTR 3′ to the second nucleotide sequence.

52.-54. (canceled)

55. A purified RNA molecule comprising a sequence having at least 90% sequence identity to the sequence of any one of SEQ ID NOs: 26-31.

56.-61. (canceled)

62. A purified RNA molecule comprising, from 5′ to 3′:
i) a) a m7GpppNm-, where Nm denotes any nucleotide with a 2′ O methylation (Cap 1);
b) a 5′ UTR;
c) a first nucleotide sequence encoding a signal peptide;
d) a second nucleotide sequence encoding a first antigen, or an antigenic fragment thereof, from a wild-type SARS-CoV-2 virus or a first virus variant selected from the group consisting of: a Wuhan-Hu-1 variant, an Omicron variant, and a Delta variant;
e) a third nucleotide sequence encoding a first linker;
f) a fourth nucleotide sequence encoding a T4 foldon domain;
g) a fifth nucleotide sequence encoding a second linker;
h) a sixth nucleotide sequence encoding a second antigen, or an antigenic fragment thereof, from a wild-type SARS-CoV-2 virus or a second virus variant selected from the group consisting of: a Wuhan-Hu-1 variant, an Omicron variant, and a Delta variant;
i) a 3′ UTR; and
j) a poly A tail;
ii) a) a m7GpppNm-, where Nm denotes any nucleotide with a 2′ O methylation (Cap 1);
b) a 5′ UTR;
c) a first nucleotide sequence encoding a signal peptide;
d) a second nucleotide sequence encoding a first antigen, or an antigenic fragment thereof, from an Omicron variant;
e) a third nucleotide sequence encoding a first linker;
f) a fourth nucleotide sequence encoding a T4 foldon domain;
g) a fifth nucleotide sequence encoding a second linker;
h) a sixth nucleotide sequence encoding a second antigen, or an antigenic fragment thereof, from a wild-type SARS-CoV-2 virus;
i) a 3′ UTR; and
i) a poly A tail;
iii) a) a m7GpppNm-, where Nm denotes any nucleotide with a 2′ O methylation (Cap 1);
b) a 5′ UTR;
c) a first nucleotide sequence encoding a signal peptide;
d) a second nucleotide sequence encoding a first antigen, or an antigenic fragment thereof, from a wild-type SARS-CoV-2 virus;
e) a third nucleotide sequence encoding a first linker;
f) a fourth nucleotide sequence encoding a T4 foldon domain;
g) a fifth nucleotide sequence encoding a second linker;
h) a sixth nucleotide sequence encoding a second antigen, or an antigenic fragment thereof, from an Omicron variant;
i) a 3′ UTR; and
j) a poly A tail;
iv) a) a m7GpppNm-, where Nm denotes any nucleotide with a 2′ O methylation (Cap 1);
b) a 5′ UTR;
c) a first nucleotide sequence encoding a first antigen, or an antigenic fragment thereof, from an influenza virus selected from the group consisting of H1N1 (PR8), H2N2, H1N2, H3N2 (Udorn), N2-NA, Wyo03, PC73, H5N1, H5N2, H5N8, H5N9, H7N2, H7N3, H7N7, H9N2, H10N7, and H10N3;
d) optionally, a second nucleotide sequence encoding a first linker;
e) optionally, a third nucleotide sequence encoding a T4 foldon domain;
f) optionally, a fourth nucleotide sequence encoding a second linker;
g) optionally, a fifth nucleotide sequence encoding a second antigen, or an antigenic fragment thereof, from an influenza virus selected from the group consisting of H1N1 (PR8), H2N2, H1N2, H3N2 (Udorn), N2-NA, Wyo03, PC73, H5N1, H5N2, H5N8, H5N9, H7N2, H7N3, H7N7, H9N2, H10N7, and H10N3;
h) a 3′ UTR; and
i) a poly A tail;
v) a) a m7GpppNm-, where Nm denotes any nucleotide with a 2′ O methylation (Cap 1);
b) a 5′ UTR;
c) a first nucleotide sequence encoding a first antigen, or an antigenic fragment thereof, from a H1N1 (PR8) influenza virus;
d) a second nucleotide sequence encoding a first linker;
e) a third nucleotide sequence encoding a T4 foldon domain;
f) a fourth nucleotide sequence encoding a second linker;
g) a fifth nucleotide sequence encoding a second antigen, or an antigenic fragment thereof, from a H3N2 (Udorn) influenza virus;
h) a 3′ UTR; and
i) a poly A tail;
vi) a) a m7GpppNm-, where Nm denotes any nucleotide with a 2′ O methylation (Cap 1);
b) a 5′ UTR;
c) a nucleotide sequence encoding a first antigen, or an antigenic fragment thereof, from a H1N1 (PR8) influenza virus;
d) a 3′ UTR; and
e) a poly A tail; or
vii) a) a m7GpppNm-, where Nm denotes any nucleotide with a 2′ O methylation (Cap 1);
b) a 5′ UTR;
c) a nucleotide sequence encoding a first antigen, or an antigenic fragment thereof, from a H3N2 (Udorn) influenza virus;
d) a 3′ UTR; and
e) a poly A tail.

63.-68. (canceled)

69. An isolated polypeptide encoded by the purified RNA molecule of claim 1.

70. A composition comprising the purified RNA of claim 1 and a delivery vehicle.

71. The composition of claim 70, wherein the delivery vehicle comprises a lipid nanoparticle (LNP).

72.-73. (canceled)

74. The composition of claim 70, wherein the composition comprises a particle size of:
i) no more than about 300 nanometers (nm);
ii) about 50 to about to 300 nm;
iii) no more than about 150 nm; or
iv) no more than about 150 nm.

75.-81. (canceled)

82. A kit comprising the purified RNA of claim 1, a delivery vehicle, and instructions for use.

83. A method of treating or preventing disease in a subject comprising administering to the subject an effective amount of the composition of claim 70.

84. The method of claim 83, wherein the disease is caused by a virus is selected from the group consisting of a Influenza virus, Rabies virus, respiratory syncytial virus (RSV) and coronavirus.

85. (canceled)

86. The method of claim 84, wherein
i) the coronavirus is selected from the group consisting of 229E (alpha coronavirus), NL63 (alpha coronavirus), OC43 (beta coronavirus), HKU1 (beta coronavirus), MERS-CoV (MERS), SARS-CoV (SARS), and SARS-CoV-2 (COVID-19); or
ii) the influenza A virus is selected from the group consisting of H1N1 (PR8), H2N2, H1N2, H3N2 (Udorn), N2-NA, Wyo03, PC73, H5N1, H5N2, H5N8, H5N9, H7N2, H7N3, H7N7, H9N2, H10N7, and H10N3.

87.-92. (canceled)

93. A method for stimulating an immune response in a subject comprising administering to the subject an effective amount of the composition of claim 70.

94. A method for preparing a RNA-LNP composition comprising:
a) mixing an ethanol phase comprising one or more lipids and an aqueous phase comprising the purified RNA molecule of claim 1; and
b) purifying the RNA-LNP generated from step a).

95.-101. (canceled)


SEQUENCE LISTING (40 sequences)

SEQ ID NO: 1 - DNA, 89 bp, synthetic construct
  source 1..89: mol_type = other DNA
gccatcggcggctacatccccgaggcccccagagacggccaggcctacgtgagaaaggacggcgagtgggtgctgctgagcaccttcct

SEQ ID NO: 2 - DNA, 72 bp, synthetic construct
  source 1..72: mol_type = other DNA
atgcctatgggcagcctgcagcccctggccaccctgtacctgctgggcatgctggtggccagcgtgctggcc

SEQ ID NO: 3 - DNA, 633 bp, synthetic construct
  source 1..633: mol_type = other DNA
aacatcaccaacctctgccccttcgacgaggtgttcaacgccaccagattcgccagcgtctacgcctggaaccggaagcggatcagcaattgcgtggccgactacagcgtgctgtacaacctcgcccctttcttcaccttcaagtgctacggcgtgtcccctaccaagctgaacgatctgtgcttcaccaacgtgtacgccgacagcttcgtgatcaggggcgacgaagtgcgacagatcgccccaggacagacaggaaacatcgccgattacaactacaagctgcccgacgacttcaccggttgcgtgatcgcttggaacagcaacaagctggacagcaaggtctccggcaactacaactaccggtaccggctgttccggaagtccaacctgaagcccttcgagcgggacatcagcacagagatctaccaggccggcaacaaaccttgtaacggcgtggcgggcgtcaactgctacttccctctgcggagctacagcttccggcctacatacggcgtgggacaccagccttacagggtggtggtgctgagcttcgaactgctgcacgctccagccaccgtctgcggacctaaaaagagcaccaacctggtgaagaacaagtgcgtgaatttc

SEQ ID NO: 4 - DNA, 633 bp, synthetic construct
  source 1..633: mol_type = other DNA
aacatcaccaacctgtgccccttcggcgaggtgttcaacgccaccagattcgccagcgtgtacgcctggaacagaaagagaatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcagaggcgacgaggtgagacagatcgcccccggccagaccggcaagatcgccgactacaactacaagctgcccgacgacttcaccggctgcgtgatcgcctggaacagcaacaacctggacagcaaggtgggcggcaactacaactacctgtacagactgttcagaaagagcaacctgaagcccttcgagagagacatcagcaccgagatctaccaggccggcagcaccccctgcaacggcgtggagggcttcaactgctacttccccctgcagagctacggcttccagcccaccaacggcgtgggctaccagccctacagagtggtggtgctgagcttcgagctgctgcacgcccccgccaccgtgtgcggccccaagaagtccaccaacctggtgaagaacaagtgcgtgaacttc

SEQ ID NO: 5 - DNA, 12 bp, synthetic construct
  source 1..12: mol_type = other DNA
ggcggcagcggc

SEQ ID NO: 6 - DNA, 33 bp, synthetic construct
  source 1..33: mol_type = other DNA
ggcggcagcctgggcggcggcggcagcggcagc

SEQ ID NO: 7 - DNA, 1587 bp, synthetic construct
  source 1..1587: mol_type = other DNA
atgaaggccaacctgctggtcctgctgtgcgccctggccgctgccgacgccgacaccatctgcatcggctaccacgccaacaacagcaccgacaccgtggacaccgtgctggagaagaacgtgaccgtgacccacagcgtgaacctgctggaggacagccacaacggcaagctgtgcagactgaagggcatcgcccccctgcagctgggcaagtgcaacatcgccggctggctgctgggcaaccccgagtgcgaccccctgctgcccgtgagaagctggagctacatcgtggagacccccaacagcgagaacggcatctgctaccccggcgacttcatcgactacgaggagctgagagagcagctgagcagcgtgagcagcttcgagagattcgagatcttccccaaggagagcagctggcccaaccacaacaccaacggcgtgaccgccgcctgcagccacgagggcaagagcagcttctacagaaacctgctgtggctgaccgagaaggagggcagctaccccaagctgaagaactcctatgtgaacaagaagggcaaagaggtgctggtgctgtggggcatccaccacccccccaacagcaaggagcagcagaacctgtatcagaacgagaacgcctacgtgagcgtggtgacaagcaactacaacagaagattcacccccgagatcgccgagagacccaaggtgagagaccaagccggcagaatgaactactactggaccctgctgaagcccggcgacaccatcatcttcgaggccaacggcaacctgatcgcccctatgtacgccttcgccctgagcagaggcttcggcagcggcatcatcacaagcaacgctagcatgcacgagtgcaacaccaagtgtcagacccccctgggcgccatcaacagcagcctgccctatcagaacatccaccccgtgaccatcggcgagtgccccaagtacgtgagaagcgccaagctgagaatggtgaccggcctgagaaacatccctagcattcagagcagaggcctgttcggcgccatcgccggcttcatcgagggcggctggaccggcatgatcgacggctggtacggctaccaccatcagaacgagcaaggcagcggctacgccgccgatcaaaagagcacacagaacgccatcaacggcatcaccaacaaggtgaacaccgtgatcgagaagatgaacattcagttcaccgccgtgggcaaggagttcaacaagctggagaagagaatggagaacctcaacaagaaggtggacgacggcttcctggacatctggacctacaacgccgagctgctggtgctgctggagaacgagagaaccctggacttccacgacagcaacgtgaagaacctgtacgagaaggtcaagagccaactgaagaacaatgccaaggagatcggcaacggctgcttcgagttctaccacaagtgcgacaacgagtgcatggagagcgtgagaaacggcacctacgactaccccaagtacagcgaggagagcaagctgaacagagagaaggtcgacggcgtgaagctggagagcatgggcatctatcagatc

SEQ ID NO: 8 - DNA, 1563 bp, synthetic construct
  source 1..1563: mol_type = other DNA
aagacaatcatcgccctgagccacatcctgtgcctggtgttcgctcagaagctgcccggcaacgacaacagcaccgccaccctgtgcctgggccaccacgccgtgcccaacggcaccatcgtgaaaacaatcaccaacgatcagatcgaggtgaccaacgccaccgagctggtgcagaacagcagcatcggcgagatctgcgacagcccccatcagatcctggacggcgagaactgcaccctgatcgacgctctgctgggcgatcctcagtgcgacggctttcagaacaagaagtgggacctgttcgtggagagaagcaaggcctacagcaactgctacccctacgatgtccccgactacgctagcctgagaagcctggtggctagcagcggcaccctggagttcaacaacgagagcttcaactggaccggcgtgacacagaacggcacaagcagcgcctgcatcagaagaagcaacaacagcttcttcagcagactgaactggctgacacagctgaacttcaagtaccccgccctgaacgtgaccatgcccaacaacgagcagttcgacaagctgtacatctggggcgtgcaccaccccgtgaccgacaaggatcagatcttcctgtacgctcagagcagcggcagaatcaccgtgagcaccaagagaagccaacaagccgtgatccccaacatcggctacagacctagaatcagaaacatccctagcagaatcagcatctactggaccatcgtgaagcccggcgacatcctgctgatcaacagcaccggcaacctgatcgcccctagaggctacttcaagatcagaagcggcaagagcagcatcatgagaagcgacgcccccatcggcaagtgcaacagcgagtgcatcacccccaacggcagcatccccaatgataagccctttcagaacgtgaacagaatcacctacggcgcctgccctagatacgtgaagcagagcaccctgaagctggccaccggcatgagaaacgtgcccgagaagcagacaagaggcatcttcggcgccatcgccggcttcatcgagaacggctgggagggcatggtggacggctggtacggcttcagacatcagaacagcgagggcagaggccaagccgccgacctcaagagcacccaagccgccatcgatcagatcaacggcaagctgaacagactgatcggcaagaccaacgagaagttccatcagatcgagaaggagttcagcgaggtggagggcagaatccaagacctggagaagtacgtggaggacaccaagatcgacctgtggagctacaacgccgagctgctggtggccctggagaatcagcacaccatcgacctgaccgacagcgagatgaacaagctgttcgagaagaccaagaagcagctgagagagaacgccgaggacatgggcaacggctgcttcaagatctaccacaagtgcgacaacgcctgcatcggcagcatcagaaacggcacctacgaccacgacgtgtacagagacgaggccctgaacaacagatttcagatcaagggcgtgtga

SEQ ID NO: 9 - protein, 40 aa, synthetic construct
  VARIANT 5..40: note = GS repeats may be deleted
GSGSGSGSGSGSGSGSGSGSGSGSGSGSGSGSGSGSGSGS

SEQ ID NO: 10 - protein, 60 aa, synthetic construct
  VARIANT 7..60: note = GGS repeats may be deleted
GGSGGSGGSGGSGGSGGSGGSGGSGGSGGSGGSGGSGGSGGSGGSGGSGGSGGSGGSGGS

SEQ ID NO: 11 - protein, 80 aa, synthetic construct
  VARIANT 9..80: note = GGGS repeats may be deleted
GGGSGGGSGGGSGGGSGGGSGGGSGGGSGGGSGGGSGGGSGGGSGGGSGGGSGGGSGGGSGGGSGGGSGGGSGGGSGGGS

SEQ ID NO: 12 - DNA, 3285 bp, synthetic construct
  source 1..3285: mol_type = other DNA
atgaaggccaacctgctggtcctgctgtgcgccctggccgctgccgacgccgacaccatctgcatcggctaccacgccaacaacagcaccgacaccgtggacaccgtgctggagaagaacgtgaccgtgacccacagcgtgaacctgctggaggacagccacaacggcaagctgtgcagactgaagggcatcgcccccctgcagctgggcaagtgcaacatcgccggctggctgctgggcaaccccgagtgcgaccccctgctgcccgtgagaagctggagctacatcgtggagacccccaacagcgagaacggcatctgctaccccggcgacttcatcgactacgaggagctgagagagcagctgagcagcgtgagcagcttcgagagattcgagatcttccccaaggagagcagctggcccaaccacaacaccaacggcgtgaccgccgcctgcagccacgagggcaagagcagcttctacagaaacctgctgtggctgaccgagaaggagggcagctaccccaagctgaagaactcctatgtgaacaagaagggcaaagaggtgctggtgctgtggggcatccaccacccccccaacagcaaggagcagcagaacctgtatcagaacgagaacgcctacgtgagcgtggtgacaagcaactacaacagaagattcacccccgagatcgccgagagacccaaggtgagagaccaagccggcagaatgaactactactggaccctgctgaagcccggcgacaccatcatcttcgaggccaacggcaacctgatcgcccctatgtacgccttcgccctgagcagaggcttcggcagcggcatcatcacaagcaacgctagcatgcacgagtgcaacaccaagtgtcagacccccctgggcgccatcaacagcagcctgccctatcagaacatccaccccgtgaccatcggcgagtgccccaagtacgtgagaagcgccaagctgagaatggtgaccggcctgagaaacatccctagcattcagagcagaggcctgttcggcgccatcgccggcttcatcgagggcggctggaccggcatgatcgacggctggtacggctaccaccatcagaacgagcaaggcagcggctacgccgccgatcaaaagagcacacagaacgccatcaacggcatcaccaacaaggtgaacaccgtgatcgagaagatgaacattcagttcaccgccgtgggcaaggagttcaacaagctggagaagagaatggagaacctcaacaagaaggtggacgacggcttcctggacatctggacctacaacgccgagctgctggtgctgctggagaacgagagaaccctggacttccacgacagcaacgtgaagaacctgtacgagaaggtcaagagccaactgaagaacaatgccaaggagatcggcaacggctgcttcgagttctaccacaagtgcgacaacgagtgcatggagagcgtgagaaacggcacctacgactaccccaagtacagcgaggagagcaagctgaacagagagaaggtcgacggcgtgaagctggagagcatgggcatctatcagatcggcggcagcggcgccatcggcggctacatccccgaggcccccagagacggccaggcctacgtgagaaaggacggcgagtgggtgctgctgagcaccttcctgggcggcagcctgggcggcggcggcagcggcagcaagacaatcatcgccctgagccacatcctgtgcctggtgttcgctcagaagctgcccggcaacgacaacagcaccgccaccctgtgcctgggccaccacgccgtgcccaacggcaccatcgtgaaaacaatcaccaacgatcagatcgaggtgaccaacgccaccgagctggtgcagaacagcagcatcggcgagatctgcgacagcccccatcagatcctggacggcgagaactgcaccctgatcgacgctctgctgggcgatcctcagtgcgacggctttcagaacaagaagtgggacctgttcgtggagagaagcaaggcctacagcaactgctacccctacgatgtccccgactacgctagcctgagaagcctggtggctagcagcggcaccctggagttcaacaacgagagcttcaactggaccggcgtgacacagaacggcacaagcagcgcctgcatcagaagaagcaacaacagcttcttcagcagactgaactggctgacacagctgaacttcaagtaccccgccctgaacgtgaccatgcccaacaacgagcagttcgacaagctgtacatctggggcgtgcaccaccccgtgaccgacaaggatcagatcttcctgtacgctcagagcagcggcagaatcaccgtgagcaccaagagaagccaacaagccgtgatccccaacatcggctacagacctagaatcagaaacatccctagcagaatcagcatctactggaccatcgtgaagcccggcgacatcctgctgatcaacagcaccggcaacctgatcgcccctagaggctacttcaagatcagaagcggcaagagcagcatcatgagaagcgacgcccccatcggcaagtgcaacagcgagtgcatcacccccaacggcagcatccccaatgataagccctttcagaacgtgaacagaatcacctacggcgcctgccctagatacgtgaagcagagcaccctgaagctggccaccggcatgagaaacgtgcccgagaagcagacaagaggcatcttcggcgccatcgccggcttcatcgagaacggctgggagggcatggtggacggctggtacggcttcagacatcagaacagcgagggcagaggccaagccgccgacctcaagagcacccaagccgccatcgatcagatcaacggcaagctgaacagactgatcggcaagaccaacgagaagttccatcagatcgagaaggagttcagcgaggtggagggcagaatccaagacctggagaagtacgtggaggacaccaagatcgacctgtggagctacaacgccgagctgctggtggccctggagaatcagcacaccatcgacctgaccgacagcgagatgaacaagctgttcgagaagaccaagaagcagctgagagagaacgccgaggacatgggcaacggctgcttcaagatctaccacaagtgcgacaacgcctgcatcggcagcatcagaaacggcacctacgaccacgacgtgtacagagacgaggccctgaacaacagatttcagatcaagggcgtgtga

SEQ ID NO: 13 - DNA, 1503 bp, synthetic construct
  source 1..1503: mol_type = other DNA
atgcctatgggcagcctgcagcccctggccaccctgtacctgctgggcatgctggtggccagcgtgctggccggcggcagcggcggcggcggcagcggatccaacatcaccaacctctgccccttcgacgaggtgttcaacgccaccagattcgccagcgtctacgcctggaaccggaagcggatcagcaattgcgtggccgactacagcgtgctgtacaacctcgcccctttcttcaccttcaagtgctacggcgtgtcccctaccaagctgaacgatctgtgcttcaccaacgtgtacgccgacagcttcgtgatcaggggcgacgaagtgcgacagatcgccccaggacagacaggaaacatcgccgattacaactacaagctgcccgacgacttcaccggttgcgtgatcgcttggaacagcaacaagctggacagcaaggtctccggcaactacaactaccggtaccggctgttccggaagtccaacctgaagcccttcgagcgggacatcagcacagagatctaccaggccggcaacaaaccttgtaacggcgtggcgggcgtcaactgctacttccctctgcggagctacagcttccggcctacatacggcgtgggacaccagccttacagggtggtggtgctgagcttcgaactgctgcacgctccagccaccgtctgcggacctaaaaagagcaccaacctggtgaagaacaagtgcgtgaatttcggcggcagcggcgccatcggcggctacatccccgaggcccccagagacggccaggcctacgtgagaaaggacggcgagtgggtgctgctgagcaccttcctgggcggcagcctgggcggcggcggcagcggcagcaacatcaccaacctgtgccccttcggcgaggtgttcaacgccaccagattcgccagcgtgtacgcctggaacagaaagagaatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcagaggcgacgaggtgagacagatcgcccccggccagaccggcaagatcgccgactacaactacaagctgcccgacgacttcaccggctgcgtgatcgcctggaacagcaacaacctggacagcaaggtgggcggcaactacaactacctgtacagactgttcagaaagagcaacctgaagcccttcgagagagacatcagcaccgagatctaccaggccggcagcaccccctgcaacggcgtggagggcttcaactgctacttccccctgcagagctacggcttccagcccaccaacggcgtgggctaccagccctacagagtggtggtgctgagcttcgagctgctgcacgcccccgccaccgtgtgcggccccaagaagtccaccaacctggtgaagaacaagtgcgtgaacttc

SEQ ID NO: 14 - DNA, 52 bp, synthetic construct
  source 1..52: mol_type = other DNA
ggataggcggcgcatgagagaagcccagaccaattacctacccaaagccacc

SEQ ID NO: 15 - DNA, 118 bp, synthetic construct
  source 1..118: mol_type = other DNA
cggccgcatacagcagcaattggcaagctgcttacatagaactcgcggcgattggcatgccgccttaaaatttttcttttcttttccgaatcggattttgtttttaatatttaagctt

SEQ ID NO: 16 - RNA, 125 nt, synthetic construct
  source 1..125: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 17 - RNA, 89 nt, synthetic construct
  source 1..89: mol_type = other RNA
gccatcggcggctacatccccgaggcccccagagacggccaggcctacgtgagaaaggacggcgagtgggtgctgctgagcaccttcct

SEQ ID NO: 18 - RNA, 72 nt, synthetic construct
  source 1..72: mol_type = other RNA
atgcctatgggcagcctgcagcccctggccaccctgtacctgctgggcatgctggtggccagcgtgctggcc

SEQ ID NO: 19 - RNA, 633 nt, synthetic construct
  source 1..633: mol_type = other RNA
aacatcaccaacctctgccccttcgacgaggtgttcaacgccaccagattcgccagcgtctacgcctggaaccggaagcggatcagcaattgcgtggccgactacagcgtgctgtacaacctcgcccctttcttcaccttcaagtgctacggcgtgtcccctaccaagctgaacgatctgtgcttcaccaacgtgtacgccgacagcttcgtgatcaggggcgacgaagtgcgacagatcgccccaggacagacaggaaacatcgccgattacaactacaagctgcccgacgacttcaccggttgcgtgatcgcttggaacagcaacaagctggacagcaaggtctccggcaactacaactaccggtaccggctgttccggaagtccaacctgaagcccttcgagcgggacatcagcacagagatctaccaggccggcaacaaaccttgtaacggcgtggcgggcgtcaactgctacttccctctgcggagctacagcttccggcctacatacggcgtgggacaccagccttacagggtggtggtgctgagcttcgaactgctgcacgctccagccaccgtctgcggacctaaaaagagcaccaacctggtgaagaacaagtgcgtgaatttc

SEQ ID NO: 20 - RNA, 633 nt, synthetic construct
  source 1..633: mol_type = other RNA
aacatcaccaacctgtgccccttcggcgaggtgttcaacgccaccagattcgccagcgtgtacgcctggaacagaaagagaatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcagaggcgacgaggtgagacagatcgcccccggccagaccggcaagatcgccgactacaactacaagctgcccgacgacttcaccggctgcgtgatcgcctggaacagcaacaacctggacagcaaggtgggcggcaactacaactacctgtacagactgttcagaaagagcaacctgaagcccttcgagagagacatcagcaccgagatctaccaggccggcagcaccccctgcaacggcgtggagggcttcaactgctacttccccctgcagagctacggcttccagcccaccaacggcgtgggctaccagccctacagagtggtggtgctgagcttcgagctgctgcacgcccccgccaccgtgtgcggccccaagaagtccaccaacctggtgaagaacaagtgcgtgaacttc

SEQ ID NO: 21 - RNA, 33 nt, synthetic construct
  source 1..33: mol_type = other RNA
ggcggcagcctgggcggcggcggcagcggcagc

SEQ ID NO: 22 - RNA, 1587 nt, synthetic construct
  source 1..1587: mol_type = other RNA
atgaaggccaacctgctggtcctgctgtgcgccctggccgctgccgacgccgacaccatctgcatcggctaccacgccaacaacagcaccgacaccgtggacaccgtgctggagaagaacgtgaccgtgacccacagcgtgaacctgctggaggacagccacaacggcaagctgtgcagactgaagggcatcgcccccctgcagctgggcaagtgcaacatcgccggctggctgctgggcaaccccgagtgcgaccccctgctgcccgtgagaagctggagctacatcgtggagacccccaacagcgagaacggcatctgctaccccggcgacttcatcgactacgaggagctgagagagcagctgagcagcgtgagcagcttcgagagattcgagatcttccccaaggagagcagctggcccaaccacaacaccaacggcgtgaccgccgcctgcagccacgagggcaagagcagcttctacagaaacctgctgtggctgaccgagaaggagggcagctaccccaagctgaagaactcctatgtgaacaagaagggcaaagaggtgctggtgctgtggggcatccaccacccccccaacagcaaggagcagcagaacctgtatcagaacgagaacgcctacgtgagcgtggtgacaagcaactacaacagaagattcacccccgagatcgccgagagacccaaggtgagagaccaagccggcagaatgaactactactggaccctgctgaagcccggcgacaccatcatcttcgaggccaacggcaacctgatcgcccctatgtacgccttcgccctgagcagaggcttcggcagcggcatcatcacaagcaacgctagcatgcacgagtgcaacaccaagtgtcagacccccctgggcgccatcaacagcagcctgccctatcagaacatccaccccgtgaccatcggcgagtgccccaagtacgtgagaagcgccaagctgagaatggtgaccggcctgagaaacatccctagcattcagagcagaggcctgttcggcgccatcgccggcttcatcgagggcggctggaccggcatgatcgacggctggtacggctaccaccatcagaacgagcaaggcagcggctacgccgccgatcaaaagagcacacagaacgccatcaacggcatcaccaacaaggtgaacaccgtgatcgagaagatgaacattcagttcaccgccgtgggcaaggagttcaacaagctggagaagagaatggagaacctcaacaagaaggtggacgacggcttcctggacatctggacctacaacgccgagctgctggtgctgctggagaacgagagaaccctggacttccacgacagcaacgtgaagaacctgtacgagaaggtcaagagccaactgaagaacaatgccaaggagatcggcaacggctgcttcgagttctaccacaagtgcgacaacgagtgcatggagagcgtgagaaacggcacctacgactaccccaagtacagcgaggagagcaagctgaacagagagaaggtcgacggcgtgaagctggagagcatgggcatctatcagatc

SEQ ID NO: 23 - RNA, 1563 nt, synthetic construct
  source 1..1563: mol_type = other RNA
aagacaatcatcgccctgagccacatcctgtgcctggtgttcgctcagaagctgcccggcaacgacaacagcaccgccaccctgtgcctgggccaccacgccgtgcccaacggcaccatcgtgaaaacaatcaccaacgatcagatcgaggtgaccaacgccaccgagctggtgcagaacagcagcatcggcgagatctgcgacagcccccatcagatcctggacggcgagaactgcaccctgatcgacgctctgctgggcgatcctcagtgcgacggctttcagaacaagaagtgggacctgttcgtggagagaagcaaggcctacagcaactgctacccctacgatgtccccgactacgctagcctgagaagcctggtggctagcagcggcaccctggagttcaacaacgagagcttcaactggaccggcgtgacacagaacggcacaagcagcgcctgcatcagaagaagcaacaacagcttcttcagcagactgaactggctgacacagctgaacttcaagtaccccgccctgaacgtgaccatgcccaacaacgagcagttcgacaagctgtacatctggggcgtgcaccaccccgtgaccgacaaggatcagatcttcctgtacgctcagagcagcggcagaatcaccgtgagcaccaagagaagccaacaagccgtgatccccaacatcggctacagacctagaatcagaaacatccctagcagaatcagcatctactggaccatcgtgaagcccggcgacatcctgctgatcaacagcaccggcaacctgatcgcccctagaggctacttcaagatcagaagcggcaagagcagcatcatgagaagcgacgcccccatcggcaagtgcaacagcgagtgcatcacccccaacggcagcatccccaatgataagccctttcagaacgtgaacagaatcacctacggcgcctgccctagatacgtgaagcagagcaccctgaagctggccaccggcatgagaaacgtgcccgagaagcagacaagaggcatcttcggcgccatcgccggcttcatcgagaacggctgggagggcatggtggacggctggtacggcttcagacatcagaacagcgagggcagaggccaagccgccgacctcaagagcacccaagccgccatcgatcagatcaacggcaagctgaacagactgatcggcaagaccaacgagaagttccatcagatcgagaaggagttcagcgaggtggagggcagaatccaagacctggagaagtacgtggaggacaccaagatcgacctgtggagctacaacgccgagctgctggtggccctggagaatcagcacaccatcgacctgaccgacagcgagatgaacaagctgttcgagaagaccaagaagcagctgagagagaacgccgaggacatgggcaacggctgcttcaagatctaccacaagtgcgacaacgcctgcatcggcagcatcagaaacggcacctacgaccacgacgtgtacagagacgaggccctgaacaacagatttcagatcaagggcgtgtga

SEQ ID NO: 24 - RNA, 52 nt, synthetic construct
  source 1..52: mol_type = other RNA
ggataggcggcgcatgagagaagcccagaccaattacctacccaaagccacc

SEQ ID NO: 25 - RNA, 118 nt, synthetic construct
  source 1..118: mol_type = other RNA
cggccgcatacagcagcaattggcaagctgcttacatagaactcgcggcgattggcatgccgccttaaaatttttcttttcttttccgaatcggattttgtttttaatatttaagctt

SEQ ID NO: 26 - RNA, 1804 nt, synthetic construct
  source 1..1804: mol_type = other RNA
ggataggcggcgcatgagagaagcccagaccaattacctacccaaagccaccatgcctatgggcagcctgcagcccctggccaccctgtacctgctgggcatgctggtggccagcgtgctggccggcggcagcggcggcggcggcagcggatccaacatcaccaacctctgccccttcgacgaggtgttcaacgccaccagattcgccagcgtctacgcctggaaccggaagcggatcagcaattgcgtggccgactacagcgtgctgtacaacctcgcccctttcttcaccttcaagtgctacggcgtgtcccctaccaagctgaacgatctgtgcttcaccaacgtgtacgccgacagcttcgtgatcaggggcgacgaagtgcgacagatcgccccaggacagacaggaaacatcgccgattacaactacaagctgcccgacgacttcaccggttgcgtgatcgcttggaacagcaacaagctggacagcaaggtctccggcaactacaactaccggtaccggctgttccggaagtccaacctgaagcccttcgagcgggacatcagcacagagatctaccaggccggcaacaaaccttgtaacggcgtggcgggcgtcaactgctacttccctctgcggagctacagcttccggcctacatacggcgtgggacaccagccttacagggtggtggtgctgagcttcgaactgctgcacgctccagccaccgtctgcggacctaaaaagagcaccaacctggtgaagaacaagtgcgtgaatttcggcggcagcggcgccatcggcggctacatccccgaggcccccagagacggccaggcctacgtgagaaaggacggcgagtgggtgctgctgagcaccttcctgggcggcagcctgggcggcggcggcagcggcagcaacatcaccaacctgtgccccttcggcgaggtgttcaacgccaccagattcgccagcgtgtacgcctggaacagaaagagaatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcagaggcgacgaggtgagacagatcgcccccggccagaccggcaagatcgccgactacaactacaagctgcccgacgacttcaccggctgcgtgatcgcctggaacagcaacaacctggacagcaaggtgggcggcaactacaactacctgtacagactgttcagaaagagcaacctgaagcccttcgagagagacatcagcaccgagatctaccaggccggcagcaccccctgcaacggcgtggagggcttcaactgctacttccccctgcagagctacggcttccagcccaccaacggcgtgggctaccagccctacagagtggtggtgctgagcttcgagctgctgcacgcccccgccaccgtgtgcggccccaagaagtccaccaacctggtgaagaacaagtgcgtgaacttctgatgacggccgcatacagcagcaattggcaagctgcttacatagaactcgcggcgattggcatgccgccttaaaatttttcttttcttttccgaatcggattttgtttttaatatttaagcttaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 27 - RNA, 1825 nt, synthetic construct
  source 1..1825: mol_type = other RNA
ggataggcggcgcatgagagaagcccagaccaattacctacccaaagccaccatgcctatgggcagcctgcagcccctggccaccctgtacctgctgggcatgctggtggccagcgtgctggccggcggcagcggcggcggcggcagcggatccaacatcaccaacctctgccccttcgacgaggtgttcaacgccaccagattcgccagcgtctacgcctggaaccggaagcggatcagcaattgcgtggccgactacagcgtgctgtacaacctcgcccctttcttcaccttcaagtgctacggcgtgtcccctaccaagctgaacgatctgtgcttcaccaacgtgtacgccgacagcttcgtgatcaggggcgacgaagtgcgacagatcgccccaggacagacaggaaacatcgccgattacaactacaagctgcccgacgacttcaccggttgcgtgatcgcttggaacagcaacaagctggacagcaaggtctccggcaactacaactaccggtaccggctgttccggaagtccaacctgaagcccttcgagcgggacatcagcacagagatctaccaggccggcaacaaaccttgtaacggcgtggcgggcgtcaactgctacttccctctgcggagctacagcttccggcctacatacggcgtgggacaccagccttacagggtggtggtgctgagcttcgaactgctgcacgctccagccaccgtctgcggacctaaaaagagcaccaacctggtgaagaacaagtgcgtgaatttcggcggcagcctgggcggcggcggcagcggcagcgccatcggcggctacatccccgaggcccccagagacggccaggcctacgtgagaaaggacggcgagtgggtgctgctgagcaccttcctgggcggcagcctgggcggcggcggcagcggcagcaacatcaccaacctgtgccccttcggcgaggtgttcaacgccaccagattcgccagcgtgtacgcctggaacagaaagagaatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcagaggcgacgaggtgagacagatcgcccccggccagaccggcaagatcgccgactacaactacaagctgcccgacgacttcaccggctgcgtgatcgcctggaacagcaacaacctggacagcaaggtgggcggcaactacaactacctgtacagactgttcagaaagagcaacctgaagcccttcgagagagacatcagcaccgagatctaccaggccggcagcaccccctgcaacggcgtggagggcttcaactgctacttccccctgcagagctacggcttccagcccaccaacggcgtgggctaccagccctacagagtggtggtgctgagcttcgagctgctgcacgcccccgccaccgtgtgcggccccaagaagtccaccaacctggtgaagaacaagtgcgtgaacttctgatgacggccgcatacagcagcaattggcaagctgcttacatagaactcgcggcgattggcatgccgccttaaaatttttcttttcttttccgaatcggattttgtttttaatatttaagcttaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 28 - RNA, 1804 nt, synthetic construct
  source 1..1804: mol_type = other RNA
ggataggcggcgcatgagagaagcccagaccaattacctacccaaagccaccatgcctatgggcagcctgcagcccctggccaccctgtacctgctgggcatgctggtggccagcgtgctggccggcggcagcggcggcggcggcagcggatccaacatcaccaacctgtgccccttcggcgaggtgttcaacgccaccagattcgccagcgtgtacgcctggaacagaaagagaatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcagaggcgacgaggtgagacagatcgcccccggccagaccggcaagatcgccgactacaactacaagctgcccgacgacttcaccggctgcgtgatcgcctggaacagcaacaacctggacagcaaggtgggcggcaactacaactacctgtacagactgttcagaaagagcaacctgaagcccttcgagagagacatcagcaccgagatctaccaggccggcagcaccccctgcaacggcgtggagggcttcaactgctacttccccctgcagagctacggcttccagcccaccaacggcgtgggctaccagccctacagagtggtggtgctgagcttcgagctgctgcacgcccccgccaccgtgtgcggccccaagaagtccaccaacctggtgaagaacaagtgcgtgaacttcggcggcagcggcgccatcggcggctacatccccgaggcccccagagacggccaggcctacgtgagaaaggacggcgagtgggtgctgctgagcaccttcctgggcggcagcctgggcggcggcggcagcggcagcaacatcaccaacctctgccccttcgacgaggtgttcaacgccaccagattcgccagcgtctacgcctggaaccggaagcggatcagcaattgcgtggccgactacagcgtgctgtacaacctcgcccctttcttcaccttcaagtgctacggcgtgtcccctaccaagctgaacgatctgtgcttcaccaacgtgtacgccgacagcttcgtgatcaggggcgacgaagtgcgacagatcgccccaggacagacaggaaacatcgccgattacaactacaagctgcccgacgacttcaccggttgcgtgatcgcttggaacagcaacaagctggacagcaaggtctccggcaactacaactaccggtaccggctgttccggaagtccaacctgaagcccttcgagcgggacatcagcacagagatctaccaggccggcaacaaaccttgtaacggcgtggcgggcgtcaactgctacttccctctgcggagctacagcttccggcctacatacggcgtgggacaccagccttacagggtggtggtgctgagcttcgaactgctgcacgctccagccaccgtctgcggacctaaaaagagcaccaacctggtgaagaacaagtgcgtgaatttctgatgacggccgcatacagcagcaattggcaagctgcttacatagaactcgcggcgattggcatgccgccttaaaatttttcttttcttttccgaatcggattttgtttttaatatttaagcttaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 29 - RNA, 3586 nt, synthetic construct
  source 1..3586: mol_type = other RNA
ggataggcggcgcatgagagaagcccagaccaattacctacccaaagccaccatgaaggccaacctgctggtcctgctgtgcgccctggccgctgccgacgccgacaccatctgcatcggctaccacgccaacaacagcaccgacaccgtggacaccgtgctggagaagaacgtgaccgtgacccacagcgtgaacctgctggaggacagccacaacggcaagctgtgcagactgaagggcatcgcccccctgcagctgggcaagtgcaacatcgccggctggctgctgggcaaccccgagtgcgaccccctgctgcccgtgagaagctggagctacatcgtggagacccccaacagcgagaacggcatctgctaccccggcgacttcatcgactacgaggagctgagagagcagctgagcagcgtgagcagcttcgagagattcgagatcttccccaaggagagcagctggcccaaccacaacaccaacggcgtgaccgccgcctgcagccacgagggcaagagcagcttctacagaaacctgctgtggctgaccgagaaggagggcagctaccccaagctgaagaactcctatgtgaacaagaagggcaaagaggtgctggtgctgtggggcatccaccacccccccaacagcaaggagcagcagaacctgtatcagaacgagaacgcctacgtgagcgtggtgacaagcaactacaacagaagattcacccccgagatcgccgagagacccaaggtgagagaccaagccggcagaatgaactactactggaccctgctgaagcccggcgacaccatcatcttcgaggccaacggcaacctgatcgcccctatgtacgccttcgccctgagcagaggcttcggcagcggcatcatcacaagcaacgctagcatgcacgagtgcaacaccaagtgtcagacccccctgggcgccatcaacagcagcctgccctatcagaacatccaccccgtgaccatcggcgagtgccccaagtacgtgagaagcgccaagctgagaatggtgaccggcctgagaaacatccctagcattcagagcagaggcctgttcggcgccatcgccggcttcatcgagggcggctggaccggcatgatcgacggctggtacggctaccaccatcagaacgagcaaggcagcggctacgccgccgatcaaaagagcacacagaacgccatcaacggcatcaccaacaaggtgaacaccgtgatcgagaagatgaacattcagttcaccgccgtgggcaaggagttcaacaagctggagaagagaatggagaacctcaacaagaaggtggacgacggcttcctggacatctggacctacaacgccgagctgctggtgctgctggagaacgagagaaccctggacttccacgacagcaacgtgaagaacctgtacgagaaggtcaagagccaactgaagaacaatgccaaggagatcggcaacggctgcttcgagttctaccacaagtgcgacaacgagtgcatggagagcgtgagaaacggcacctacgactaccccaagtacagcgaggagagcaagctgaacagagagaaggtcgacggcgtgaagctggagagcatgggcatctatcagatcggcggcagcggcgccatcggcggctacatccccgaggcccccagagacggccaggcctacgtgagaaaggacggcgagtgggtgctgctgagcaccttcctgggcggcagcctgggcggcggcggcagcggcagcaagacaatcatcgccctgagccacatcctgtgcctggtgttcgctcagaagctgcccggcaacgacaacagcaccgccaccctgtgcctgggccaccacgccgtgcccaacggcaccatcgtgaaaacaatcaccaacgatcagatcgaggtgaccaacgccaccgagctggtgcagaacagcagcatcggcgagatctgcgacagcccccatcagatcctggacggcgagaactgcaccctgatcgacgctctgctgggcgatcctcagtgcgacggctttcagaacaagaagtgggacctgttcgtggagagaagcaaggcctacagcaactgctacccctacgatgtccccgactacgctagcctgagaagcctggtggctagcagcggcaccctggagttcaacaacgagagcttcaactggaccggcgtgacacagaacggcacaagcagcgcctgcatcagaagaagcaacaacagcttcttcagcagactgaactggctgacacagctgaacttcaagtaccccgccctgaacgtgaccatgcccaacaacgagcagttcgacaagctgtacatctggggcgtgcaccaccccgtgaccgacaaggatcagatcttcctgtacgctcagagcagcggcagaatcaccgtgagcaccaagagaagccaacaagccgtgatccccaacatcggctacagacctagaatcagaaacatccctagcagaatcagcatctactggaccatcgtgaagcccggcgacatcctgctgatcaacagcaccggcaacctgatcgcccctagaggctacttcaagatcagaagcggcaagagcagcatcatgagaagcgacgcccccatcggcaagtgcaacagcgagtgcatcacccccaacggcagcatccccaatgataagccctttcagaacgtgaacagaatcacctacggcgcctgccctagatacgtgaagcagagcaccctgaagctggccaccggcatgagaaacgtgcccgagaagcagacaagaggcatcttcggcgccatcgccggcttcatcgagaacggctgggagggcatggtggacggctggtacggcttcagacatcagaacagcgagggcagaggccaagccgccgacctcaagagcacccaagccgccatcgatcagatcaacggcaagctgaacagactgatcggcaagaccaacgagaagttccatcagatcgagaaggagttcagcgaggtggagggcagaatccaagacctggagaagtacgtggaggacaccaagatcgacctgtggagctacaacgccgagctgctggtggccctggagaatcagcacaccatcgacctgaccgacagcgagatgaacaagctgttcgagaagaccaagaagcagctgagagagaacgccgaggacatgggcaacggctgcttcaagatctaccacaagtgcgacaacgcctgcatcggcagcatcagaaacggcacctacgaccacgacgtgtacagagacgaggccctgaacaacagatttcagatcaagggcgtgtgatgatgacggccgcatacagcagcaattggcaagctgcttacatagaactcgcggcgattggcatgccgccttaaaatttttcttttcttttccgaatcggattttgtttttaatatttaagcttaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 30 - RNA, 1888 nt, synthetic construct
  source 1..1888: mol_type = other RNA
ggataggcggcgcatgagagaagcccagaccaattacctacccaaagccaccatgaaggccaacctgctggtcctgctgtgcgccctggccgctgccgacgccgacaccatctgcatcggctaccacgccaacaacagcaccgacaccgtggacaccgtgctggagaagaacgtgaccgtgacccacagcgtgaacctgctggaggacagccacaacggcaagctgtgcagactgaagggcatcgcccccctgcagctgggcaagtgcaacatcgccggctggctgctgggcaaccccgagtgcgaccccctgctgcccgtgagaagctggagctacatcgtggagacccccaacagcgagaacggcatctgctaccccggcgacttcatcgactacgaggagctgagagagcagctgagcagcgtgagcagcttcgagagattcgagatcttccccaaggagagcagctggcccaaccacaacaccaacggcgtgaccgccgcctgcagccacgagggcaagagcagcttctacagaaacctgctgtggctgaccgagaaggagggcagctaccccaagctgaagaactcctatgtgaacaagaagggcaaagaggtgctggtgctgtggggcatccaccacccccccaacagcaaggagcagcagaacctgtatcagaacgagaacgcctacgtgagcgtggtgacaagcaactacaacagaagattcacccccgagatcgccgagagacccaaggtgagagaccaagccggcagaatgaactactactggaccctgctgaagcccggcgacaccatcatcttcgaggccaacggcaacctgatcgcccctatgtacgccttcgccctgagcagaggcttcggcagcggcatcatcacaagcaacgctagcatgcacgagtgcaacaccaagtgtcagacccccctgggcgccatcaacagcagcctgccctatcagaacatccaccccgtgaccatcggcgagtgccccaagtacgtgagaagcgccaagctgagaatggtgaccggcctgagaaacatccctagcattcagagcagaggcctgttcggcgccatcgccggcttcatcgagggcggctggaccggcatgatcgacggctggtacggctaccaccatcagaacgagcaaggcagcggctacgccgccgatcaaaagagcacacagaacgccatcaacggcatcaccaacaaggtgaacaccgtgatcgagaagatgaacattcagttcaccgccgtgggcaaggagttcaacaagctggagaagagaatggagaacctcaacaagaaggtggacgacggcttcctggacatctggacctacaacgccgagctgctggtgctgctggagaacgagagaaccctggacttccacgacagcaacgtgaagaacctgtacgagaaggtcaagagccaactgaagaacaatgccaaggagatcggcaacggctgcttcgagttctaccacaagtgcgacaacgagtgcatggagagcgtgagaaacggcacctacgactaccccaagtacagcgaggagagcaagctgaacagagagaaggtcgacggcgtgaagctggagagcatgggcatctatcagatctgatgacggccgcatacagcagcaattggcaagctgcttacatagaactcgcggcgattggcatgccgccttaaaatttttcttttcttttccgaatcggattttgtttttaatatttaagcttaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 31 - RNA, 1864 nt, synthetic construct
  source 1..1864: mol_type = other RNA
ggataggcggcgcatgagagaagcccagaccaattacctacccaaagccaccatgaagacaatcatcgccctgagccacatcctgtgcctggtgttcgctcagaagctgcccggcaacgacaacagcaccgccaccctgtgcctgggccaccacgccgtgcccaacggcaccatcgtgaaaacaatcaccaacgatcagatcgaggtgaccaacgccaccgagctggtgcagaacagcagcatcggcgagatctgcgacagcccccatcagatcctggacggcgagaactgcaccctgatcgacgctctgctgggcgatcctcagtgcgacggctttcagaacaagaagtgggacctgttcgtggagagaagcaaggcctacagcaactgctacccctacgatgtccccgactacgctagcctgagaagcctggtggctagcagcggcaccctggagttcaacaacgagagcttcaactggaccggcgtgacacagaacggcacaagcagcgcctgcatcagaagaagcaacaacagcttcttcagcagactgaactggctgacacagctgaacttcaagtaccccgccctgaacgtgaccatgcccaacaacgagcagttcgacaagctgtacatctggggcgtgcaccaccccgtgaccgacaaggatcagatcttcctgtacgctcagagcagcggcagaatcaccgtgagcaccaagagaagccaacaagccgtgatccccaacatcggctacagacctagaatcagaaacatccctagcagaatcagcatctactggaccatcgtgaagcccggcgacatcctgctgatcaacagcaccggcaacctgatcgcccctagaggctacttcaagatcagaagcggcaagagcagcatcatgagaagcgacgcccccatcggcaagtgcaacagcgagtgcatcacccccaacggcagcatccccaatgataagccctttcagaacgtgaacagaatcacctacggcgcctgccctagatacgtgaagcagagcaccctgaagctggccaccggcatgagaaacgtgcccgagaagcagacaagaggcatcttcggcgccatcgccggcttcatcgagaacggctgggagggcatggtggacggctggtacggcttcagacatcagaacagcgagggcagaggccaagccgccgacctcaagagcacccaagccgccatcgatcagatcaacggcaagctgaacagactgatcggcaagaccaacgagaagttccatcagatcgagaaggagttcagcgaggtggagggcagaatccaagacctggagaagtacgtggaggacaccaagatcgacctgtggagctacaacgccgagctgctggtggccctggagaatcagcacaccatcgacctgaccgacagcgagatgaacaagctgttcgagaagaccaagaagcagctgagagagaacgccgaggacatgggcaacggctgcttcaagatctaccacaagtgcgacaacgcctgcatcggcagcatcagaaacggcacctacgaccacgacgtgtacagagacgaggccctgaacaacagatttcagatcaagggcgtgtgatgacggccgcatacagcagcaattggcaagctgcttacatagaactcgcggcgattggcatgccgccttaaaatttttcttttcttttccgaatcggattttgtttttaatatttaagcttaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 32 - protein, 100 aa, synthetic construct
  VARIANT 11..100: note = GGGGS repeats may be deleted
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 33 - protein, 20 aa, synthetic construct
  VARIANT 3..20: note = Residues may be deleted
GGGGGGGGGGGGGGGGGGGG

SEQ ID NO: 34 - protein, 36 aa, synthetic construct
  VARIANT 13..36: note = GGSGGD repeats may be deleted
GGSGGDGGSGGDGGSGGDGGSGGDGGSGGDGGSGGD

SEQ ID NO: 35 - protein, 36 aa, synthetic construct
  VARIANT 13..36: note = GGSGGE repeats may be deleted
GGSGGEGGSGGEGGSGGEGGSGGEGGSGGEGGSGGE

SEQ ID NO: 36 - protein, 33 aa, synthetic construct
  VARIANT 12..33: note = GGGSGSGGGGS repeats may be deleted
GGGSGSGGGGSGGGSGSGGGGSGGGSGSGGGGS

SEQ ID NO: 37 - protein, 33 aa, synthetic construct
  VARIANT 12..33: note = GGGGGPGGGGP repeats may be deleted
GGGGGPGGGGPGGGGGPGGGGPGGGGGPGGGGP

SEQ ID NO: 38 - protein, 4 aa, synthetic construct
GGSG

SEQ ID NO: 39 - protein, 11 aa, synthetic construct
GGSLGGGGSGS

SEQ ID NO: 40 - RNA, 300 nt, unidentified
  source 1..300: mol_type = unassigned RNA; note = PolyA tail
  variation 11..300: note = nucleotides may be deleted
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa